(19) United States
(12) Patent Application Publication
(10) Pub. No.: US 2026/0146255 A1
(43) Pub. Date: May 28, 2026

(54) MODULATION OF NADPH GENERATION BY RECOMBINANT YEAST HOST CELL DURING FERMENTATION

(71) Applicant: DANSTAR FERMENT AG, Zug (CH)

(72) Inventors: Ryan Skinner, Bethel, VT (US); Aaron Argyros, Lebanon, NH (US); Adam Simard, Lebanon, NH (US); Trisha Barrett, Bradford, VT (US)

(21) Appl. No.: 19/407,675

(22) Filed: Dec. 3, 2025

Related U.S. Application Data
(63) Continuation of application No. 17/299,984.
(60) Provisional application No. 62/776,910, filed on Dec. 7, 2018.

(51) Int. Cl. C12N 15/52; C12N 9/02; C12N 9/04; C12N 9/06; C12N 9/24; C12N 9/26; C12N 9/88; C12P 7/10

(52) U.S. Cl. CPC C12N 15/52; C12N 9/0006; C12N 9/0008; C12N 9/0016; C12N 9/2402; C12N 9/2408; C12N 9/2414; C12N 9/88; C12P 7/10; C12Y 101/01001; C12Y 101/01014; C12Y 101/01021; C12Y 101/01042; C12Y 101/01044; C12Y 101/01049; C12Y 101/01202; C12Y 101/01255; C12Y 102/01003; C12Y 102/01009; C12Y 102/01012; C12Y 104/01002; C12Y 104/01013; C12Y 302/01001; C12Y 302/0102; C12Y 302/01028; C12Y 402/0103

(57) ABSTRACT

The present disclosure concerns recombinant yeast host cells having a first genetic modification for downregulating a first metabolic pathway that converts NADP⁺ to NADPH, as well as a second genetic modification for upregulating a second metabolic pathway that converts NADP⁺ to NADPH. The second genetic modification allows the expression of a glyceraldehyde-3-phosphate dehydrogenase lacking phosphorylating activity, which can, in some embodiments, be from enzyme commission 1.2.1.9 or 1.2.1.90. The second pathway is distinct from the first metabolic pathway. The present disclosure also concerns a process for making and improving the yield of a fermented product, such as ethanol, using the recombinant yeast host cell.

### CROSS-REFERENCE TO RELATED APPLICATIONS

This application is a continuation of U.S. patent application Ser. No. 17/299,984, filed Jun. 4, 2021, which is a U.S. national phase application of PCT/IB2019/060527, filed Dec. 6, 2019, which claims priority from U.S. provisional application Ser. No. 62/776,910 filed on Dec. 7, 2018, all are herewith incorporated their entirety.

### REFERENCE TO AN ELECTRONIC SEQUENCE LISTING

The contents of the electronic sequence listing (580127_425C1_SL.xml; Size: 161, 173 bytes; and Date of Creation: Dec. 2, 2025) is herein incorporated by reference in its entirety.

### TECHNOLOGICAL FIELD

The present disclosure relates to a recombinant yeast host cell having modulated pathways for NADPH utilization and generation.

### BACKGROUND

Saccharomyces cerevisiae is the primary biocatalyst used in the commercial production of fuel ethanol. This organism is proficient in fermenting glucose to ethanol, often to concentrations greater than 20% (v/v). To further improve upon this ethanol yield, utilization of formate production as an alternate to glycerol as an electron sink, results in reduced glycerol secretion, has been engineered into yeast (e.g., WO2012138942). This strategy successfully reduces the production of the fermentation by-product glycerol, and increases valuable ethanol production by the strain.

It would be desirable for a corn ethanol producer, to be provided with an alternative recombinant yeast host cell which could provide higher ethanol yields, or which might provide other benefits such as tolerance to process upsets, fermentation rate, or new and/or improved enzymatic activities, relative to current commercially available strains. This approach could provide a novel alternative metabolic pathway, which when expressed in yeast, results in a higher ethanol yield and a lower glycerol yield during corn mash fermentations.

### SUMMARY

The present disclosure provides recombinant yeast host cells which redirect NADP⁺ from a first metabolic pathway towards a second metabolic pathway so as to upregulate the second metabolic pathway. The present disclosure concerns a recombinant yeast host cell having: i) one or more of a first genetic modification for downregulating a first metabolic pathway; and ii) one or more of a second genetic modification for upregulating a second metabolic pathway. The first metabolic pathway and the second metabolic pathway allow the conversion of NADP⁺ to NADPH. The first metabolic pathway is distinct from the second metabolic pathway.

According to a first aspect, the present disclosure concerns a recombinant yeast host cell having: i) one or more of a first genetic modification for downregulating a first metabolic pathway; and ii) one or more of a second genetic modification for upregulating a second metabolic pathway, wherein the one or more second genetic modification allows the expression of a glyceraldehyde-3-phosphate dehydrogenase lacking phosphorylating activity, wherein the glyceraldehyde-3-phosphate dehydrogenase is of enzyme commission (EC) 1.2.1.9 or 1.2.1.90. The first metabolic pathway and the second metabolic pathway allow the conversion of NADP⁺ to NADPH. The first metabolic pathway is distinct from the second metabolic pathway. In an embodiment, the first genetic modification comprises inactivation of at least one first native gene. In yet another embodiment, the first metabolic pathway is the pentose phosphate pathway. In still a further embodiment, the at least one first native gene comprises a zwf1 gene encoding a polypeptide having glucose-6-phosphate dehydrogenase activity, an ortholog of the zwf1 gene or a paralog of the zwf1 gene. In a specific embodiment, the polypeptide having glucose-6-phosphate dehydrogenase activity has the amino acid sequence of SEQ ID NO: 3, is a variant of SEQ ID NO: 3 having glucose-6-phosphate dehydrogenase activity, or is a fragment of SEQ ID NO: 3 having glucose-6-phosphate dehydrogenase activity. In another embodiment, the at least one first native gene comprises a gnd1 gene encoding a polypeptide having 6-phosphogluconate dehydrogenase activity, an ortholog of the gnd1 gene or a paralog of the gnd1 gene. In a further embodiment, the polypeptide having 6-phosphogluconate dehydrogenase activity has the amino acid sequence of SEQ ID NO: 4, is a variant of SEQ ID NO: 4 having 6-phosphogluconate dehydrogenase activity, or is a fragment of SEQ ID NO: 4 having 6-phosphogluconate dehydrogenase activity. In yet another embodiment, the at least one first native gene comprises a gnd2 gene encoding a polypeptide having 6-phosphogluconate dehydrogenase activity, an ortholog of the gnd2 gene or a paralog of the gnd2 gene. In a specific embodiment, polypeptide having 6-phosphogluconate dehydrogenase activity has the amino acid sequence of SEQ ID NO: 5, is a variant of SEQ ID NO: 5 having 6-phosphogluconate dehydrogenase activity, or is a fragment of SEQ ID NO: 5 having 6-phosphogluconate dehydrogenase activity. In another embodiment, the at least one first native gene comprises an ald6 gene encoding a polypeptide having aldehyde dehydrogenase activity, an ortholog of the ald6 gene or a paralog of the ald6 gene. In a specific embodiment, the polypeptide having aldehyde dehydrogenase activity has the amino acid sequence of SEQ ID NO: 6, is a variant of SEQ ID NO: 6 having aldehyde dehydrogenase activity, or is a fragment of SEQ ID NO: 6 having aldehyde dehydrogenase activity. In still another embodiment, the at least one first native gene comprises a idp1 gene encoding a polypeptide having isocitrate dehydrogenase activity, an ortholog of the ipd1 gene or a paralog of the ipd1 gene. In a further embodiment, the polypeptide having isocitrate dehydrogenase activity has the amino acid sequence of SEQ ID NO: 7, is a variant of SEQ ID NO: 7 having isocitrate dehydrogenase activity, or is a fragment of SEQ ID NO: 7 having isocitrate dehydrogenase activity. In another embodiment, the at least one first native gene comprises a idp2 gene encoding a polypeptide having isocitrate dehydrogenaseactivity, an ortholog of the ipd2 gene or a paralog of the ipd2 gene. In a further embodiment, the polypeptide having isocitrate dehydrogenase activity has the amino acid sequence of SEQ ID NO: 8, is a variant of SEQ ID NO: 8 having isocitrate dehydrogenase activity, or is a fragment of SEQ ID NO: 8 having isocitrate dehydrogenase activity. In another embodiment, the at least one first native gene comprises a idp3 gene encoding a polypeptide having isocitrate dehydrogenaseactivity, an ortholog of the ipd3 gene or a paralog of the ipd3 gene. In a further embodiment, the polypeptide having isocitrate dehydrogenase activity has the amino acid sequence of SEQ ID NO: 9, is a variant of SEQ ID NO: 9 having isocitrate dehydrogenase activity, or is a fragment of SEQ ID NO: 9 having isocitrate dehydrogenase activity. In still another embodiment, the one or more second genetic modification comprises introduction of one or more second heterologous nucleic acid molecule encoding the glyceraldehyde-3-phosphate dehydrogenase. In an embodiment, the recombinant has the one or more second heterologous nucleic acid molecule in an open reading frame of the first native gene. In another embodiment, the at least one first native gene has a native promoter. In a further embodiment, the one or more second heterologous nucleic acid molecule is under the control of the native promoter of the at least one first native gene. In yet another embodiment, the one or more second heterologous nucleic acid molecule is under the control of an heterologous promoter. In some embodiments, the heterologous promoter comprises the promoter of the ADH1, GPD1, HXT3, QCR8, PGI1, PFK1, FBA1, TDH2, PGK1, GPM1, ENO2, CDCl9, ZWF1, HOR7 and/or TPI1 gene. In yet another embodiment, the glyceraldehyde-3-phosphate dehydrogenase is of EC 1.2.1.90. In a specific embodiment, the glyceraldehyde-3-phosphate dehydrogenase is GAPN which can be derived from Streptococcus sp. and, in yet another embodiment, from Streptococcus mutans. In some embodiment, GAPN has the amino acid sequence of SEQ ID NO: 2, 47, 49, 51, 53, 55, 57, 59, 61, 72, 74, 76, 78, 80, 82, 84 or 86, is a variant of SEQ ID NO: 2, 47, 49, 51, 53, 55, 57, 59, 61, 72, 74, 76, 78, 80, 82, 84 or 86 having glyceraldehyde-3-phosphate dehydrogenase activity, or is a fragment of SEQ ID NO: 2, 47, 49, 51, 53, 55, 57, 59, 61, 72, 74, 76, 78, 80, 82, 84 or 86 having glyceraldehyde-3-phosphate dehydrogenase activity. In another embodiment, the glyceraldehyde-3-phosphate dehydrogenase is of EC 1.2.1.9. In some embodiment, the at least one first native gene has a first promoter. In still another embodiment, the recombinant yeast host cell has iii) one or more of a third genetic modification for upregulating a third metabolic pathway, wherein the third metabolic pathways allows the conversion of NADH to NAD⁺. In an embodiment, the one or more of the third genetic modification comprises introducing one or more third heterologous nucleic acid molecule encoding one or more of third polypeptide. In still another embodiment, the third metabolic pathway allows the production of ethanol. In a further embodiment, the one or more third polypeptide comprises a polypeptide having bifunctional alcohol/aldehyde dehydrogenase activity (which can have, for example, the amino acid sequence of SEQ ID NO: 10, be a variant of SEQ ID NO: 10 having bifunctional alcohol/aldehyde dehydrogenase activity, or be a fragment of SEQ ID NO: 10 having bifunctional alcohol/aldehyde dehydrogenase activity). In another embodiment, the one or more third polypeptide comprises a polypeptide having glutamate dehydrogenase activity (which can have, for example, the amino acid sequence of SEQ ID NO: 11, be a variant of SEQ ID NO: 11 having glutamate dehydrogenase activity, or be a fragment of SEQ ID NO: 11 having glutamate dehydrogenase activity). In another embodiment, the one or more third polypeptide comprises a polypeptide having alcohol dehydrogenase activity (which can have, for example, the amino acid sequence of any one of SEQ ID NO: 12 to 18, be a variant of any one of SEQ ID NO: 12 to 18 having NADH-dependent alcohol dehydrogenase activity, or be a fragment of any one of SEQ ID NO: 12 to 18 having NADH-dependent alcohol dehydrogenase activity). In an embodiment, the third metabolic pathway allows the production of 1,3-propanediol. In this specific embodiment, the one or more third heterologous polypeptide comprises a polypeptide having 1,3-propanediol dehydrogenase activity, optionally in combination with a polypeptide having glycerol dehydratase activase activity and a polypeptide having glycerol dehydratase activity. For example, the polypeptide having glycerol dehydratase activase activity can have the amino acid sequence of SEQ ID NO: 30, be a variant of the amino acid sequence of SEQ ID NO: 30 having glycerol dehydratase activase activity, or be a fragment of the amino acid sequence of SEQ ID NO: 30 having glycerol dehydratase activase activity. In yet another example, the polypeptide having glycerol dehydratase activity can have the amino acid sequence of SEQ ID NO: 32, be a variant of the amino acid sequence of SEQ ID NO: 32 having glycerol dehydratase activity, or be a fragment of the amino acid sequence of SEQ ID NO: 32 having glycerol dehydratase activity. In still another example, the polypeptide having 1,3-propanediol dehydrogenase activity can have the amino acid sequence of SEQ ID NO: 34, be a variant of the amino acid sequence of SEQ ID NO: 34 having 1,3-propanediol dehydrogenase activity, or be a fragment of the amino acid sequence of SEQ ID NO: 34 having 1,3-propanediol dehydrogenase activity. In another embodiment, the recombinant yeast host cell further has iv) one or more of a fourth genetic modification for upregulating a fourth metabolic pathway, wherein the fourth metabolic pathway allows the conversion of NAPDH to NADP⁺. In an embodiment, the one or more fourth genetic modification comprises introducing one or more fourth heterologous nucleic acid molecule encoding one or more fourth polypeptide. In another embodiment, the one or more fourth polypeptide comprises a polypeptide having aldose reductase activity. In a further embodiment, the polypeptide having aldose reductase activity comprises a polypeptide having mannitol dehydrogenase activity (which can have, for example, the amino acid sequence of SEQ ID NO: 19, be a variant of SEQ ID NO: 19 having aldose reductase activity, or be a fragment of SEQ ID NO: 19 having aldose reductase activity). In a further embodiment, the polypeptide having aldose reductase activity comprises a polypeptide having sorbitol dehydrogenase activity (which can have, for example, the amino acid sequence of SEQ ID NO: 20 or 21, be a variant of SEQ ID NO: 20 or 21 having sorbitol dehydrogenase activity, or be a fragment of SEQ ID NO: 20 or 21 having sorbitol dehydrogenase activity). In a further embodiment, the one or more fourth polypeptide comprises a polypeptide having NADP⁺-dependent alcohol dehydrogenase activity (which can have, for example, the amino acid sequence of any one of SEQ ID NO: 17 or 18, be a variant of any one of SEQ ID NO: 17 or 18 having NADP⁺-dependent alcohol dehydrogenase activity, or be a fragment of any one of SEQ ID NO: 17 or 18 having NADP⁺-dependent alcohol dehydrogenase activity). In another embodiment, the recombinant yeast host cell further has v) a fifth genetic modification for expressing a fifth polypeptide for increasing saccharolytic activity. In an embodiment, the fifth polypeptide comprises an enzyme having alpha-amylase activity and/or an enzyme having glucoamylase activity. In an embodiment, the enzyme having glucoamylase activity has the amino acid sequence of SEQ ID NO: 28 or 40, is a variant of the amino acid sequence of SEQ ID NO: 28 or 40 having glucoamylase activity, or is a fragment of the amino acid sequence of SEQ ID NO: 28 or 40 having glucoamylase activity. In a further embodiment, the fifth heterologous polypeptide comprises an enzyme having trehalase activity. For example, the enzyme having trehalase activity can have the amino acid sequence of SEQ ID NO: 38, can be a variant or the amino acid sequence of SEQ ID NO: 38 having trehalase activity, or can be a fragment of the amino acid sequence of SEQ ID NO: 38 having trehalase activity. In still another embodiment, the recombinant yeast host cell further has vi) a sixth genetic modification for expressing a sixth heterologous polypeptide for reducing the production of glycerol or facilitating the transport of glycerol in the recombinant yeast host cell. In an embodiment, the sixth heterologous polypeptide comprises a STL1 polypeptide having glycerol proton symporter activity. For example, the STL1 polypeptide can have the amino acid sequence of SEQ ID NO: 26, be a variant of the amino acid sequence of SEQ ID NO: 26 having glycerol proton symporter activity, or be a fragment of the amino acid sequence of SEQ ID NO: 26 having glycerol proton symporter activity. In still another embodiment, the sixth heterologous polypeptide comprises a GLT1 polypeptide having NAD (+)-dependent glutamate synthase activity and a GLN1 polypeptide having glutamine synthetase activity. In an embodiment, the GLT1 polypeptide has the amino acid sequence of SEQ ID NO: 43, is a variant of the amino acid sequence of SEQ ID NO: 43 having NAD (+)-dependent glutamate synthase activity or is a fragment of SEQ ID NO: 43 having NAD (+)-dependent glutamate synthase activity. In still another embodiment, the GLN1 polypeptide has the amino acid sequence of SEQ ID NO: 45, is a variant of the amino acid sequence of SEQ ID NO: 45 having glutamine synthetase activity or is a fragment of the amino acid sequence of SEQ ID NO: 45 having glutamine synthetase activity. In an embodiment, the recombinant yeast host cell is from the genus Saccharomyces and, in some additional embodiments, from the species Saccharomyces cerevisiae.

According to a second aspect, the present disclosure provides a process for converting a biomass into a fermentation product, the process comprises contacting the biomass with the recombinant yeast host cell defined herein to allow the conversion of at least a part of the biomass into the fermentation product. In an embodiment, the biomass comprises corn. In another embodiment, the corn is provided as a mash. In yet another embodiment, the fermentation product is ethanol. In yet a further embodiment, the recombinant yeast host cell increases ethanol production compared to a corresponding native yeast host cell lacking the first genetic modification and the second genetic modification. In another embodiment, the recombinant yeast host cell further decreases glycerol production compared to a corresponding native yeast host cell lacking the first genetic modification and the second genetic modification.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows a pathway schematic detailing NADPH regeneration by GAPN in zwf1 knockout (zwf1Δ) yeast cells. GAPN uses cofactor NADP⁺ to convert glyceraldehyde-3-phosphate into 3-phosphoglycerate (large curved arrow). Native zwf1 also uses cofactor NADP⁺ and allows for conversion of glucose-6-phosphate into gluconate-6-phosphate.

FIG. 2 shows the resulting fermentation products of wildtype and recombinant Saccharomyces cerevisiae strains fermented in Verduyn's media. Results are shown as the ethanol titer (bars, right axis, g/L) and the glycerol concentration (•, left axis in g/L) for strains M2390, M18646, M7153 and M18913.

FIG. 3 shows pathway schematics detailing the conversion of glyceraldehyde-3-phosphate into 3-phosphoglycerate by GAPN (EC1.2.1.9) and the conversion of glyceraldehyde-3-phosphate into 3-phospho-D-glyceroyl-phosphate by GDP1 (EC1.2.1.13). In the reaction presented in this figure, GAPN is a non-phosphorylating glyceraldehyde-3-phosphate dehydrogenase (GAPDH) having estimated ΔᵣG′^{m} of −36.1±1.1 kJ/mol, and therefore being thermodynamically very favorable. GDP1 is a phosphorylating GAPDH having estimated ΔᵣG′^{m} of 25.9±1.0 kJ/mol, and therefore being thermodynamically very unfavorable.

FIG. 4 shows a comparison of the thermodynamics of various glyceraldehyde-3-phosphate dehydrogenases (EC 1.2.1.9, EC 1.2.1.13, and EC 1.2.1.12) and ZWF1 (EC 1.1.1.49).

FIGS. 5A and 5B show a comparison of (FIG. 5A) a native glycolysis pathway schematic which produces net two molecules of ATP per glucose molecule, and (FIG. 5B) glycolysis pathway schematic using GDP1 (EC 1.2.1.13) which also produces net two molecules of ATP per glucose molecule. Molecule names contain extra capitals to illustrate components.

FIGS. 6A and 6B show a comparison of (FIG. 6A) a native glycolysis pathway schematic which produces net two molecules of ATP per glucose molecule, and (FIG. 6B) glycolysis pathway schematic using GAPN (EC 1.2.1.9) which does not result in any net gain of ATP per glucose molecule. Molecule names contain extra capitals to illustrate components.

FIGS. 7A and 7B shows a comparison of (FIG. 7A) a native glycolysis pathway schematic which produces net two molecules of ATP per glucose molecule, and (FIG. 7B) glycerol production pathway schematic which consumes two molecules of ATP per glucose molecule. Molecule names contain extra capitals to illustrate components.

FIG. 8 provides a schematic representation of the pentose phosphate pathway.

FIG. 9 provides the resulting fermentation products of a corn mash fermentation performed under permissive conditions. Results are shown as ethanol (g/L, bars, left axis), glucose (g/L, ▴, right axis) and glycerol (g/L, •, right axis) in function of strain tested.

FIG. 10 provides the resulting fermentation products of a corn mash fermentation performed under permissive conditions. Results are shown as ethanol (g/L, bars, left axis), glucose (g/L, ▴, right axis) and glycerol (g/L, •, right axis) in function of strain tested.

FIG. 11 provides the resulting fermentation products of a corn mash fermentation performed under permissive conditions. Results are shown as ethanol (g/L, bars, left axis), glucose (g/L, ▴, right axis) and glycerol (g/L, •, right axis) in function of strain tested.

FIGS. 12A to 12C provide the resulting fermentation products of a corn mash fermentation performed under (FIG. 12A) permissive, (FIG. 12B) lactic acid or (FIG. 12C) high temperature conditions. Results are shown as ethanol (g/L, bars, left axis), glucose (g/L, ▴, right axis) and glycerol (g/L, •, right axis) in function of strain tested.

FIGS. 13A to 13C provide the concentration of (FIG. 13A) ethanol (g/L), (FIG. 13B) glycerol (g/L) and (FIG. 13C) glucose (g/L) of a corn mash fermentation after 18 h (white bars), 27 h (diagonal hatch bars), 48 h (grey bars) and 65 h (black bars).

FIGS. 14A to 14C provide the resulting (FIG. 14A) fermentation yield (g of ethanol/g of glucose), (FIG. 14B) yeast-produced glycerol (g/L) and (FIG. 14C) dry cell weight of a culture of various yeast strains in Verduyn medium.

FIGS. 15A to 15D provide the resulting (FIGS. 15A and 15C) fermentation yield (g of ethanol/g of glucose) and (FIGS. 15B and 15D) yeast-produced glycerol (g/L) of a culture of various yeast strains in Verduyn medium.

### DETAILED DESCRIPTION

The present disclosure provides an alternative for reducing glycerol by diverting more carbon flux towards pyruvate by introducing a heterologous glyceraldehyde-3-phosphate dehydrogenase gene into the recombinant yeast host cell. This NADP⁺-dependent enzyme results in glycerol reduction and ethanol yield increases when engineered into yeast (Zhang et al., 2013). However, the full potential of this pathway is not realized if NADP⁺ and/or NAD⁺ cofactor availability is insufficient. To avoid this, the present disclosure provides for modification of a yeast host genome, including the inactivation of at least genes encoding for enzymes responsible for the production of NADPH. By inactivating NADPH generating enzymes and expressing heterologous NADP⁺-dependent glyceraldehyde-3-phosphate dehydrogenase, it is possible to create increased glycolytic flux resulting in reduced glycerol formation and increased ethanol titers during yeast fermentation.

The present disclosure thus provides a recombinant yeast host cell which downregulates a first metabolic pathway (which, in its native unaltered form allows the conversion of NADP⁺ to NADPH), and upregulates a second metabolic pathway that also allows the conversion of NADP⁺ to NADPH by expressing glyceraldehyde-3-phosphate dehydrogenase which converts NADP⁺ to NADPH, so as to increase the fermentation yield. In an embodiment, when a biomass (for example comprising corn) is fermented by the recombinant yeast host cell of the present disclosure, at the conclusion of a fermentation, the fermentation medium has less than 10 g/L, 9 g/L, 8 g/L, 7 g/L, 6 g/L, 5 g/L, 4 g/L, 3 g/L, 2 g/L or 1 g/L of glycerol. Alternatively or in combination, when a biomass (for example comprising corn) is fermented by the recombinant yeast host cell of the present disclosure, at the conclusion of a fermentation, the fermentation medium has less than 120 g/L, 110 g/L, 100 g/L, 90 g/L, 80 g/L, 70 g/L, 60 g/L, 50 g/L, 40 g/L, 30 g/L, 20 g/L or 10 g/L of glucose. Alternatively or in combination, when a biomass (for example comprising corn) is fermented by the recombinant yeast host cell of the present disclosure, at the conclusion of a permissive fermentation, the fermentation medium has at least 100 g/L, 105 g/L, 110 g/L, 115 g/L, 120 g/L, 125 g/L, 130 g/L, 135 g/L or 140 g/L of ethanol. Alternatively or in combination, when a biomass (for example comprising corn) is fermented by the recombinant yeast host cell of the present disclosure, at the conclusion of a stress fermentation, the fermentation medium has at least 50 g/L, 55 g/L, 60 g/L, 65 g/L, 70 g/L, 75 g/L, 80 g/L, 85 g/L or 90 g/L of ethanol.

### Recombinant Yeast Host Cell

The present disclosure concerns recombinant yeast host cells obtained by introducing at least two genetic modifications in a corresponding native yeast host cell. The genetic modification(s) in the recombinant yeast host cell of the present disclosure comprise one or more of a first genetic modification for downregulating a first pathway for conversion of NADP⁺ to NADPH, and one or more of a second genetic modification for upregulating a second pathway for conversion of NADP⁺ to NADPH that is distinct from the first pathway. The second genetic modification allows the expression of a glyceraldehyde-3-phosphate dehydrogenase lacking phosphorylating activity as described herein for conversion of NADP⁺ to NADPH.

In one embodiment, the glyceraldehyde-3-phosphate dehydrogenase does not have phosphorylating activity and can be of EC 1.2.1.90 or 1.2.1.9. Glyceraldehyde-3-phosphate dehydrogenases from EC 1.2.1.9 are also known as triosephosphate dehydrogenases catalyze the following reaction:

D-glyceraldehyde 3-phosphate+NADP⁺+H₂O <=>3-phospho-D-glycerate+NADPH Glyceraldehyde-3-phosphate dehydrogenase from EC 1.2.1.90 are also known as non-phosphorylating glyceraldehyde-3-phosphate dehydrogenase and catalyze the following reaction:

D-glyceraldehyde 3-phosphate+NAD(P)⁺+H₂O<=>3-phospho-D-glycerate+NAD(P)H

In some embodiments, the genetic modification(s) in the recombinant yeast host cell of the present disclosure comprise or consist essentially of or consist of a first genetic modification for downregulating a first pathway for conversion of NADP⁺ to NADPH, and one or more of a second genetic modification for upregulating a second pathway for conversion of NADP⁺ to NADPH that is distinct from the first pathway. The second genetic modification allows the expression of a glyceraldehyde-3-phosphate dehydrogenase lacking phosphorylating activity as described herein for conversion of NADP⁺ to NADPH. In one embodiment, the glyceraldehyde-3-phosphate dehydrogenase is of EC 1.2.1.9 or 1.2.1.90. In the context of the present disclosure, the expression “the genetic modification(s) in the recombinant yeast host consist essentially of a first genetic modification for downregulating a first pathway for conversion of NADP⁺ to NADPH, and one or more of a second genetic modification” refers to the fact that the recombinant yeast host cell only includes these genetic modifications to modulate NADPH levels but can nevertheless include other genetic modifications which are unrelated to the generation of NADPH.

In some embodiments, the genetic modifications in the recombinant yeast host cell further comprises one or more of a third genetic modification for upregulating a third metabolic pathway for the conversion of NADH to NAD⁺. In some alternative embodiments, the genetic modifications in the recombinant yeast host cell comprise or consist essentially of a first genetic modification, a second genetic modification and a third genetic modification.

In some embodiments, the genetic modifications in the recombinant yeast host cell further comprises one or more of a fourth genetic modification for upregulating a fourth metabolic pathway for the conversion of NADPH to NADP⁺. In some alternative embodiments, the genetic modifications in the recombinant yeast host cell comprise or consist essentially of a first genetic modification, a second genetic modification, and a fourth genetic modification (optionally in combination with a third genetic modification).

In some embodiments, the genetic modifications in the recombinant yeast host cell further comprises one or more of a fifth genetic modification for expressing a fifth polypeptide having saccharolytic activity. In some alternative embodiments, the genetic modifications in the recombinant yeast host cell comprise or consist essentially of a first genetic modification, a second genetic modification and a fifth genetic modification (optionally in combination with a third and/or fourth genetic modification).

In some embodiments, the genetic modifications in the recombinant yeast host cell further comprises one or more of a sixth genetic modification for expressing a sixth polypeptide for facilitating the transport of glycerol in the recombinant yeast host cell. In some alternative embodiments, the genetic modifications in the recombinant yeast host cell comprise or consist essentially of a first genetic modification, a second genetic modification and a sixth genetic modification (optionally in combination with a third, fourth and/or fifth genetic modification).

When the genetic modification is aimed at reducing or inhibiting the expression of a specific targeted gene (which is endogenous to the host cell), the genetic modifications can be made in one, two or all copies of the targeted gene(s). When the genetic modification is aimed at increasing the expression of a specific targeted gene, the genetic modification can be made in one or multiple genetic locations. In the context of the present disclosure, when recombinant yeast host cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from an heterologous cell or the recombinant yeast host cell itself. In the latter scenario, the nucleic acid residue(s) is (are) added at a genomic location which is different than the native genomic location. The genetic manipulations did not occur in nature and are the results of in vitro manipulations of the native yeast host cell.

When expressed in a recombinant yeast host cell, the polypeptides (including the enzymes) described herein are encoded on one or more heterologous nucleic acid molecule. In some embodiments, polypeptides (including the enzymes) described herein are encoded on one heterologous nucleic acid molecule, two heterologous nucleic acid molecules or copies, three heterologous nucleic acid molecules or copies, four heterologous nucleic acid molecules or copies, five heterologous nucleic acid molecules or copies, six heterologous nucleic acid molecules or copies, seven heterologous nucleic acid molecules or copies, or eight or more heterologous nucleic acid molecules or copies. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that was removed from the organism (which can, in some embodiments, be a source organism) and subsequently reintroduced into the organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or polypeptide) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When an heterologous nucleic acid molecule is present in the recombinant yeast host cell, it can be integrated in the yeast host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the host cell's genome. In such embodiment, the nucleic acid molecule can be stable and self-replicating.

In some embodiments, heterologous nucleic acid molecules which can be introduced into the recombinant yeast host cells are codon-optimized with respect to the intended recipient recombinant yeast host cell. As used herein, the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure can comprise a coding region for the one or more polypeptides (including enzymes) to be expressed by the recombinant host cell and/or one or more regulatory regions. A DNA or RNA “coding region” is a DNA or RNA molecule which is transcribed and/or translated into a polypeptide in a cell in vitro or in vivo when placed under the control of appropriate regulatory sequences. “Regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced and optionally maintained in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

In the heterologous nucleic acid molecule described herein, the promoter and the nucleic acid molecule coding for the one or more polypeptides (including enzymes) can be operatively linked to one another. In the context of the present disclosure, the expressions “operatively linked” or “operatively associated” refers to fact that the promoter is physically associated to the nucleotide acid molecule coding for the one or more enzyme in a manner that allows, under certain conditions, for expression of the one or more enzyme from the nucleic acid molecule. In an embodiment, the promoter can be located upstream (5′) of the nucleic acid sequence coding for the one or more enzyme. In still another embodiment, the promoter can be located downstream (3′) of the nucleic acid sequence coding for the one or more enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more enzyme. The promoters can be located, in view of the nucleic acid molecule coding for the one or more polypeptide, upstream, downstream as well as both upstream and downstream.

The expression “promoter” refers to a DNA fragment capable of controlling the expression of a coding sequence or functional RNA. The term “expression” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

The promoter can be heterologous to the nucleic acid molecule encoding the one or more polypeptides. The promoter can be heterologous or derived from a strain being from the same genus or species as the recombinant yeast host cell. In an embodiment, the promoter is derived from the same genus or species of the yeast host cell and the heterologous polypeptide is derived from different genus that the host cell.

In an embodiment, the present disclosure concerns the expression of one or more polypeptide (including an enzyme), a variant thereof or a fragment thereof in a recombinant host cell. A variant comprises at least one amino acid difference when compared to the amino acid sequence of the native polypeptide and exhibits a biological activity substantially similar to the native polypeptide. The polypeptide “variants” have at least 50%, 55%, 60%, 65%, 70%, 75%, 80%, 85%, 90%, 95%, 96%, 97%, 98% or 99% identity to the polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE™ bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the enzyme.

The polypeptide can be a fragment of polypeptide or fragment of a variant polypeptide. A polypeptide fragment comprises at least one less amino acid residue when compared to the amino acid sequence of the possesses and still possess a biological activity substantially similar to the native full-length polypeptide or polypeptide variant. Polypeptide “fragments” have at least at least 100, 200, 300, 400, 500 or more consecutive amino acids of the polypeptide or the polypeptide variant. The polypeptide “fragments” have at least 50%, 55%, 60%, 65%, 70%, 75%, 80%, 85%, 90%, 95%, 96%, 97%, 98% or 99% identity to the polypeptide described herein. In some embodiments, fragments of the polypeptides can be employed for producing the corresponding full-length enzyme by peptide synthesis. Therefore, the fragments can be employed as intermediates for producing the full-length polypeptides.

In some additional embodiments, the present disclosure also provides expressing a polypeptide encoded by a gene ortholog of a gene known to encode the polypeptide. A “gene ortholog” is understood to be a gene in a different species that evolved from a common ancestral gene by speciation. In the context of the present disclosure, a gene ortholog encodes polypeptide exhibiting a biological activity substantially similar to the native polypeptide.

In some further embodiments, the present disclosure also provides expressing a polypeptide encoded by a gene paralog of a gene known to encode the polypeptide. A “gene paralog” is understood to be a gene related by duplication within the genome. In the context of the present disclosure, a gene paralog encodes a polypeptide that could exhibit additional biological functions when compared to the native polypeptide.

In the context of the present disclosure, the recombinant/native/further yeast host cell is a yeast. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, Saccharomyces cerevisiae, Saccharomyces bulderi, Saccharomyces barnetti, Saccharomyces exiguus, Saccharomyces uvarum, Saccharomyces diastaticus, Kluyveromyces lactis, Kluyveromyces marxianus or Kluyveromyces fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the recombinant yeast host cell is from the genus Saccharomyces and, in some additional embodiments, from the species Saccharomyces cerevisiae.

Since the recombinant yeast host cell can be used for the fermentation of a biomass and the generation of fermentation product, it is contemplated herein that it has the ability to convert a biomass into a fermentation product without the including the additional genetic modifications described herein. In an embodiment, the recombinant yeast host cell has the ability to convert starch into ethanol during fermentation, as it is described below.

### Genetic Modification for Downregulating NADPH Production

In order to create increased glycolytic flux, there needs to be sufficient cofactors and/or reactants required by glycolysis. In the context of the present disclosure, downregulating a first metabolic pathway for conversion of NADP⁺ to NADPH and upregulating a second metabolic pathway for conversion of NADP⁺ to NADPH, comprises reducing the consumption of NADP⁺ by the first metabolic pathway and thereby making it available for the second metabolic pathway. Without wishing to be bound to theory, the second metabolic pathway favors the production of one or more fermented products (such as ethanol) which results in less substrate availability for the production of another fermented product, such as glycerol. In some embodiments, the first pathway is the pentose phosphate pathway, also known as the oxidative pentose phosphate pathway or the oxidative stage of the pentose phosphate pathway. In one embodiment, the first pathway is the cytosolic oxidative pentose phosphate pathway. In one embodiment, the first pathway is the hexose monophosphate shunt (or cycle). In one embodiment, the first pathway is the phosphogluconate pathway.

The present disclosure provides for a first genetic modification comprising inactivation of at least one first native gene, for downregulating the first pathway. In some embodiments, a recombinant yeast host cell is provided having native sources of NADPH regeneration downregulated with respect to this first pathway (when compared to a corresponding yeast host cell lacking the first genetic modification). In some further embodiments, the recombinant yeast host cell has at least one inactivated gene encoding for a polypeptide capable of producing NADPH.

There are three reactions during the oxidative stage of the pentose phosphate pathway. The first reaction is the oxidation of glucose-6-phosphate into 6-phosphogluconate by glucose-6-phosphate dehydrogenase (ZWF1) using NADP⁺ as a cofactor. The second reaction is the conversion of 6-phosphogluconolactone into 6-phosphogluconate by gluconolactonase. The third reaction is the oxidization of 6-phosphogluconate into ribulose-5-phosphate by 6-phosphogluconate dehydrogenase (GND1 and/or GND2) using NADP⁺ as a cofactor. Most of a cell's NADP⁺ consumption or NADPH regeneration comes from this first reaction by ZWF1. As such, in an embodiment, the first genetic modification comprises the inactivation of the gene encoding ZWF1.

Alternatively or in combination, the first genetic modification can include the inactivation of another gene encoding a polypeptide capable of producing NADPH. For example, the first genetic modification includes the inactivation of at least one of the following native genes: glucose-6-phosphate dehydrogenase (ZWF1), 6-phosphogluconate dehydrogenase (GND1 and/or GND2), NAD(P) aldehyde dehydrogenase (ALD6) and/or NADP dependent isocitrate dehydrogenase (IDP1, IDP2 and/or IDP3). For example, a number of other enzymes also consumes NADP⁺ to regenerate NADPH, and are summarized in Table 1. As such, in still another embodiment, the first genetic modification comprises the inactivation of a gene encoding one or more polypeptide as listed in Table 1.

TABLE 1
Embodiments enzymes that convert NADP⁺ to NADPH.
The amino acid sequence provided refers to the
Saccharomyces cerevisiae sequence.

| Gene | Enzyme | SEQ ID NO |
|---|---|---|
| ZWF1 | Glucose-6-phosphate dehydrogenase | 3 |
| GND1 | 6-phosphogluconate dehydrogenase | 4 |
| GND2 | 6-phosphogluconate dehydrogenase | 5 |
| ALD6 | NAD(P) aldehyde dehydrogenase | 6 |
| IDP1 | NADP dependent isocitrate dehydrogenase | 7 |
| IDP2 | NADP dependent isocitrate dehydrogenase | 8 |
| IDP3 | NADP dependent isocitrate dehydrogenase | 9 |

In one embodiment, the at least one first native gene comprises a zwf1 gene, an ortholog of the zwf1 gene or a paralog of the zwf1 gene. The zwf1 gene encodes a polypeptide having glucose-6-phosphate dehydrogenase activity. In one embodiment, the polypeptide having glucose-6-phosphate dehydrogenase activity has the amino acid sequence of SEQ ID NO: 3; is a variant of SEQ ID NO: 3, or is a fragment of SEQ ID NO: 3.

In one embodiment, the at least one first native gene comprises a gnd1 gene, an ortholog of the gnd1 gene or a paralog of the gnd1 gene. The gnd1 gene encodes a polypeptide having 6-phosphogluconate dehydrogenase activity. In one embodiment, the polypeptide having 6-phosphogluconate dehydrogenase activity has the amino acid sequence of SEQ ID NO: 4; is a variant of SEQ ID NO: 4, or is a fragment of SEQ ID NO: 4.

In one embodiment, the at least one first native gene comprises a gnd2 gene, an ortholog of the gnd2 gene or a paralog of the gnd2 gene. The gnd2 gene encodes a polypeptide having 6-phosphogluconate dehydrogenase activity. In one embodiment, the polypeptide having 6-phosphogluconate dehydrogenase activity has the amino acid sequence of SEQ ID NO: 5; is a variant of SEQ ID NO: 5, or is a fragment of SEQ ID NO: 5.

In one embodiment, the at least one first native gene comprises a ald6 gene, an ortholog of the ald6 gene or a paralog of the ald6 gene. The ald6 gene encodes a polypeptide having aldehyde dehydrogenase activity. In one embodiment, the polypeptide having aldehyde dehydrogenase activity has the amino acid sequence of SEQ ID NO: 6; is a variant of SEQ ID NO: 6, or is a fragment of SEQ ID NO: 6.

In one embodiment, the at least one first native gene comprises a idp1 gene, an ortholog of the idp1 gene or a paralog of the idp1 gene. The idp1 gene encodes a polypeptide having isocitrate dehydrogenase activity. In one embodiment, the polypeptide having isocitrate dehydrogenase activity has the amino acid sequence of SEQ ID NO: 7; is a variant of SEQ ID NO: 7, or is a fragment of SEQ ID NO: 7.

In one embodiment, the at least one first native gene comprises a idp2 gene, an ortholog of the idp2 gene or a paralog of the idp2 gene. The idp2 gene encodes a polypeptide having isocitrate dehydrogenase activity. In one embodiment, the polypeptide having isocitrate dehydrogenase activity has the amino acid sequence of SEQ ID NO: 8; is a variant of SEQ ID NO: 8, or is a fragment of SEQ ID NO: 8.

In one embodiment, the at least one first native gene comprises a ipd3 gene, an ortholog of the ipd3 gene or a paralog of the ipd3 gene. The ipd3 gene encodes a polypeptide having isocitrate dehydrogenase activity. In one embodiment, the polypeptide having isocitrate dehydrogenase activity has the amino acid sequence of SEQ ID NO: 9; is a variant of SEQ ID NO: 9, or is a fragment of SEQ ID NO: 9.

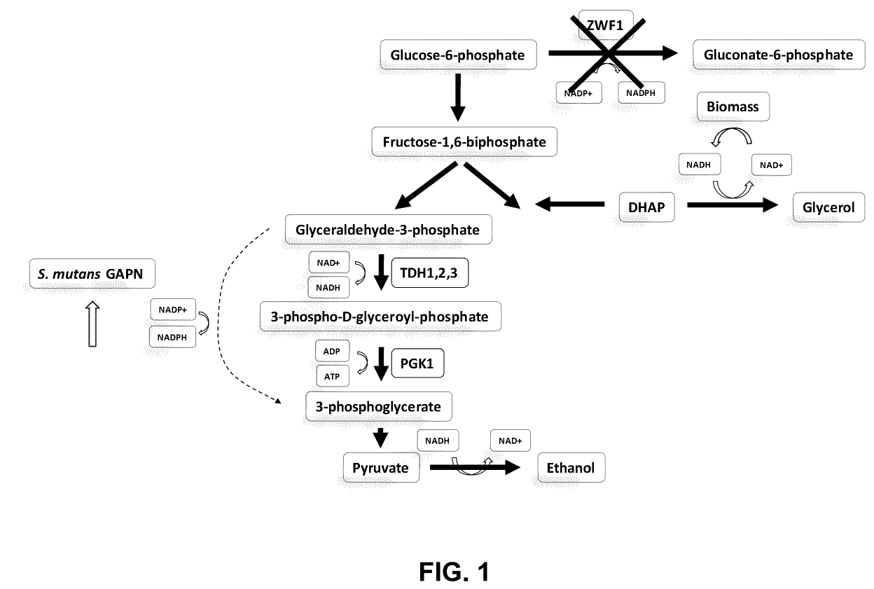

In one embodiment as outlined in FIG. 1, it has been found that combining the expression of the GAPN gene and inactivating the zwf1 gene (zwf1Δ) provides an effective way to increase glycolytic flux, with GAPN acting as a surrogate NADPH generator. When expressed in zwf1Δ cells, GAPN is able to regenerate NADPH from NADP⁺ by catalyzing the reaction of glyceraldehyde-3-phosphate to 3-phosphoglycerate, thereby adding glycolytic flux towards pyruvate. This additional activity in combination with zwf1Δ maintains the integrity and functionality of native glycolytic pathways while reducing glycerol production and increasing ethanol yield. Additionally, the zwf1Δ-GAPN pathway does not result in the production of toxic intermediates, by-products, or end products, reducing the risk of autotoxicity in engineered cells.

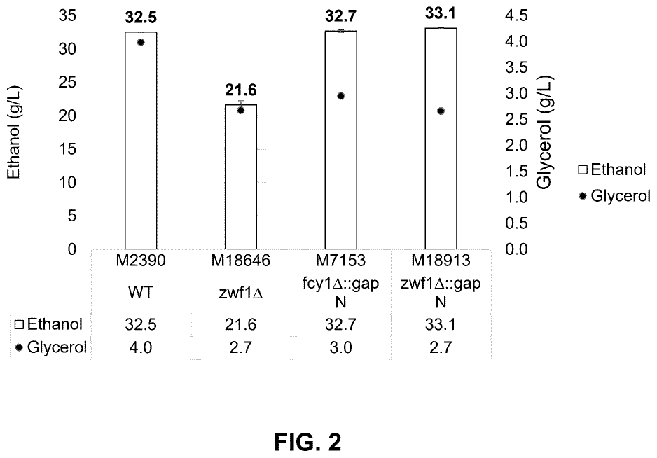

In some embodiments, this zwf1Δ-GAPN pathway does not require any modifications to the glycerol-3-phosphate dehydrogenase genes (GPD), or the glycerol-3-phosphate phosphatase genes (GPP). As shown in FIG. 2, fermentation with recombinant yeast host cells having this zwf1A-GAPN pathway exhibits increased ethanol yield compared to wild type yeast. At the same time, this zwf1Δ-GAPN recombinant yeast host cell also significantly decreasedGAPN introduced by zwf1 still active (fcy1Δ-GAPN).

In some embodiments, the first genetic modification comprising inactivation of a first native gene, and the second genetic modification are employed dependent on each other. For example, the second genetic modification can be made in such a way that the heterologous nucleic acid molecule comprising a glyceraldehyde-3-phosphate dehydrogenase is positioned to be under the control of the first promoter of the first native gene. As such, by introducing the heterologous nucleic acid molecule inside the first native gene, the first native gene is inactivated. In one embodiment, the heterologous nucleic acid molecule comprising a glyceraldehyde-3-phosphate dehydrogenase is in an open reading frame of the first native gene.

In one embodiment, the first genetic modification comprising zwf1Δ and the second genetic modification comprising GAPN are employed dependent on each other. In one embodiment, the heterologous nucleic acid molecule comprising the GAPN gene is positioned to be placed under the control of the first promoter of the native zwf1 gene. In one embodiment, the heterologous nucleic acid molecule comprising the GAPN gene is in an open reading frame of the native zwf1 gene.

### Non-Phosphorylating Glyceraldehyde-3-Phosphate Dehydrogenase

In the context of the present disclosure, downregulating a first pathway for conversion of NADP⁺ to NADPH and upregulating a second pathway for conversion of NADP⁺ to NADPH, comprises preferentially providing NADP⁺ to the second pathway. In some embodiments, the second pathway is a glycolytic pathway. In one embodiment, increased glycolytic flux results in reduced glycerol formation and increased ethanol titers during yeast fermentation. The present disclosure provides for a second genetic modification comprising overexpression of a heterologous polypeptide, for upregulating the second pathway. In some embodiments, the second genetic modification comprises the introduction of a heterologous nucleic acid molecule in the recombinant yeast host cell. In some embodiments, the heterologous nucleic acid molecule encodes a glyceraldehyde-3-phosphate dehydrogenase. As shown in FIG. 1, in some additional embodiments, the glyceraldehyde-3-phosphate dehydrogenase bypasses the reactions catalyzed by TDH1, THD2, TDH3 and PGK1 in the first metabolic pathway. In Saccharomyces cerevisiae, the enzyme TDH1 can have the amino acid of SEQ ID NO: 22, the enzyme TDH2 can have the amino acid sequence of SEQ ID NO: 23 and/or the enzyme TDH3 can have the amino acid sequence of SEQ ID NO: 24. In one embodiment, the heterologous nucleic acid molecule encodes GAPN.

Introducing and expressing a heterologous glyceraldehyde-3-phosphate dehydrogenase in the recombinant yeast host cell as described herein allows the catalysis of the reaction of glyceraldehyde-3-phosphate to 3-phosphoglycerate in glycolysis, using NADP⁺ as a cofactor. In some embodiments, regeneration of NADPH and/or NADH by way a glycolytic pathway using glyceraldehyde-3-phosphate also improves ethanol production and reduces glycerol production.

The present disclosure provides for a recombinant yeast host cell expressing heterologous glyceraldehyde-3-phosphate dehydrogenase. This enzyme catalyzes the conversion of glyceraldehyde-3-phosphate to 3-phosphoglycerate, using NADP⁺ as a co-factor. In some embodiments, the glyceraldehyde-3-phosphate could also use NAD⁺ as a cofactor. The glyceraldehyde-3-phosphate dehydrogenase is a non-phosphorylating glyceraldehyde-3-phosphate dehydrogenase, e.g., it is incapable of mediating a phosphorylation reaction. In some embodiments, the glyceraldehyde-3-phosphate dehydrogenase is of enzyme commission (EC) class 1.2.1, however it excludes the enzymes capable of mediating a phosphorylating reaction. The glyceraldehyde-3-phosphate dehydrogenase of the present disclosure specifically exclude enzymes capable of directly using or generating of 3-phospho-D-glyceroyl phosphate, such as enzymes of EC 1.2.1.13. Enzymes of EC 1.2.1.13 catalyze the following reaction:

$$D - glyceraldehyde 3 - phosphate + phosphate + {NADP}^{+} \text{<=>} 3 - phospho - D - glyceroyl phosphate + NADPH$$

In one embodiment, the glyceraldehyde-3-phosphate dehydrogenase is NADP⁺ dependent (EC1.2.1.9) and allows the conversion of NADP⁺ to NADPH. Enzymes of EC1.2.1.9 can only use NADP⁺ as a cofactor.

In one embodiment, the glyceraldehyde-3-phosphate dehydrogenase is bifunctional NADP⁺/NAD⁺ dependent (EC1.2.1.90) and allows the conversion of NADP⁺ to NADPH and/or NAD⁺ to NAD⁺. Enzymes of EC1.2.1.90 can use NADP⁺ or NAD⁺ as a cofactor. In some embodiments, glyceraldehyde-3-phosphate dehydrogenase uses NADP⁺ and/or NAD⁺ as a cofactor. In one embodiment, the glyceraldehyde-3-phosphate dehydrogenase is encoded by a GAPN gene. In one embodiment, the glyceraldehyde-3-phosphate dehydrogenase is GAPN.

In the context of the present disclosure, the second genetic modification can include the introduction of one or more copies of an heterologous nucleic acid molecule encoding the glyceraldehyde-3-phosphate dehydrogenase.

In some embodiments, the glyceraldehyde-3-phosphate dehydrogenase can be derived from a bacteria, for example, from the genus Streptococcus and, in some instances, from the species Streptococcus mutans. The glyceraldehyde-3-phosphate dehydrogenase can be encoded by the GAPN gene from Streptococcus mutans, or a GAPN gene ortholog, or a GAPN gene paralog. In an embodiment, the GAPN gene comprises the nucleic acid sequence of SEQ ID NO: 1, is a variant of the nucleic acid sequence of SEQ ID NO: 1 or is a fragment of the nucleic acid sequence of SEQ ID NO: 1. In an embodiment, the GAPN has the amino acid sequence of SEQ ID NO: 2, is a variant of the amino acid of SEQ ID NO: 2 or is a fragment of SEQ ID NO: 2.

In some embodiments, the glyceraldehyde-3-phosphate dehydrogenase can be derived from a bacteria, for example, from the genus Lactobacillus and, in some instances, from the species Lactobacillus delbrueckii. The glyceraldehyde-3-phosphate dehydrogenase can be encoded by the GAPN gene from Lactobacillus delbrueckii, or a GAPN gene ortholog, or a GAPN gene paralog. In an embodiment, the GAPN gene comprises the nucleic acid sequence of SEQ ID NO: 46, is a variant of the nucleic acid sequence of SEQ ID NO: 46 or is a fragment of the nucleic acid sequence of SEQ ID NO: 46. In an embodiment, the GAPN has the amino acid sequence of SEQ ID NO: 47, is a variant of the amino acid of SEQ ID NO: 47 or is a fragment of SEQ ID NO: 47.

In some embodiments, the glyceraldehyde-3-phosphate dehydrogenase can be derived from a bacteria, for example, from the genus Streptococcus and, in some instances, from the species Streptococcus thermophilus. The glyceraldehyde-3-phosphate dehydrogenase can be encoded by the GAPN gene from Streptococcus thermophilus, or a GAPN gene ortholog, or a GAPN gene paralog. In an embodiment, the GAPN gene comprises the nucleic acid sequence of SEQ ID NO: 48, is a variant of the nucleic acid sequence of SEQ ID NO: 48 or is a fragment of the nucleic acid sequence of SEQ ID NO: 48. In an embodiment, the GAPN has the amino acid sequence of SEQ ID NO: 49, is a variant of the amino acid of SEQ ID NO: 49 or is a fragment of SEQ ID NO: 49.

In some embodiments, the glyceraldehyde-3-phosphate dehydrogenase can be derived from a bacteria, for example, from the genus Streptococcus and, in some instances, from the species Streptococcus macacae. The glyceraldehyde-3-phosphate dehydrogenase can be encoded by the GAPN gene from Streptococcus macacae, or a GAPN gene ortholog, or a GAPN gene paralog. In an embodiment, the GAPN gene comprises the nucleic acid sequence of SEQ ID NO: 50, is a variant of the nucleic acid sequence of SEQ ID NO: 50 or is a fragment of the nucleic acid sequence of SEQ ID NO: 50. In an embodiment, the GAPN has the amino acid sequence of SEQ ID NO: 51, is a variant of the amino acid of SEQ ID NO: 51 or is a fragment of SEQ ID NO: 51.

In some embodiments, the glyceraldehyde-3-phosphate dehydrogenase can be derived from a bacteria, for example, from the genus Streptococcus and, in some instances, from the species Streptococcus hyointestinalis. The glyceraldehyde-3-phosphate dehydrogenase can be encoded by the GAPN gene from Streptococcus hyointestinalis, or a GAPN gene ortholog, or a GAPN gene paralog. In an embodiment, the GAPN gene comprises the nucleic acid sequence of SEQ ID NO: 52, is a variant of the nucleic acid sequence of SEQ ID NO: 52 or is a fragment of the nucleic acid sequence of SEQ ID NO: 52. In an embodiment, the GAPN has the amino acid sequence of SEQ ID NO: 53, is a variant of the amino acid of SEQ ID NO: 53 or is a fragment of SEQ ID NO: 53.

In some embodiments, the glyceraldehyde-3-phosphate dehydrogenase can be derived from a bacteria, for example, from the genus Streptococcus and, in some instances, from the species Streptococcus urinalis. The glyceraldehyde-3-phosphate dehydrogenase can be encoded by the GAPN gene from Streptococcus urinalis, or a GAPN gene ortholog, or a GAPN gene paralog. In an embodiment, the GAPN gene comprises the nucleic acid sequence of SEQ ID NO: 54, is a variant of the nucleic acid sequence of SEQ ID NO: 54 or is a fragment of the nucleic acid sequence of SEQ ID NO: 54. In an embodiment, the GAPN has the amino acid sequence of SEQ ID NO: 55, is a variant of the amino acid of SEQ ID NO: 55 or is a fragment of SEQ ID NO: 55.

In some embodiments, the glyceraldehyde-3-phosphate dehydrogenase can be derived from a bacteria, for example, from the genus Streptococcus and, in some instances, from the species Streptococcus canis. The glyceraldehyde-3-phosphate dehydrogenase can be encoded by the GAPN gene from Streptococcus canis, or a GAPN gene ortholog, or a GAPN gene paralog. In an embodiment, the GAPN gene comprises the nucleic acid sequence of SEQ ID NO: 56, is a variant of the nucleic acid sequence of SEQ ID NO: 56 or is a fragment of the nucleic acid sequence of SEQ ID NO: 56. In an embodiment, the GAPN has the amino acid sequence of SEQ ID NO: 57, is a variant of the amino acid of SEQ ID NO: 57 or is a fragment of SEQ ID NO: 57.

In some embodiments, the glyceraldehyde-3-phosphate dehydrogenase can be derived from a bacteria, for example, from the genus Streptococcus and, in some instances, from the species Streptococcus thoraltensis. The glyceraldehyde-3-phosphate dehydrogenase can be encoded by the GAPN gene from Streptococcus thoraltensis, or a GAPN gene ortholog, or a GAPN gene paralog. In an embodiment, the GAPN gene comprises the nucleic acid sequence of SEQ ID NO: 58, is a variant of the nucleic acid sequence of SEQ ID NO: 58 or is a fragment of the nucleic acid sequence of SEQ ID NO: 58. In an embodiment, the GAPN has the amino acid sequence of SEQ ID NO: 59, is a variant of the amino acid of SEQ ID NO: 59 or is a fragment of SEQ ID NO: 59.

In some embodiments, the glyceraldehyde-3-phosphate dehydrogenase can be derived from a bacteria, for example, from the genus Streptococcus and, in some instances, from the species Streptococcus dysgalactiae. The glyceraldehyde-3-phosphate dehydrogenase can be encoded by the GAPN gene from Streptococcus dysgalactiae, or a GAPN gene ortholog, or a GAPN gene paralog. In an embodiment, the GAPN gene comprises the nucleic acid sequence of SEQ ID NO: 60, is a variant of the nucleic acid sequence of SEQ ID NO: 60 or is a fragment of the nucleic acid sequence of SEQ ID NO: 60. In an embodiment, the GAPN has the amino acid sequence of SEQ ID NO: 61, is a variant of the amino acid of SEQ ID NO: 61 or is a fragment of SEQ ID NO: 61.

In some embodiments, the glyceraldehyde-3-phosphate dehydrogenase can be derived from a bacteria, for example, from the genus Streptococcus and, in some instances, from the species Streptococcus pyogenes. The glyceraldehyde-3-phosphate dehydrogenase can be encoded by the GAPN gene from Streptococcus pyogenes, or a GAPN gene ortholog, or a GAPN gene paralog. In an embodiment, the GAPN gene comprises the nucleic acid sequence of SEQ ID NO: 71, is a variant of the nucleic acid sequence of SEQ ID NO: 71 or is a fragment of the nucleic acid sequence of SEQ ID NO: 71. In an embodiment, the GAPN has the amino acid sequence of SEQ ID NO: 72, is a variant of the amino acid of SEQ ID NO: 72 or is a fragment of SEQ ID NO: 72.

In some embodiments, the glyceraldehyde-3-phosphate dehydrogenase can be derived from a bacteria, for example, from the genus Streptococcus and, in some instances, from the species Streptococcus ictaluri. The glyceraldehyde-3-phosphate dehydrogenase can be encoded by the GAPN gene from Streptococcus ictaluri, or a GAPN gene ortholog, or a GAPN gene paralog. In an embodiment, the GAPN gene comprises the nucleic acid sequence of SEQ ID NO: 73, is a variant of the nucleic acid sequence of SEQ ID NO: 73 or is a fragment of the nucleic acid sequence of SEQ ID NO: 73. In an embodiment, the GAPN has the amino acid sequence of SEQ ID NO: 74, is a variant of the amino acid of SEQ ID NO: 74 or is a fragment of SEQ ID NO: 74.

In some embodiments, the glyceraldehyde-3-phosphate dehydrogenase can be derived from a bacteria, for example, from the genus Clostridium and, in some instances, from the species Clostridium perfringens. The glyceraldehyde-3-phosphate dehydrogenase can be encoded by the GAPN gene from Clostridium perfringens, or a GAPN gene ortholog, or a GAPN gene paralog. In an embodiment, the GAPN gene comprises the nucleic acid sequence of SEQ ID NO: 75, is a variant of the nucleic acid sequence of SEQ ID NO: 75 or is a fragment of the nucleic acid sequence of SEQ ID NO: 75. In an embodiment, the GAPN has the amino acid sequence of SEQ ID NO: 76, is a variant of the amino acid of SEQ ID NO: 76 or is a fragment of SEQ ID NO: 76.

In some embodiments, the glyceraldehyde-3-phosphate dehydrogenase can be derived from a bacteria, for example, from the genus Clostridium and, in some instances, from the species Clostridium chromiireducens. The glyceraldehyde-3-phosphate dehydrogenase can be encoded by the GAPN gene from Clostridium chromiireducens, or a GAPN gene ortholog, or a GAPN gene paralog. In an embodiment, the GAPN gene comprises the nucleic acid sequence of SEQ ID NO: 77, is a variant of the nucleic acid sequence of SEQ ID NO: 77 or is a fragment of the nucleic acid sequence of SEQ ID NO: 77. In an embodiment, the GAPN has the amino acid sequence of SEQ ID NO: 78, is a variant of the amino acid of SEQ ID NO: 78 or is a fragment of SEQ ID NO: 78.

In some embodiments, the glyceraldehyde-3-phosphate dehydrogenase can be derived from a bacteria, for example, from the genus Clostridium and, in some instances, from the species Clostridium botulinum. The glyceraldehyde-3-phosphate dehydrogenase can be encoded by the GAPN gene from Clostridium botulinum, or a GAPN gene ortholog, or a GAPN gene paralog. In an embodiment, the GAPN gene comprises the nucleic acid sequence of SEQ ID NO: 79, is a variant of the nucleic acid sequence of SEQ ID NO: 79 or is a fragment of the nucleic acid sequence of SEQ ID NO: 79. In an embodiment, the GAPN has the amino acid sequence of SEQ ID NO: 80, is a variant of the amino acid of SEQ ID NO: 80 or is a fragment of SEQ ID NO: 80.

In some embodiments, the glyceraldehyde-3-phosphate dehydrogenase can be derived from a bacteria, for example, from the genus Bacillus and, in some instances, from the species Bacillus cereus. The glyceraldehyde-3-phosphate dehydrogenase can be encoded by the GAPN gene from Bacillus cereus, or a GAPN gene ortholog, or a GAPN gene paralog. In an embodiment, the GAPN gene comprises the nucleic acid sequence of SEQ ID NO: 81, is a variant of the nucleic acid sequence of SEQ ID NO: 81 or is a fragment of the nucleic acid sequence of SEQ ID NO: 81. In an embodiment, the GAPN has the amino acid sequence of SEQ ID NO: 82, is a variant of the amino acid of SEQ ID NO: 82 or is a fragment of SEQ ID NO: 82.

In some embodiments, the glyceraldehyde-3-phosphate dehydrogenase can be derived from a bacteria, for example, from the genus Bacillus and, in some instances, from the species Bacillus anthracis. The glyceraldehyde-3-phosphate dehydrogenase can be encoded by the GAPN gene from Bacillus anthracis, or a GAPN gene ortholog, or a GAPN gene paralog. In an embodiment, the GAPN gene comprises the nucleic acid sequence of SEQ ID NO: 83, is a variant of the nucleic acid sequence of SEQ ID NO: 83 or is a fragment of the nucleic acid sequence of SEQ ID NO: 83. In an embodiment, the GAPN has the amino acid sequence of SEQ ID NO: 84, is a variant of the amino acid of SEQ ID NO: 84 or is a fragment of SEQ ID NO: 84.

In some embodiments, the glyceraldehyde-3-phosphate dehydrogenase can be derived from a bacteria, for example, from the genus Bacillus and, in some instances, from the species Bacillus thuringiensis. The glyceraldehyde-3-phosphate dehydrogenase can be encoded by the GAPN gene from Bacillus thuringiensis, or a GAPN gene ortholog, or a GAPN gene paralog. In an embodiment, the GAPN gene comprises the nucleic acid sequence of SEQ ID NO: 85, is a variant of the nucleic acid sequence of SEQ ID NO: 85 or is a fragment of the nucleic acid sequence of SEQ ID NO: 85. In an embodiment, the GAPN has the amino acid sequence of SEQ ID NO: 86, is a variant of the amino acid of SEQ ID NO: 86 or is a fragment of SEQ ID NO: 86.

In some embodiments, the glyceraldehyde-3-phosphate dehydrogenase can be derived from a bacteria, for example, from the genus Pyrococcus and, in some instances, from the species Pyrococcus furiosus. The glyceraldehyde-3-phosphate dehydrogenase can be encoded by the GAPN gene from Pyrococcus furiosus, or a GAPN gene ortholog, or a GAPN gene paralog. In an embodiment, the GAPN gene comprises the nucleic acid sequence of SEQ ID NO: 87, is a variant of the nucleic acid sequence of SEQ ID NO: 87 or is a fragment of the nucleic acid sequence of SEQ ID NO: 87. In an embodiment, the GAPN has the amino acid sequence of SEQ ID NO: 88, is a variant of the amino acid of SEQ ID NO: 88 or is a fragment of SEQ ID NO: 88. Embodiments of glyceraldehyde-3-phosphate dehydrogenase can also be derived, without limitation, from the following (the number in brackets correspond to the Gene ID number): Triticum aestivum (543435); Streptococcus mutans (1028095); Streptococcus agalactiae (1013627); Streptococcus pyogenes (901445); Clostridioides difficile (4913365); Mycoplasma mycoides subsp. mycoides SC str. (2744894); Streptococcus pneumoniae (933338); Streptococcus sanguinis (4807521); Acinetobacter pittii (11638070); Clostridium botulinum A str. (5185508); [Bacillus thuringiensis] serovar konkukian str. (2857794); Bacillus anthracis str. Ames (1088724); Phaeodactylum tricornutum (7199937); Emiliania huxleyi (17251102); Zea mays (542583); Helianthus annuus (110928814); Streptomyces coelicolor (1101118); Burkholderia pseudomallei (U.S. Pat. Nos. 3,097,058, 3,095,849); variants thereof as well as fragments thereof.

Additional embodiments of glyceraldehyde-3-phosphate dehydrogenase can also be derived, without limitation, from the following (the number in brackets correspond to the Pubmed Accession number): Streptococcus macacae (WP_003081126.1), Streptococcus hyointestinalis (WP_115269374.1), Streptococcus urinalis (WP_006739074.1), Streptococcus canis (WP_003044111.1), Streptococcus pluranimalium (WP_104967491.1), Streptococcus equi (WP_012678132.1), Streptococcus thoraltensis (WP_018380938.1), Streptococcus dysgalactiae (WP_138125971.1), Streptococcus halotolerans (WP_062707672.1), Streptococcus pyogenes (WP_136058687.1), Streptococcus ictaluri (WP_008090774.1), Clostridium perfringens (WP_142691612.1), Clostridium chromiireducens (WP_079442081.1), Clostridium botulinum (WP_012422907.1), Bacillus cereus (WP_000213623.1), Bacillus anthracis (WP_098340670.1), Bacillus thuringiensis (WP_087951472.1), Pyrococcus furiosus (WP_011013013.1) as well as variants thereof and fragments thereof.

In some embodiments, the glyceraldehyde-3-phosphate dehydrogenase encoded by the GAPN gene (GAPN) comprises the amino acid sequence of SEQ ID NO: 2, 47, 49, 51, 53, 55, 57, 59, or 61 is a variant of the amino acid sequence of SEQ ID NO: 2, 47, 49, 51, 53, 55, 57, 59, or 61 or is a fragment of the amino acid sequence of SEQ ID NO: 2, 47, 49, 51, 53, 55, 57, 59, or 61. In some embodiment, the glyceraldehyde-3-phosphate dehydrogenase is expressed intracellularly.

In the context of the present disclosure, GAPN include variants of the glyceraldehyde-3-phosphate dehydrogenase of SEQ ID NO: 2, 47, 49, 51, 53, 55, 57, 59, or 61 (also referred to herein as GAPN variants). A variant comprises at least one amino acid difference (substitution or addition) when compared to the amino acid sequence of the glyceraldehyde-3-phosphate dehydrogenase of SEQ ID NO: 2, 47, 49, 51, 53, 55, 57, 59, or 61. The GAPN variants do exhibit GAPN activity. In an embodiment, the variant GAPN exhibits at least 50%, 60%, 70%, 80%, 90%, 95%, 96%, 97%, 98% or 99% of the glyceraldehyde-3-phosphate dehydrogenase of SEQ ID NO: 2. The GAPN variants also have at least 70%, 80%, 85%, 90%, 95%, 96%, 97%, 98% or 99% identity to the amino acid sequence of SEQ ID NO: 2, 47, 49, 51, 53, 55, 57, 59, or 61. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE™ bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant GAPN described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant GAPN can also be a conservative variant or an allelic variant. As used herein, a conservative variant refers to alterations in the amino acid sequence that do not adversely affect the biological functions of GAPN. A substitution, insertion or deletion is said to adversely affect the polypeptide when the altered sequence prevents or disrupts a biological function associated with GAPN (e.g., glycolysis). For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of GAPN.

The present disclosure also provide fragments of the GAPN and variants described herein. A fragment comprises at least one less amino acid residue when compared to the amino acid sequence of the GAPN or variant and still possess the enzymatic activity of the full-length GAPN. In an embodiment, the GAPN fragment exhibits at least 50%, 60%, 70%, 80%, 90%, 95%, 96%, 97%, 98% or 99% of the full-length glyceraldehyde-3-phosphate dehydrogenase of SEQ ID NO: 2, 47, 49, 51, 53, 55, 57, 59, or 61. The GAPN fragments can also have at least 70%, 80%, 85%, 90%, 95%, 96%, 97%, 98% or 99% identity to the amino acid sequence of SEQ ID NO: 2, 47, 49, 51, 53, 55, 57, 59, or 61. The fragment can be, for example, a truncation of one or more amino acid residues at the amino-terminus, the carboxy terminus or both termini of GAPN or variant. Alternatively or in combination, the fragment can be generated from removing one or more internal amino acid residues. In an embodiment, the GAPN fragment has at least 100, 150, 200, 250, 300, 350, 400, 450 or more consecutive amino acids of GAPN or the variant.

The heterologous nucleic acid encoding the glyceraldehyde-3-phosphate dehydrogenase can be positioned in the open reading frame of the first native gene and can use the promoter of the first native gene to drive its expression.

Alternatively or in combination, the heterologous nucleic acid molecule encoding the glyceraldehyde-3-phosphate dehydrogenase can include an heterologous promoter. In the context of the present disclosure, the heterologous promoter controlling the expression of the heterologous nucleic acid molecule can be a constitutive promoter (such as, for example, tef2p (e.g., the promoter of the TEF2 gene), cwp2p (e.g., the promoter of the CWP2 gene), ssa1p (e.g., the promoter of the SSA1 gene), eno1p (e.g., the promoter of the ENO1 gene), hxk1 (e.g., the promoter of the HXK1 gene), pgi1p (e.g., the promotoer from the PGI1 gene), pfk1p (e.g., the promoter from the PFK1 gene), fba1p (e.g., the promoter from the FBA1 gene), gpm1p (e.g., the promoter from the GPM1 gene) and/or pgk1p (e.g., the promoter of the PGK1 gene). However, is some embodiments, it is preferable to limit the expression of the heterologous polypeptide. As such, the promoter controlling the expression of the heterologous glyceraldehyde-3-phosphate dehydrogenase can be an inducible or modulated promoters such as, for example, a glucose-regulated promoter (e.g., the promoter of the HXT7 gene (referred to as hxt7p)), a pentose phosphate pathway promoter (e.g., the promoter of the ZWF1 gene (zwf1p)) or a sulfite-regulated promoter (e.g., the promoter of the GPD2 gene (referred to as gpd2p) or the promoter of the FZF1 gene (referred to as the fzf1p)), the promoter of the SSU1 gene (referred to as ssu1p), the promoter of the SSU1-r gene (referred to as ssur1-rp). In an embodiment, the promoter is an anaerobic-regulated promoters, such as, for example tdh1p (e.g., the promoter of the TDH1 gene), pau5p (e.g., the promoter of the PAU5 gene), hor7p (e.g., the promoter of the HOR7 gene), adh1p (e.g., the promoter of the ADH1 gene), tdh2p (e.g., the promoter of the TDH2 gene), tdh3p (e.g., the promoter of the tdh3 gene), gpd1p (e.g., the promoter of the GPD1 gene), cdc19p (e.g., the promoter of the CDCl9 gene), eno2p (e.g., the promoter of the ENO2 gene), pdc1p (e.g., the promoter of the PDC1 gene), hxt3p (e.g., the promoter of the HXT3 gene), dan1 (e.g., the promoter of the DAN1 gene) and tpi1p (e.g., the promoter of the TPI1 gene). In yet another embodiment, the promoter is a cytochrome c/mitochondrial electron transport chain promoter, such as, for example, the cyc1p (e.g., the promoter of the CYC1 gene) and/or the qcr8p (e.g., the promoter of the QCR8 gene). In an embodiment, the heterologous promoter is gpd1p, e.g., the promoter of the GPD1 gene. In another embodiment, the heterologous promoter is zwf1, e.g., the promoter of the ZWF1 gen. One or more promoters can be used to allow the expression of each heterologous polypeptides in the recombinant yeast host cell.

In an embodiment, the second polypeptide is expressed intracellularly and, if necessary, the signal sequence is removed from the native sequence.

### Characterization and Comparison of Glyceraldehyde-3-Phosphate Dehydrogenases

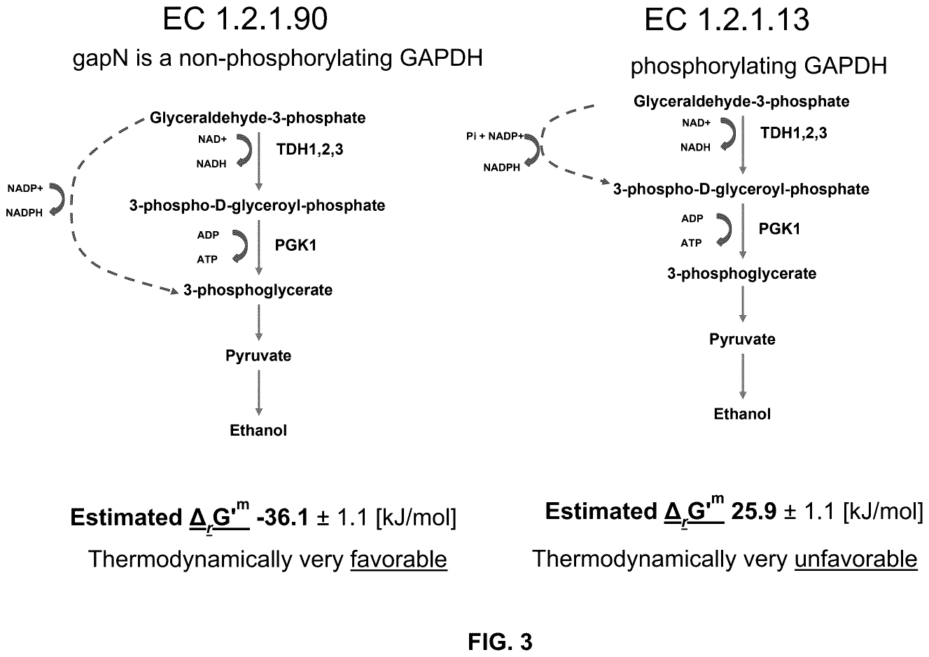

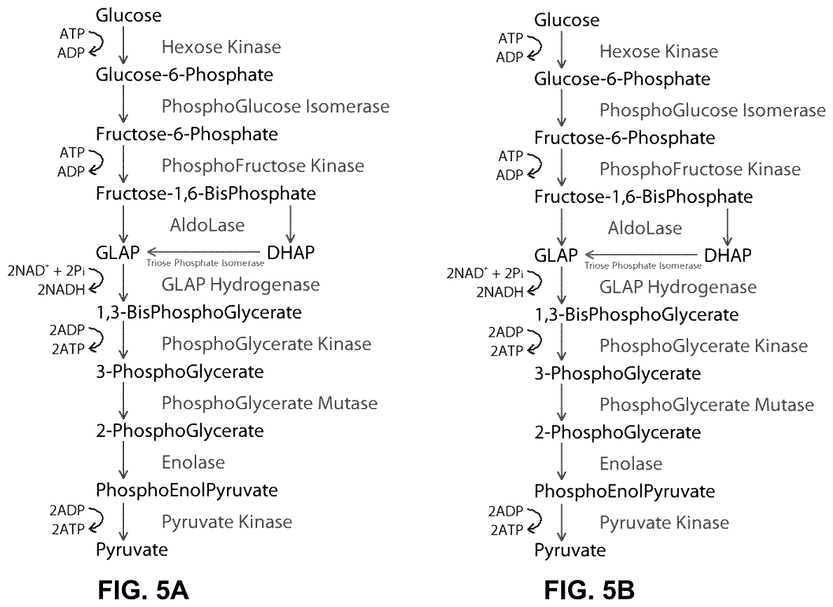

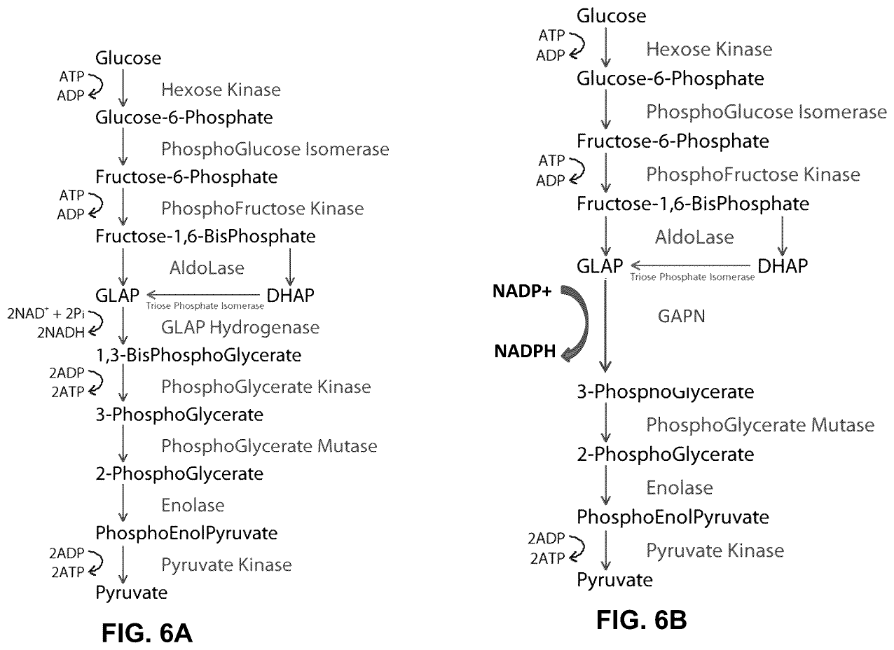

As it is known in the art, glyceraldehyde-3-phosphate dehydrogenases (GAPDH) can have phosphorylating activity or lack phosphorylating activity (e.g., non-phosphorylating), and can also be NAD⁺- and/or NADP⁺-dependent (see for example, EC1.2.1.9, EC1.2.1.12, EC1.2.1.13, EC1.2.1.59, EC1.2.1.9). As shown in FIG. 3, GAPN is a NAPDH-dependent which lacks phosphorylating activity (e.g., non-phosphorylating) and catalyzes the reaction of glyceraldehyde-3-phosphate to 3-phosphoglycerate without generating any ATP (see FIG. 6). Since no ATP is generated, the GAPN-catalyzed reaction is thermodynamically very favorable. On the other hand, GDP1 is a NADP⁺ dependent phosphorylating GAPDH, and the glycolysis reaction generates two molecules of ATP when converting glyceraldehyde-3-phosphate to 3-phosphoglycerate (see FIG. 5). Since ATP will be generated, the GDP1 catalyzed reaction is not thermodynamically favorable. Similarly, NAD⁺ dependent phosphorylating GAPDH (EC 1.2.1.12) also generates ATP and is also thermodynamically unfavorable.

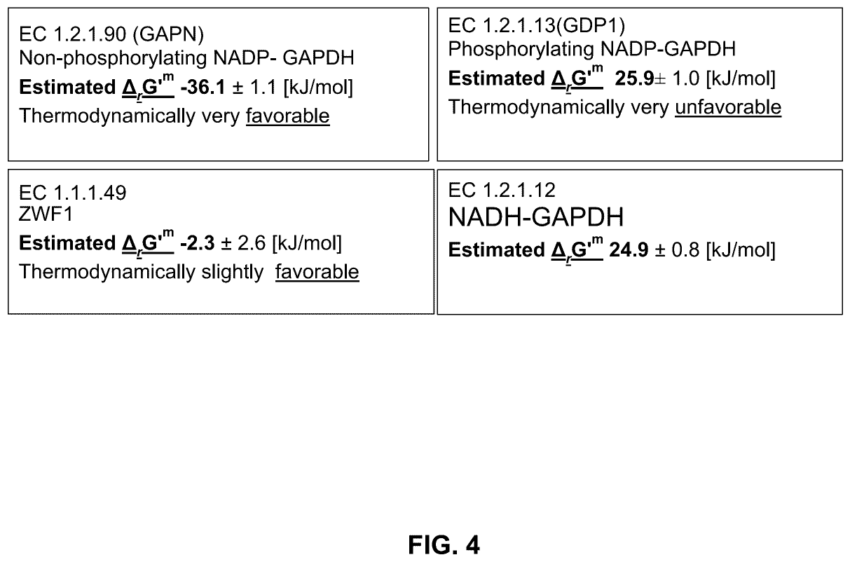

The thermodynamics of GAPN (EC1.2.1.9), GDP1 (EC1.2.1.13), and NAD⁺ dependent phosphorylating GAPDH (EC 1.2.1.12) are summarized in FIG. 4 and Table 2. As shown in Table 2, the inactivation of zwf1 also has a negative Gibbs Energy value. In a zwf1 knockout strain the loss of NADPH regeneration by zwf1 should be compensated by other enzymes. Furthermore, for optimal fermentation by a zwf1 knockout, GAPN-expressing strain, the regeneration rate of NADPH by GAPN should complement the regeneration rate of NADPH by zwf1.

TABLE 2
Estimated Gibbs Energy value of reactions
catalyzed by GAPN and Δzwf1.

| Enzyme | Estimated ΔᵣG′^{m} |
|---|---|
| GAPN (EC1.2.1.9) | −36.1 ± 1.1 kJ/mol |
| GDP1 (EC1.2.1.13) | 25.9 ± 1.0 kJ/mol |
| NAD⁺ dependent | 24.9 ± 0.8 kJ/mol |
| phosphorylating | |
| GAPDH | |
| (EC1.2.1.12) | |
| Δzwf1 | −2.3 ± 2.6 kJ/mol |

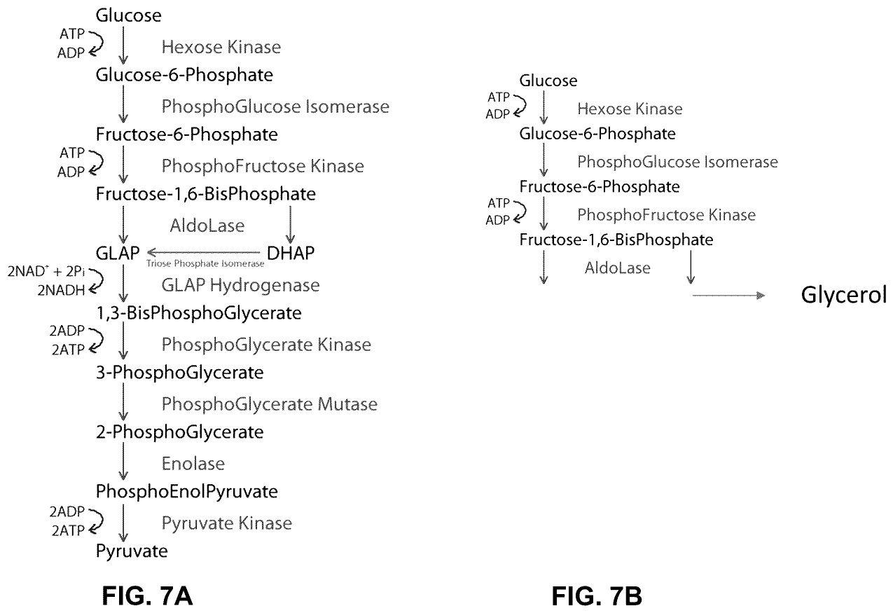

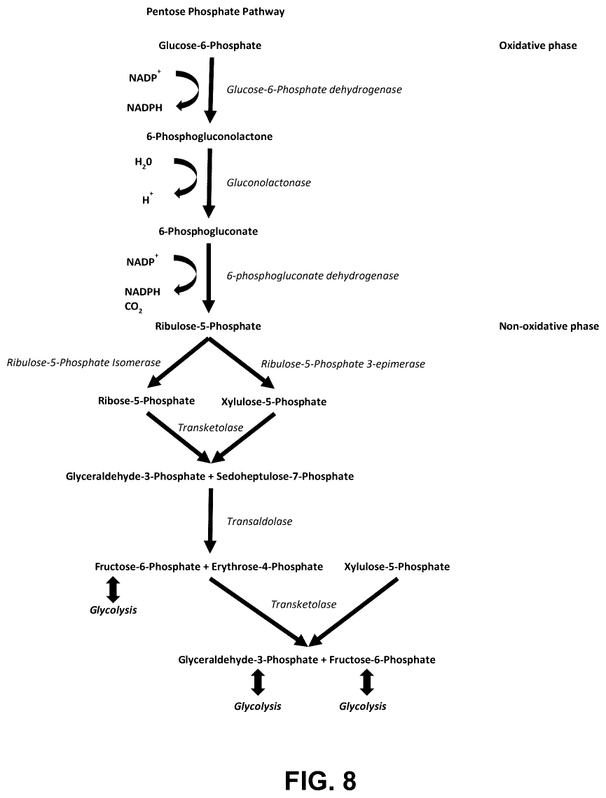

Furthermore, the glycerol production also consumes two molecules of ATP (see FIG. 7). The net ATP production or consumption during glycolysis and glycerol production are summarized in Table 3. Since glycolysis by GDP1 or by NAD⁺ dependent phosphorylating GAPDH is thermodynamically unfavourable, the glycerol production pathway may be favoured over glycolysis. Using the non-phosphorylating GAPDH (GAPN) results in zero net ATP consumption and as such is thermodynamically favorable. Therefore, overexpressing GAPN, may favor the glycolysis pathway over the glycerol production pathway, thereby reducing production of glycerol.

TABLE 3
Estimated Gibbs Energy value of reactions
catalyzed by GAPN and Δzwf1.

| | Net ATP production |
|---|---|
| Reaction pathway | or consumption |
| Glycolysis using GAPN | 0 ATP |
| (EC1.2.1.9) | |
| Glycolysis using GDP1 | +2ATP |
| (EC1.2.1.13) | |
| Glycolysis using NAD⁺ | +2ATP |
| dependent phosphorylating | |
| GAPDH (EC1.2.1.12) | |
| Glycerol production | −2ATP |

Corn fermentation for ethanol production is a metabolically stressful process for Saccharomyces cerevisiae, where fast fermentation kinetics and tolerance to process upsets are important. Blomberg (2000) suggested that a futile cycling of ATP may be an important part of the Saccharomyces cerevisiae stress response pathway. A futile cycle occurs when two metabolic pathways run simultaneously in opposite directions; for example, glycolysis (i.e. conversion of glucose into pyruvate) and gluconeogenesis (i.e. conversion of pyruvate back to glucose) being active at the same time. The overall effect is consumption of ATP. Hence during stress conditions (i.e. fermentation), it may be preferable to avoid higher levels of ATP formation.

### Genetic Modification for Upregulating Conversion of NADH to NAD⁺

In addition to the two genetic modifications presented above, it may be useful to upregulate an additional activity downstream of pyruvate to prevent carbon loss to undesired by-products (i.e. butanediol). In the context of the present disclosure, a recombinant yeast host cell may further have one or more of a third genetic modification for upregulating a third metabolic pathway for converting NADH to NAD⁺. In one embodiment, the third metabolic pathway allows for or is involved in the production of ethanol.

In some embodiments, the third genetic modification comprises introducing one or more third heterologous nucleic acid molecule encoding one or more of a third polypeptide. The third polypeptide can be a heterologous polypeptide or a polypeptide native to the yeast host cell. In other embodiments, the third genetic modification comprises upregulating the third metabolic pathway by increasing native expression of a third polypeptide. In an embodiment, the third genetic modification comprises introducing and expressing at least one of an heterologous nucleic acid molecule encoding at least one of the following third polypeptide: an alcohol/aldehyde dehydrogenase (ADHE), a NAD-linked glutamate dehydrogenase (GDH2) and/or an alcohol dehydrogenase (ADH1, ADH2, ADH3, ADH4, ADH5, ADH6 and/or ADH7). Examples of the third polypeptide are listed in Table 4. Some of these enzymes are involved in pathways that allows for the production of ethanol. For example, bifunctional alcohol/aldehyde dehydrogenase produces ethanol directly from pyruvate.

TABLE 4
Example enzymes sequences that convert NADH to NAD⁺.
For SEQ ID NO: 10 to 18, the amino acid sequence provided
refers to the Saccharomyces cerevisiae sequence. The amino
acid sequence of SEQ ID NO: 66 is from Entamoeba histolytica,
of SEQ ID NO: 68 is from Entamoeba nuttalli and or SEQ ID
NO: 70 is from Entamoeba dispar.

| Gene | Enzyme | SEQ ID NO |
|---|---|---|
| ADHE | Alcohol/aldehyde dehydrogenase | 10 |
| GDH2 | NAD-linked glutamate dehydrogenase | 11 |
| ADH1 | Alcohol dehydrogenase | 12 |
| ADH2 | Alcohol dehydrogenase | 13 |
| ADH3 | Alcohol dehydrogenase | 14 |
| ADH4 | Alcohol dehydrogenase | 15 |
| ADH5 | Alcohol dehydrogenase | 16 |
| ADH6 | Alcohol dehydrogenase | 17 |
| ADH7 | Alcohol dehydrogenase | 18 |
| ADH | Alcohol dehydrogenase | 66 |
| ADH | Alcohol dehydrogenase | 68 |
| ADH | Alcohol dehydrogenase | 70 |

In one embodiment, the third polypeptide comprises a polypeptide having bifunctional alcohol/aldehyde dehydrogenase activity, and has, for example, the amino acid sequence of SEQ ID NO: 10; is a variant of SEQ ID NO: 10, or is a fragment of SEQ ID NO: 10.

In one embodiment, the third polypeptide comprises a polypeptide having NAD-linked glutamate dehydrogenase activity and has, for example, the amino acid sequence of SEQ ID NO: 11; is a variant of SEQ ID NO: 11, or is a fragment of SEQ ID NO: 11.

In one embodiment, the third polypeptide comprises a polypeptide having alcohol dehydrogenase activity that uses NADH as a cofactor. The NADH-dependent alcohol dehydrogenase activity can have, for example, the amino acid sequence of SEQ ID NO: 12 to 18, 66, 68 or 70; is a variant of SEQ ID NO: 12 to 18, 66, 68 or 70, or is a fragment of SEQ ID NO: 12 to 18, 66, 68 or 70.

In another embodiment, the third metabolic pathway allows the production of 1,3-propanediol from the fermentation of glycerol. This can be achieved by expressing a glycerol fermentation pathway. In Clostridium butyricum, the glycerol fermentation pathway is also be referred to as the reuterin pathway. This pathway consists of three genes coding for the following enzymes: a glycerol dehydratase (EC 4.2.1.30), a glycerol dehydratase activating protein, and a 1,3-propanediol dehydrogenase (1.1.1.202). This pathway converts glycerol to 1,3-propanediol, producing one water and one NAD⁺. When coupled with the native yeast glycerol production pathway, 2 NADH are oxidized to 2 NAD⁺, effectively doubling the power of the cell to re-oxidize excess cytosolic NADH resulting from biomass production during anaerobic growth. Ultimately, biomass-linked glycerol production is reduced via increased NADH oxidation through glycerol fermentation to 1,3-propanediol. An additional benefit of this third metabolic pathway is the ability to detoxify reuterin produced by contaminating bacteria in a corn ethanol fermentation. In aqueous solution, 3-hydroxypropionaldehyde (3-HPA) exists in dynamic equilibrium with 3-HPA hydrate, 3-HPA dimer, and acrolein. This system is referred to as reuterin and has been shown to be toxic to many microbes, including yeast. Engineering a yeast host cell to reduce 3-HPA to 1,3-PDO via 1,3-propanediol dehydrogenase activity would prevent accumulation of 3-HPA and therefore reuterin, minimizing the threat of process disruption by contamination by reuterin-producing bacteria.

As such, the one or more third heterologous polypeptide can include a polypeptide having glycerol dehydratase activase activity. The polypeptide having glycerol dehydratase activase activity can be from Clostridium sp., for example from Clostridium butyricum. In an embodiment the polypeptide having glycerol dehydratase activase activity can have the amino acid sequence of SEQ ID NO: 30, be a variant thereof of be a fragment thereof.

The one or more third heterologous polypeptide can also include a polypeptide having glycerol dehydratase activity. The polypeptide having glycerol dehydratase activity can be from Clostridium sp., for example from Clostridium butyricum. In an embodiment the polypeptide having glycerol dehydratase activity can have the amino acid sequence of SEQ ID NO: 32, be a variant thereof of be a fragment thereof.

The one or more third heterologous polypeptide can also include a polypeptide having 1,3-propanediol dehydrogenase activity. The polypeptide having 1,3-propanediol dehydrogenase activity can be from Clostridium sp., for example from Clostridium butyricum. In an embodiment the polypeptide having 1,3-propanediol dehydrogenase activity can have the amino acid sequence of SEQ ID NO: 34, be a variant thereof of be a fragment thereof.

In some embodiment, the third polypeptide is expressed intracellularly and, if necessary, is modified to remove its native signal sequence.

### Genetic Modification for Upregulating Conversion of NADPH to NADP⁺

The present disclosure also provides for recombinant yeast host cells further complemented with upregulation of enzymes that convert NADPH to NADP⁺, allowing for greater regeneration of NADP⁺ for use as cofactor to the glyceraldehyde-3-phosphate dehydrogenase. In the context of the present disclosure, a recombinant yeast host cell may further have one or more of a fourth genetic modification for upregulating a fourth metabolic pathway for converting NADPH to NADP⁺.

In some embodiments, the fourth genetic modification comprises introducing one or more fourth heterologous nucleic acid molecule encoding one or more of a fourth polypeptide. The fourth polypeptide can be a heterologous polypeptide or a polypeptide native to the yeast host cell. In other embodiments, the fourth genetic modification comprises upregulating the fourth metabolic pathway by increasing native expression of a fourth polypeptide. In an embodiment, the fourth genetic modification comprises introducing and expressing a gene encoding at least one of the following fourth polypeptide: mannitol dehydrogenase (DSF1), sorbitol dehydrogenase (SOR1 and/or SOR2) and/or NADPH-dependent alcohol dehydrogenase (ADH6 and/or ADH7). Examples of the fourth polypeptide are listed in Table 5A.

TABLE 5
Example enzymes that convert NADPH to NADP⁺. The amino
acid sequence of SEQ ID NO: 19, 20, 21, 17 and 18 refers
to the Saccharomyces cerevisiae sequence. . The amino acid
sequence of SEQ ID NO: 66 is from Entamoeba histolytica,
of SEQ ID NO: 68 is from Entamoeba nuttalli and or SEQ
ID NO: 70 is from Entamoeba dispar.

| Gene | Enzyme | SEQ ID NO |
|---|---|---|
| DSF1 | Mannitol dehydrogenase | 19 |
| SOR1 | Sorbitol dehydrogenase | 20 |
| SOR2 | Sorbitol dehydrogenase | 21 |
| ADH6 | Alcohol dehydrogenase | 17 |
| ADH7 | Alcohol dehydrogenase | 18 |
| ADH | Alcohol dehydrogenase | 66 |
| ADH | Alcohol dehydrogenase | 68 |
| ADH | Alcohol dehydrogenase | 70 |

In some embodiments, the fourth polypeptide comprises a polypeptide having aldose reductase activity. In one embodiment, the polypeptide having aldose reductase activity is a polypeptide having mannitol dehydrogenase activity and has, for example, the amino acid sequence of SEQ ID NO: 19; is a variant of SEQ ID NO: 19, or is a fragment of SEQ ID NO: 19. In another embodiment, the polypeptide having aldose reductase activity is a polypeptide having sorbitol dehydrogenase activity and has, for example, the amino acid sequence of SEQ ID NO: 20 or 21, is a variant of the amino acid sequence of SEQ ID NO: 20 or 21 or is a fragment of the amino acid sequence of SEQ ID NO: 20 or 21.

In one embodiment, the fourth polypeptide is a polypeptide having alcohol dehydrogenase activity that uses NADPH as a cofactor. The NADPH-dependent alcohol dehydrogenase activity has, for example, the amino acid sequence of SEQ ID NO: 17 or 18; is a variant of SEQ ID NO: 17, 18, 66, 68 or 70, or is a fragment of SEQ ID NO: 17, 18, 66, 68 or 70.

In some embodiment, the fourth polypeptide is expressed intracellularly and, if necessary is modified to as to remove its native signal sequence.

### Genetic Modification for Upregulating Saccharolytic Activity

In some embodiments, the recombinant yeast host cell can include a fifth genetic modification allowing the expression of an heterologous saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

In specific embodiments, the recombinant yeast host cell can bear one or more genetic modifications allowing for the production of an heterologous glucoamylase as the heterologous saccharolytic/amylolytic enzyme. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous polypeptide is derived from a γ-amylase, such as, for example, the glucoamylase of Saccharomycodes fibuligera (e.g., encoded by the glu 0111 gene). The polypeptide having glucoamylase activity can have the amino acid sequence of SEQ ID NO: 28, be a variant thereof or be a fragment thereof. The polypeptide having glucoamylase activity can have the amino acid sequence of SEQ ID NO: 40, be a variant thereof or be a fragment thereof. Additional examples of recombinant yeast host cells bearing such fifth genetic modifications are described in WO 2011/153516 as well as in WO 2017/037614 and herewith incorporated in its entirety.

In specific embodiments, the recombinant yeast host cell can bear one or more genetic modifications allowing for the production of a heterologous trehalase as the heterologous saccharolytic enzyme. As it is known in the art, trehalases are glycoside hydrolases capable of converting trehalose into glucose (E.C. 3.2.1.28). The heterologous trehalase can be derived from any organism. In an embodiment, the heterologous trehalase is from Achlya sp., for example Achlya hypogyna, Ashbya sp., for example Ashbya gossypii, Aspergillus sp., for example from Aspergillus clavatus, Aspergillus flavus, Aspergillus fumigatus, Aspergillus lentulus, Aspergillus ochraceoroseus, from Escovopsis sp., for example from Escovopsis weberi, Fusarium sp., for example from Fusarium oxysporum, Kluyveromyces sp., for example from Kluyveromyces marxianus, Komagataella sp., for example from Komagataella phaffii, Metarhizium sp., for example from Metarhizium anisopliae, om Microsporum sp., for example from Microsporum gypseum, Neosartorya sp., for example from Neosartorya udagawae, Neurospora sp., for example from Neurospora crassa, Ogataea sp., for example from Ogataea parapolymorpha, Rhizoctonia sp., for example from Rhizoctonia solani, Schizopora sp., for example from Schizopora paradoxa, or Thielavia sp., for example from Thielavia terrestris. In some specific embodiments, the heterologous trehalase has the amino acid sequence of SEQ ID NO: 38, is a variant thereof or a fragment thereof.

### Glycerol Production and Transport

The recombinant yeast host cell of the present disclosure can include an optional sixth genetic modification for limiting glycerol production and/or facilitating the transport (and in an embodiment, the export) of glycerol.

Native enzymes that function to produce glycerol include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast host cells bearing such genetic modification(s) leading to the reduction in the production of one or more native enzymes that function to produce glycerol are described in WO 2012/138942. In some embodiments, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). In still another embodiment (in combination or alternative to the genetic modification described above). In yet another embodiment, the recombinant yeast host cell does bear a genetic modification in the GPP/GDP genes and includes its native genes coding for the GPP/GDP polypeptide(s).

Additional enzymes capable of limiting glycerol production include, but are not limited to, the GLT1 polypeptide (having NAD(+)-dependent glutamate synthase activity) and the GLN1 polypeptide (having glutamine synthetase activity). The GLT1 and GLN1 genes form part of the ammonium assimilation pathway. The expression of heterologous GLT1 and GLN1 genes utilise NADH which can result in limiting glycerol production. In the embodiment in which the recombinant yeast host cell express and heterologous GLT1 polypeptide and GLN1 polypeptide, the recombinant yeast host cell can also include an inactivation (e.g., deletion) in the native GDH1 gene. In an example, the GLT1 polypeptide has the amino acid sequence of SEQ ID NO: 43, is a variant of the amino acid sequence of SEQ ID NO: 43 having NAD (+)-dependent glutamate synthase activity or is a fragment of SEQ ID NO: 43 having NAD (+)-dependent glutamate synthase activity. In another example, the GLN1 polypeptide has the amino acid sequence of SEQ ID NO: 45, is a variant of the amino acid sequence of SEQ ID NO: 45 having glutamine synthetase activity or is a fragment of the amino acid sequence of SEQ ID NO: 45 having glutamine synthetase activity.

Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol transport.

The STL1 polypeptide is natively expressed in yeasts and fungi, therefore the heterologous polypeptide functioning to import glycerol can be derived from yeasts and fungi. STL1 genes encoding the STL1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 polypeptide is encoded by Saccharomyces cerevisiae Gene ID: 852149. In an embodiment, the STL1 polypeptide has the amino acid sequence of SEQ ID NO: 26, is a variant of the amino acid sequence of SEQ ID NO: 26 or is a fragment of the amino acid sequence of SEQ ID NO: 26.

### Process for Converting Biomass

The recombinant yeast host cells described herein can be used to improve fermentation yield during fermentation. In some embodiments, the recombinant yeast host cell of the present disclosure maintain their robustness during fermentation in the presence of a stressor such as, for example, lactic acid, formic acid and/or a bacterial contamination (that can be associated, in some embodiments, the an increase in lactic acid during fermentation), an increase in pH, a reduction in aeration, elevated temperatures or combinations. The fermented product can be an alcohol, such as, for example, ethanol, isopropanol, n-propanol, 1-butanol, methanol, acetone and/or 1, 2 propanediol. In an embodiment, the fermented product is ethanol. As shown in the examples, the downregulation of a first pathway involved in NAPD⁺ consumption and the upregulation of a second pathway also involved in NADP⁺ consumption, resulted in increased ethanol yield without increasing glycerol yield compared to fermentation using native yeast host cells without the first and second genetic modification.

The biomass that can be fermented with the recombinant yeast host cells or co-cultures as described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line-rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The process of the present disclosure contacting the recombinant host cells described herein with a biomass so as to allow the conversion of at least a part of the biomass into the fermentation product (e.g., an alcohol such as ethanol). In an embodiment, the biomass or substrate to be hydrolyzed is a lignocellulosic biomass and, in some embodiments, it comprises starch (in a gelatinized or raw form). The process can include, in some embodiments, heating the lignocellulosic biomass prior to fermentation to provide starch in a gelatinized form.

The fermentation process can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the production of ethanol from cellulose can be performed, for example, at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C. In some embodiments, the recombinant microbial host cell can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### Example I—Ethanol and Glycerol Production of ZWF1Δ::GAPN Recombinant Yeast Cells

Fermentation performance of recombinant Saccharomyces cerevisiae strains of Example 1 were evaluated in Verduyn's media with 20 g/L glucose at pH 5.0. Fermentation vessels were sealed, purged with nitrogen, and fitted with one-way valves. Fermentation was carried out with agitation at 35° C. for 24 hours, and samples were analyzed via High Performance Liquid Chromatography (HPLC). As positive control, fcy1 knockout (fcy1Δ) in GAPN background was used. Descriptions of strains included in this fermentation study are described in Table 6. The results of this fermentation study is provided in FIG. 2, and the relative change in ethanol and glycerol production of the strains are summarized in Table 7. Under the experimental conditions used, the highest ethanol (33.1 g/L) and lowest glycerol (2.7 g/L) titers are achieved when GAPN is expressed in combination with zwf1Δ in strain M18913.

TABLE 6
Description of stains evaluated for fermentation performance.

| | | Genes | |
|---|---|---|---|
| | Genes | Overexpressed | |
| Strain | Inactivated | or Introduced | Description |
| M2390 | N.A. | N.A. | Wild type strain |
| M18646 | zwf1 | N.A. | zwf1 deletion |
| M7153 | fcy1 | GAPN | GAPN integrated at |
| | | (SEQ ID NO: 1) | fcy1 locus; zwf1 intact |
| M18913 | zwf1 | GAPN | GAPN integrated at |
| | | (SEQ ID NO: 1) | zwf1 locus; zwf1 deleted |

TABLE 7
Summary of change in ethanol and glycerol production,
relative to wild type strain as reference.

| Strain | Genotype | ΔEthanol | ΔGlycerol |
|---|---|---|---|
| M2390 | WT | 0.0% | 0.0% |
| M18646 | zwf1Δ | −33.5% | −32.9% |
| M7153 | fcy1Δ::GAPN | 0.5% | −26.0% |
| M18913 | zwf1Δ::GAPN | 1.9% | −33.2% |

Strain M7153 expresses the GAPN gene at fcy1Δ, maintaining ZWF1 intact, and in this strain glycerol is reduced by 26%, with a 0.5% increase in ethanol titer. When GAPN is expressed with zwf1 deleted (M18913), glycerol is reduced by 33% accompanied by a 1.9% increase in ethanol titer. A strain deficient in zwf1 (M18646) exhibits methionine auxotrophy and is unable to finish fermentation under these conditions.

### Example II—Characterization of ZWF1Δ::GAPN Recombinant Yeast Cells

Strain propagation. Yeast strains were patched to agar plates containing 1% yeast extract, 2% peptone, 4% glucose and 2% agar (YPD₄₀) from glycerol stocks and were incubated overnight at 35° C. The following day, a loop of cells was inoculated into 30 mL of YPD₄₀, media and grown overnight at 35° C. The overnight cultures were added into the fermentation at a concentration of 0.06 g/L of dry cell weight (DCW).

Verduyn fermentation. Overnight YPD cultures were washed 1× with ddH₂O and inoculated into 25 mL of verduyn media containing 4% glucose, pH 4.2. CO₂ off-gas was measured using a pressure monitoring system (ACAN). Endpoint samples were analyzed for metabolites by HPLC and for DCW.

Mash fermentation. YPD cultures (25 to 50 g) were inoculated into 30-32.5% total solids (TS) corn mash containing lactrol (7 mg/kg) and penicillin (9 mg/kg) in 125 mL bottles fitted with one way valves. Urea was added at a concentration of 0-300 ppm urea depending on substrate used. Exogenous glucoamylase was added at 100%=0.6A GU/gTS and 50-65% for strains expressing a glucoamylase. The strains were incubated at 33° C. for 18 h-48 h, followed by 31° C. for permissive fermentation, 36° C. hold for high temp or 34° C. hold for lactic fermentation, shaking at 150 RPM. 0.38% w/v lactic was added at T=18 h. Samples were collected at 18-68 h depending on the experiment and metabolites were measured using HPLC.

The fermentation characteristics of the Saccharomyces cerevisiae strains described in Table 8 have been determined under permissive and stressful fermentations.

Table 8 Description of stains evaluated for fermentation performance. STL1 refers to the STL1 polypeptide from Saccharomyces cerevisiae having the amino acid sequence of SEQ ID NO: 26. MP1152 refers to a glucoamylase from Saccharomycopsis fibuligera having the amino acid sequence of SEQ ID NO: 28. MP1139 refers to a glycerol dehydratase activase from Clostridium butyricum having the amino acid sequence of SEQ ID NO: 30. MP1140 refers to a glycerol dehydratase from Clostridium butyricum having the amino acid sequence of SEQ ID NO: 32. MP1141 refers to a 1,3-propanediol dehydrogenase from Clostridium butyricum having the amino acid sequence of SEQ ID NO: 34. ADHE refers to the bifunctional alcohol dehydrogenase from Bifidobacterium adolescentis having the amino acid sequence of SEQ ID NO: 36. The trehalase is from Neurospora crassa and has the amino acid sequence of SEQ ID NO: 38. MP743 refers to a glucoamylase from Saccharomycopsis fibuligera having the amino acid sequence of SEQ ID NO: 41. GLT1 is a NAD (+)-dependent glutamate synthase (GOGAT) from Saccharomyces cerevisiae having the amino acid sequence of SEQ ID NO: 43. GLN1 is a glutamine synthetase from Saccharomyces cerevisiae having the amino acid sequence of SEQ ID NO: 45. GAPN Lb is a NADP-dependent glyceraldehyde-3-phosphate dehydrogenase from Lactobacillus delbrueckii having the amino acid sequence of SEQ ID NO: 47. GAPN St is a NADP-dependent glyceraldehyde-3-phosphate dehydrogenase from Streptococcus thermophilus having the amino acid sequence of SEQ ID NO: 49. GAPN Sm is a NADP-dependent glyceraldehyde-3-phosphate dehydrogenase from Streptococcus macacae having the amino acid number of SEQ ID NO: 51. GAPN Sh is a NADP-dependent it glyceraldehyde-3-phosphate dehydrogenase from Streptococcus hyointestinalis having the amino acid sequence of SEQ ID NO: 53. GAPN Su is a NADP-dependent glyceraldehyde-3-phosphate dehydrogenase from Streptococcus urinalis having the amino acid sequence of SEQ ID NO: 55. GAPN Sc is a NADP-dependent glyceraldehyde-3-phosphate dehydrogenase from Streptococcus canis having the amino acid sequence of SEQ ID NO: 57. GAPN Sth is a NADP-dependent glyceraldehyde-3-phosphate dehydrogenase from Streptococcus thoraltensis having the amino acid sequence of SEQ ID NO: 59. GAPN Sd is a NADP-dependent glyceraldehyde-3-phosphate dehydrogenase from Streptococcus dysgalactiae having the amino acid sequence of SEQ ID NO: 61. TSL1 is the large subunit of trehalose 6-phosphate synthase/phosphatase complex from Saccharomyces cerevisiae having the amino acid sequence of SEQ ID NO: 64. GAPN Spy is a NADP-dependent glyceraldehyde-3-phosphate dehydrogenase from Streptococcus pyogenes having the amino acid sequence of SEQ ID NO: 72. GAPN Spi is a NADP-dependent glyceraldehyde-3-phosphate dehydrogenase from Streptococcus ictaluri having the amino acid sequence of SEQ ID NO: 74. GAPN Cp is a NADP-dependent glyceraldehyde-3-phosphate dehydrogenase from Clostridium perfringens having the amino acid sequence of SEQ ID NO: 76. GAPN Cc is a NADP-dependent glyceraldehyde-3-phosphate dehydrogenase from Clostridium chromiireducens having the amino acid sequence of SEQ ID NO: 78. GAPN Cb is a NADP-dependent glyceraldehyde-3-phosphate dehydrogenase from Clostridium botulinum having the amino acid sequence of SEQ ID NO: 80. GAPN Bc is a NADP-dependent glyceraldehyde-3-phosphate dehydrogenase from Bacillus cereus having the amino acid sequence of SEQ ID NO: 82. GAPN Ba is a NADP-dependent glyceraldehyde-3-phosphate dehydrogenase from Bacillus anthracis having the amino acid sequence of SEQ ID NO: 84. GAPN Bt is a NADP-dependent glyceraldehyde-3-phosphate dehydrogenase from Bacillus thuringiensis having the amino acid sequence of SEQ ID NO: 86.

| | | | Genes | | | |
|---|---|---|---|---|---|---|
| | | Genes | Overexpressed | | | |
| Strain | Background | Inactivated | or Introduced | Promoter | Terminator | Description |
| M2390 | N.A. | | | | | Wild type strain |
| M8279 | N.A. | | | | | Wild type strain |
| M19506 | M8279 | | 2 copies of STL1 | adh1p | idp1t | STL1 |
| | | | (SEQ ID NO: 25) | | | overexpressed |
| M18913 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| M19687 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| | | | 4 copies of STL1 | adh1p/stl1p | idp1t/pdc1t | STL1 |
| | | | (SEQ ID NO: 25) | | | overexpressed |
| M22889 | M8279 | zwf1 | 4 copies of GAPN | tpi1p | idp1t/fba1t | GAPN integrated |
| | | | (SEQ ID NO: 1) | | | at the zwf1 locus; |
| | | | | | | zwf1 deleted |
| | | | 2 copies of STL1 | adh1p | idp1t | STL1 |
| | | | (SEQ ID NO: 25) | | | overexpressed |
| M20170 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| | | | 2 copies of STL1 | adh1p | idp1t/pdc1t | STL1 |
| | | | (SEQ ID NO: 25) | | | overexpressed |
| | | | 4 copies of ADHE | pfk1p/tpi1p | hxt2t/fba1t | ADHE |
| | | | (SEQ ID NO: 35) | | | overexpressed |
| M20365 | M2390 | zwf1 | 2 copies of GAPN | tpi1p | fba1t | GAPN integrated |
| | | | (SEQ ID NO: 1) | | | at the zwf1 locus |
| | | | 2 copies of | adh1p | pdc1t | MP1139 |
| | | | MP1139 (SEQ | | | overexpressed |
| | | | ID NO: 29) | | | |
| | | | 2 copies of | eno1p | eno1t | MP1140 |
| | | | MP1140 (SEQ | | | overexpressed |
| | | | ID NO: 31) | | | |
| | | | 2 copies of | pfk1p | hxt2t | MP1141 |
| | | | MP1141 (SEQ | | | overexpressed |
| | | | ID NO: 33) | | | |
| | | | 2 copies of STL1 | adh1p/stl1p | idp1t/pdc1t | STL1 |
| | | | (SEQ ID NO: 25) | | | overexpressed |
| | | | 1 copy of GAPN | tefp | adh3t | GAPN |
| | | | (SEQ ID NO: 1) | | | overexpressed, |
| | | | | | | integrated at |
| | | | | | | additional site |
| | | | 4 copies of | hxt3p/qcr8p | idp1t/pgk1t | MP1152 |
| | | | MP1152 (SEQ ID | | | overexpressed |
| | | | NO: 27) | | | |
| M19994 | M2390 | zwf1 | 2 copies of GAPN | tpi1p | fba1t | GAPN integrated |
| | | | (SEQ ID NO: 1) | | | at the zwf1 locus |
| | | | 4 copies of STL1 | adh1p/stl1p | idp1t/pdc1t | STL1 |
| | | | (SEQ ID NO: 25) | | | overexpressed |
| | | | 4 copies of | hxt3p/qcr8p | idp1t/pgk1t | MP1152 |
| | | | MP1152 (SEQ ID | | | overexpressed |
| | | | NO: 27) | | | |
| M20576 | M8279 | zwf1 | | | | |
| | | | 4 copies of STL1 | tef2p/adh1p | adh3t/pdc1t | STL1 integrated at |
| | | | (SEQ ID NO: 25) | | | ime1 locus |
| | | | 2 copies of | tef2p | adh3t | Trehalase |
| | | | trehalase (SEQ | | | overexpressed |
| | | | ID NO: 37) | | | |
| | | | 2 copies of TSL1 | Mutant tsl1p | tsl1t | TSL1 |
| | | | (SEQ ID NO: 63) | (SEQ ID NO: | | overexpressed |
| | | | | 62) | | |
| | | | 8 copies of | tdh1p/hor7p | pgk1t/idp1t | MP743 |
| | | | MP743 (SEQ ID | | | overexpressed |
| | | | NO: 40) | | | |
| | | | 4 copies of ADHE | pfk1p/tpi1 p | hxt2t/fba1t | ADHE |
| | | | (SEQ ID NO: 35) | | | overexpressed |
| | | | 2 copies of GAPN | tpi1p | fba1t | GAPN integrated |
| | | | (SEQ ID NO: 1) | | | at the zwf1 locus; |
| | | | | | | zwf1 deleted |
| M20922 | M2390 | zwf1 | 2 copies of GAPN | adh1p | fba1t | GAPN integrated |
| | | | (SEQ ID NO: 1) | | | at the zwf1 locus; |
| | | | | | | zwf1 deleted |
| M20923 | M2390 | zwf1 | 2 copies of GAPN | gpd1p | fba1t | GAPN integrated |
| | | | (SEQ ID NO: 1) | | | at the zwf1 locus; |
| | | | | | | zwf1 deleted |
| M20924 | M2390 | zwf1 | 2 copies of GAPN | hxt3p | fba1t | GAPN integrated |
| | | | (SEQ ID NO: 1) | | | at the zwf1 locus; |
| | | | | | | zwf1 deleted |
| M20925 | M2390 | zwf1 | 2 copies of GAPN | qcr8p | fba1t | GAPN integrated |
| | | | (SEQ ID NO: 1) | | | at the zwf1 locus; |
| | | | | | | zwf1 deleted |
| M20926 | M2390 | zwf1 | 2 copies of GAPN | pgi1p | fba1t | GAPN integrated |
| | | | (SEQ ID NO: 1) | | | at the zwf1 locus; |
| | | | | | | zwf1 deleted |
| M20927 | M2390 | zwf1 | 2 copies of GAPN | pfk1p | fba1t | GAPN integrated |
| | | | (SEQ ID NO: 1) | | | at the zwf1 locus; |
| | | | | | | zwf1 deleted |
| M20928 | M2390 | zwf1 | 2 copies of GAPN | fba1p | fba1t | GAPN integrated |
| | | | (SEQ ID NO: 1) | | | at the zwf1 locus; |
| | | | | | | zwf1 deleted |
| M20929 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| M20930 | M2390 | zwf1 | 2 copies of GAPN | tdh2p | fba1t | GAPN integrated |
| | | | (SEQ ID NO: 1) | | | at the zwf1 locus; |
| | | | | | | zwf1 deleted |
| M20931 | M2390 | zwf1 | 2 copies of GAPN | pgk1p | fba1t | GAPN integrated |
| | | | (SEQ ID NO: 1) | | | at the zwf1 locus; |
| | | | | | | zwf1 deleted |
| M20932 | M2390 | zwf1 | GAPN | gpm1p | fba1t | GAPN integrated |
| | | | (SEQ ID NO: 1) | | | at the zwf1 locus; |
| | | | | | | zwf1 deleted |
| M20933 | M2390 | zwf1 | 2 copies of GAPN | eno2p | fba1t | GAPN integrated |
| | | | (SEQ ID NO: 1) | | | at the zwf1 locus; |
| | | | | | | zwf1 deleted |
| M20934 | M2390 | zwf1 | 2 copies of GAPN | cdc19p | fba1t | GAPN integrated |
| | | | (SEQ ID NO: 1) | | | at the zwf1 locus; |
| | | | | | | zwf1 deleted |
| M20935 | M2390 | zwf1 | 2 copies of GAPN | zwf1p | fba1t | GAPN integrated |
| | | | (SEQ ID NO: 1) | | | at the zwf1 locus; |
| | | | | | | zwf1 deleted |
| M20936 | M2390 | zwf1 | 2 copies of GAPN | hor7p | fba1t | GAPN integrated |
| | | | (SEQ ID NO: 1) | | | at the zwf1 locus; |
| | | | | | | zwf1 deleted |
| M23526 | M8279 | zwf1 | 8 copies of GAPN | zwf1p/gpd1p | idp1t/fba1t | GAPN integrated |
| | | | (SEQ ID NO: 1) | | | at the zwf1 locus; |
| | | | | | | zwf1 deleted |
| | | | 2 copies of STL1 | adh1p | idp1t | STL1 |
| | | | (SEQ ID NO: 25) | | | overexpressed |
| | | | 2 copies of GLT1 | hxt3p | idp1t | GLT1 |
| | | | (SEQ ID NO: 42) | | | overexpressed |
| | | | 2 copies of GLN1 | qcr8p | pgk1t | GLN1 |
| | | | (SEQ ID NO: 44) | | | overexpressed |
| M23358 | M8279 | zwf1 | 8 copies of GAPN | zwf1p/gpd1p | idp1t/fba1t | GAPN integrated |
| | | | (SEQ ID NO: 1) | | | at the zwf1 locus; |
| | | | | | | zwf1 deleted |
| | | | 2 copies of STL1 | adh1p | idp1t | STL1 |
| | | | (SEQ ID NO: 25) | | | overexpressed |
| M22882 | M8279 | zwf1 | 4 copies of GAPN | zwf1p/gpd1p | idp1t/fba1t | GAPN integrated |
| | | | (SEQ ID NO: 1) | | | at the zwf1 locus; |
| | | | | | | zwf1 deleted |
| M20032 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| | | | 1 copy of | adh1p | pdc1t | MP1139 |
| | | | MP1139 (SEQ ID | | | overexpressed |
| | | | NO: 29) | | | |
| | | | 1 copy of | eno1p | eno1t | MP1140 |
| | | | MP1140 (SEQ ID | | | overexpressed |
| | | | NO: 31) | | | |
| | | | 1 copy of | pfk1p | hxt2t | MP1141 |
| | | | MP1141 (SEQ ID | | | overexpressed |
| | | | NO: 33) | | | |
| M20296 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| | | | 1 copy of | adh1p | pdc1t | MP1139 |
| | | | MP1139 (SEQ ID | | | overexpressed |
| | | | NO: 29) | | | |
| | | | 1 copy of | eno1p | eno1t | MP1140 |
| | | | MP1140 (SEQ ID | | | overexpressed |
| | | | NO: 31) | | | |
| | | | 1 copy of | pfk1p | hxt2t | MP1141 |
| | | | MP1141 (SEQ ID | | | overexpressed |
| | | | NO: 33) | | | |
| | | | Four copies of | adh1p/stl1p | idp1t/pdc1t | STL1 |
| | | | STL1 (SEQ ID | | | overexpressed |
| | | | NO: 25) | | | |
| M20300 | M2390 | zwf1 | 2 copies of GAPN | tpi1p | fba1t | GAPN integrated |
| | | | (SEQ ID NO: 1) | | | at the zwf1 locus; |
| | | | | | | zwf1 deleted |
| | | | 1 copy of | adh1p | pdc1t | MP1139 |
| | | | MP1139 (SEQ ID | | | overexpressed |
| | | | NO: 29) | | | |
| | | | 1 copy of | eno1p | eno1t | MP1140 |
| | | | MP1140 (SEQ ID | | | overexpressed |
| | | | NO: 31) | | | |
| | | | 1 copy of | pfk1p | hxt2t | MP1141 |
| | | | MP1141 (SEQ ID | | | overexpressed |
| | | | NO: 33) | | | |
| | | | 2 copies of STL1 | adh1p/stl1 p | idp1t/pdc1t | STL1 |
| | | | (SEQ ID NO: 25) | | | overexpressed |
| | | | 1 copy of GAPN | tefp | adh3t | GAPN |
| | | | (SEQ ID NO: 1) | | | overexpressed, |
| | | | | | | integrated at |
| | | | | | | additional site |
| M22883 | M8279 | zwf1 | 8 copies of GAPN | zwf1p/gpd1p | idpt1t/fba1t | GAPN Ld |
| | | | Ld (SEQ ID NO: | | | integrated at the |
| | | | 46) | | | zwf1 locus; zwf1 |
| | | | | | | deleted |
| M22886 | M8279 | zwf1 | 8 copies of GAPN | zwf1p/gpd1p | idpt1t/fba1t | GAPN St |
| | | | St (SEQ ID NO: | | | integrated at the |
| | | | 48) | | | zwf1 locus; zwf1 |
| | | | | | | deleted |
| M22889 | M8279 | zwf1 | 2 copies of STL1 | adh1p | idp1t | STL1 |
| | | | (SEQ ID NO: 25) | | | overexpressed |
| | | | 2 copies of GAPN | tpi1p | fba1t | GAPN integrated |
| | | | (SEQ ID NO: 1) | | | at the zwf1 locus; |
| | | | | | | zwf1 deleted |
| M22890 | M8279 | zwf1 | 2 copies of STL1 | adh1p | idp1t | STL1 |
| | | | (SEQ ID NO: 25) | | | overexpressed |
| | | | 2 copies of GAPN | tpi1p | fba1t | GAPN Ld |
| | | | Ld | | | integrated at the |
| | | | (SEQ ID NO: 46) | | | zwf1 locus; zwf1 |
| | | | | | | deleted |
| M22891 | M8279 | zwf1 | 2 copies of STL1 | adh1p | idp1t | STL1 |
| | | | (SEQ ID NO: 25) | | | overexpressed |
| | | | 2 copies of GAPN | tpi1p | fba1t | GAPN St |
| | | | St | | | integrated at the |
| | | | (SEQ ID NO: 48) | | | zwf1 locus; zwf1 |
| | | | | | | deleted |
| M23688 | M2390 | zwf1 | At least one copy | gpd1p | idp1t/fba1t | GAPN Sm |
| | | | of GAPN Sm | | | integrated at the |
| | | | (SEQ ID NO: 50) | | | zwf1 locus; zwf1 |
| | | | | | | deleted |
| M23692 | M2390 | zwf1 | 2 copies of GAPN | gpd1p | idp1t/fba1t | GAPN Sh |
| | | | Sh (SEQ ID NO: | | | integrated at the |
| | | | 52) | | | zwf1 locus; zwf1 |
| | | | | | | deleted |
| M23693 | M2390 | zwf1 | At least one copy | gpd1p | idp1t/fba1t | GAPN Su |
| | | | of GAPN Su | | | integrated at the |
| | | | (SEQ ID NO: 54) | | | zwf1 locus; zwf1 |
| | | | | | | deleted |
| M23696 | M2390 | zwf1 | 2 copies GAPN | gpd1p | idp1t/fba1t | GAPN Sc |
| | | | Sc (SEQ ID NO: | | | integrated at the |
| | | | 56) | | | zwf1 locus; zwf1 |
| | | | | | | deleted |
| M23700 | M2390 | zwf1 | 2 copies GAPN | gpd 1 p | idp1t/fba1t | GAPN Sth |
| | | | Sth (SEQ ID NO: | | | integrated at the |
| | | | 58) | | | zwf1 locus; zwf1 |
| | | | | | | deleted |
| M23702 | M2390 | zwf1 | 2 copies GAPN | gpd 1 p | idp1t/fba1t | GAPN Sd |
| | | | Sd (SEQ ID NO: | | | integrated at the |
| | | | 60) | | | zwf1 locus; zwf1 |
| | | | | | | deleted |
| M23704 | M2390 | zwf1 | At least one copy | gpd1p | idp1t/fba1t | GAPN Spy |
| | | | of GAPN Spy | | | integrated at the |
| | | | (SEQ ID NO: 71) | | | zwf1 locus; zwf1 |
| | | | | | | deleted |
| M23706 | M2390 | zwf1 | 2 copies of GAPN | gpd1p | idp1t/fba1t | GAPN Spi |
| | | | Spi (SEQ ID NO: | | | integrated at the |
| | | | 73) | | | zwf1 locus; zwf1 |
| | | | | | | deleted |
| M23708 | M2390 | zwf1 | 2 copies of GAPN | gpd1p | idp1t/fba1t | GAPN Cp |
| | | | Cp (SEQ ID NO: | | | integrated at the |
| | | | 75) | | | zwf1 locus; zwf1 |
| | | | | | | deleted |
| M23711 | M2390 | zwf1 | At least one copy | gpd1p | idp1t/fba1t | GAPN Cc |
| | | | of GAPN Cc | | | integrated at the |
| | | | (SEQ ID NO: 77) | | | zwf1 locus; zwf1 |
| | | | | | | deleted |
| M23713 | M2390 | zwf1 | 2 copies of GAPN | gpd1p | idp1t/fba1t | GAPN Cb |
| | | | Cb (SEQ ID NO: | | | integrated at the |
| | | | 79) | | | zwf1 locus; zwf1 |
| | | | | | | deleted |
| M23714 | M2390 | zwf1 | 2 copies of GAPN | gpd1p | idp1t/fba1t | GAPN Bc |
| | | | Bc (SEQ ID NO: | | | integrated at the |
| | | | 81) | | | zwf1 locus; zwf1 |
| | | | | | | deleted |
| M23716 | M2390 | zwf1 | 2 copies of GAPN | gpd1p | idp1t/fba1t | GAPN Ba |
| | | | Ba (SEQ ID NO: | | | integrated at the |
| | | | 83) | | | zwf1 locus; zwf1 |
| | | | | | | deleted |
| M23719 | M2390 | zwf1 | 2 copies of GAPN | gpd1p | idp1t/fba1t | GAPN Bt |
| | | | Bt (SEQ ID NO: | | | integrated at the |
| | | | 85) | | | zwf1 locus; zwf1 |
| | | | | | | deleted |

### Promoter Screen

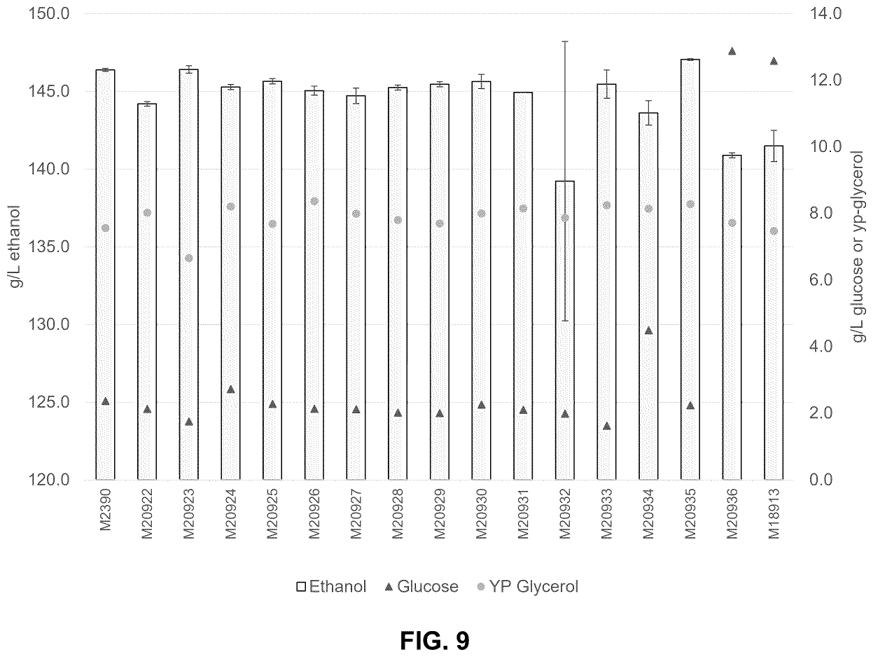

GAPN was expressed with different promoters and the resulting strains were submitted to a fermentation. More specifically, YPD cultures (25 to 50 g) were inoculated into 32.5% total solids (TS) corn mash containing 165 ppm urea, lactrol (7 mg/kg) and penicillin (9 mg/kg) in 125 mL bottles containing one way valves. Exogenous glucoamylase was added at 100%=0.6 AGU/gTS. The strains were incubated at 33° C. for 48 h with shaking (150 RPM). Weight loss was measured at 24 h and 48 h. Endpoint metabolites were measured using HPLC. As shown in FIG. 9, the use of the promoters of the gpd1 (M20923) and zwf1 (strain M20935) genes resulted in a good ethanol yield, while the use of the gpd1 promoter (M20923) lowered glycerol production.

### STL1

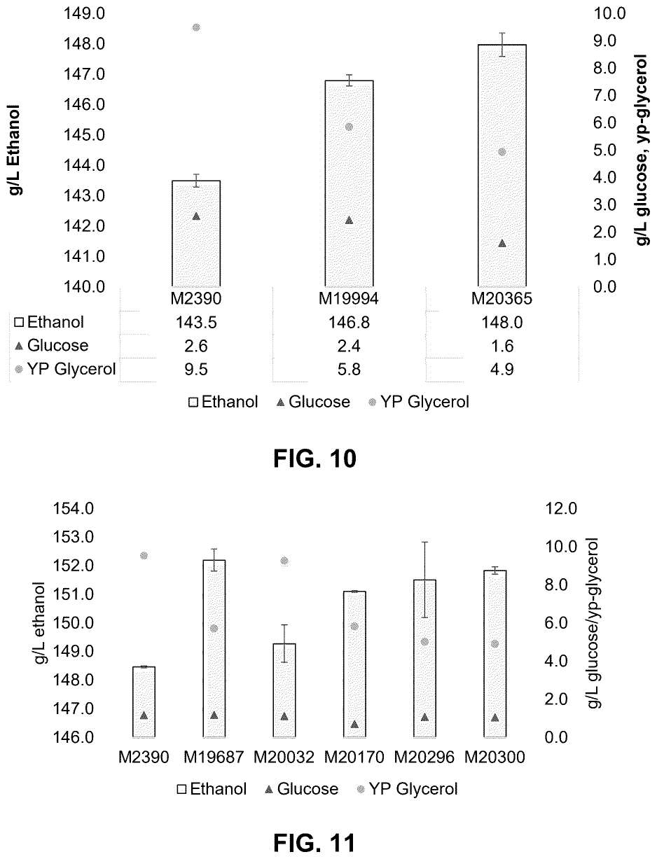

It was then determined if the co-expression of STL1 with GAPN could further increase the fermentation yield in a corn mash fermentation. When STL1 is co-expressed with GAPN, an improvement in the ethanol yield and a reduction in glycerol production is observed (when compared to the parental strain). This is seen in FIG. 10, when STL1 is co-expressed with a glucoamylase (strains M19994 and M20365) as well as in FIG. 11 when STL1 is expressed with GAPN (strain M19687), ADHE (M20170) or in combination with the reuterin complex (strains M20296 and M20300).

### Trehalase

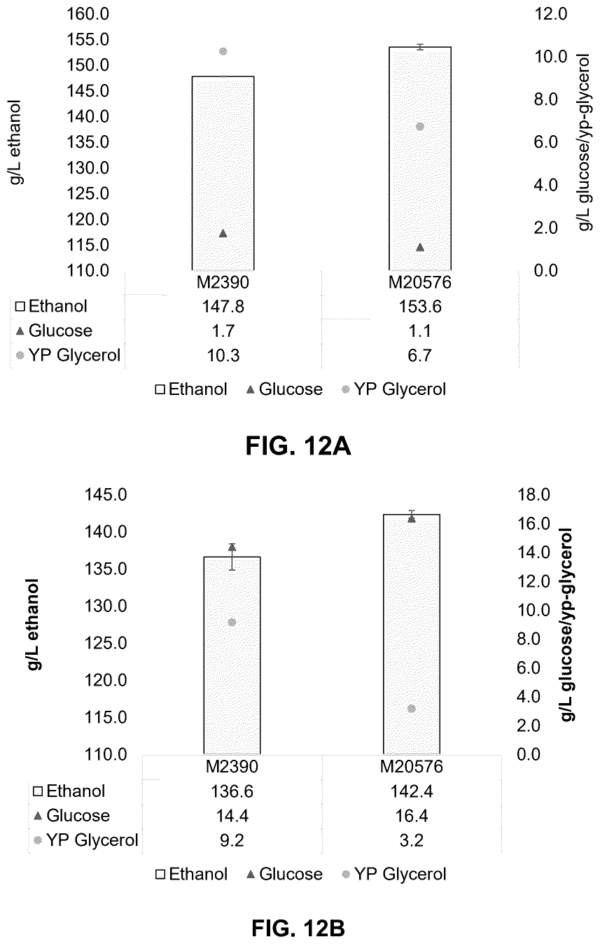

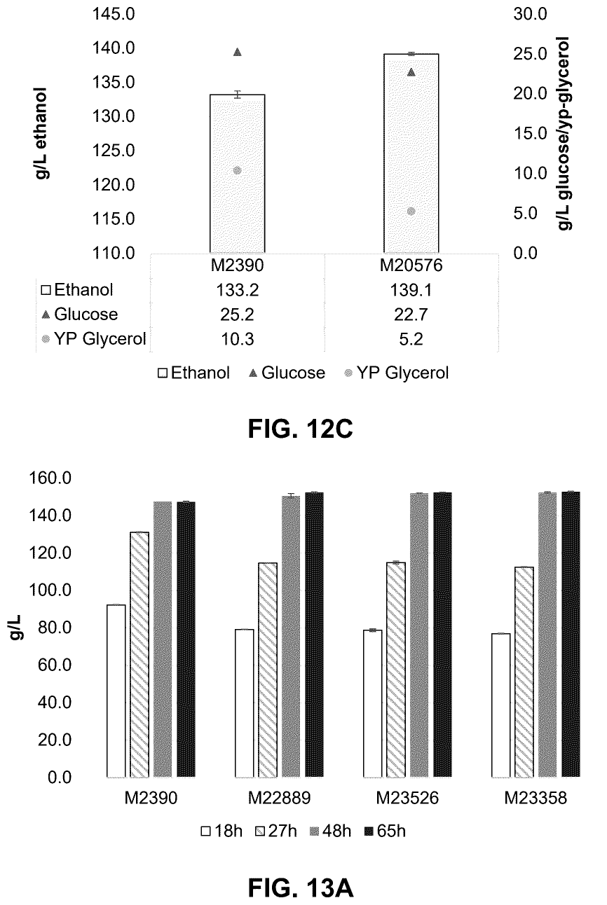

It was also determined if the co-expression of a trehalase with GAPN could increase the fermentation yield in a corn mash fermentation. When a trehalase is co-expressed with GAPN (strain 20576), an increase in ethanol yield and a decrease in glycerol production is observed in permissive (FIG. 12A), lactic acid (FIG. 12B) and high temperature (FIG. 12C) fermentations.

### GLT1/GLN1

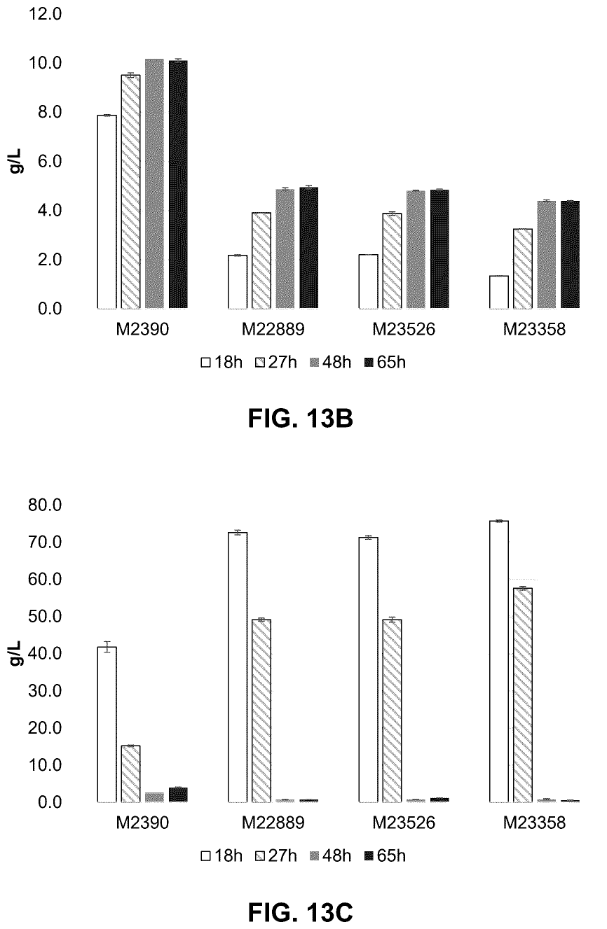

It was determined if the co-expression of GLT1/GLN1 with GAPN could modify the fermentation kinetics of a corn mash fermentation. The co-expression of GLT1/GLN1 with GAPN (strain M23526) increase the ethanol yield (FIG. 13A) while decreasing glycerol production (FIG. 13B) in a corn mash fermentation.

### GAPN Screen

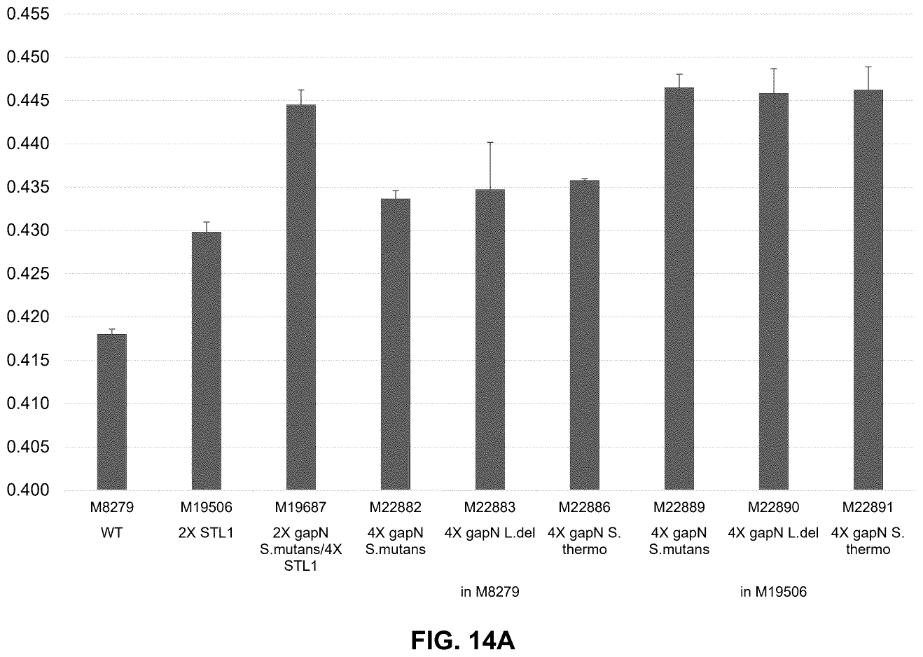

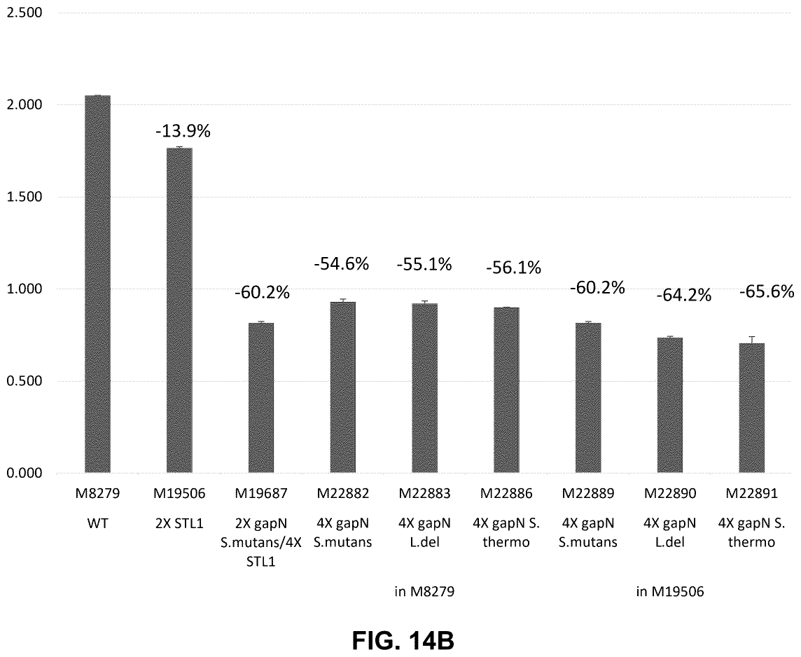

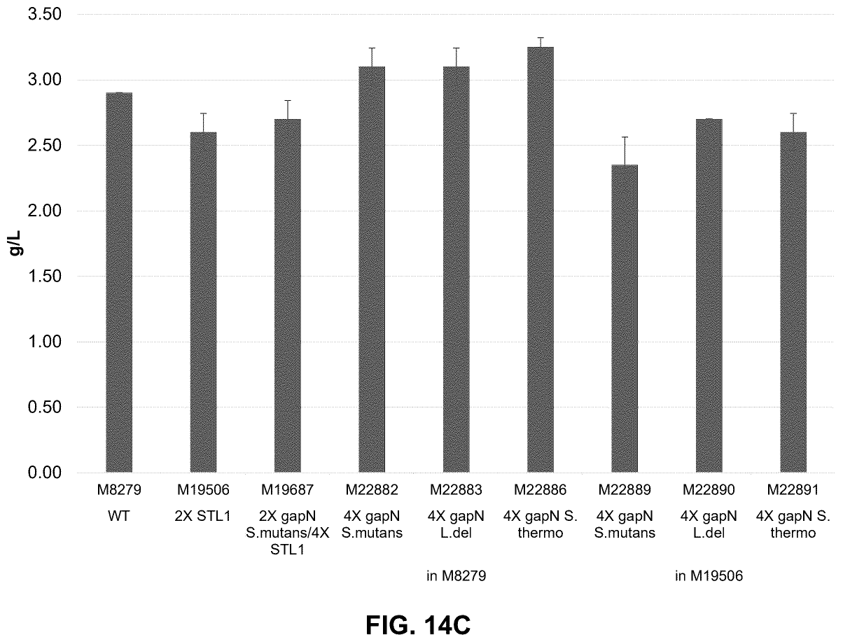

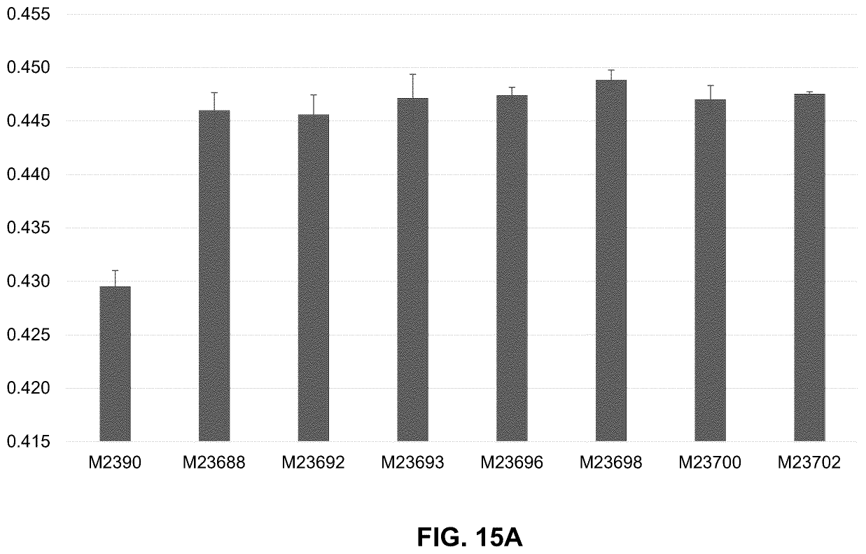

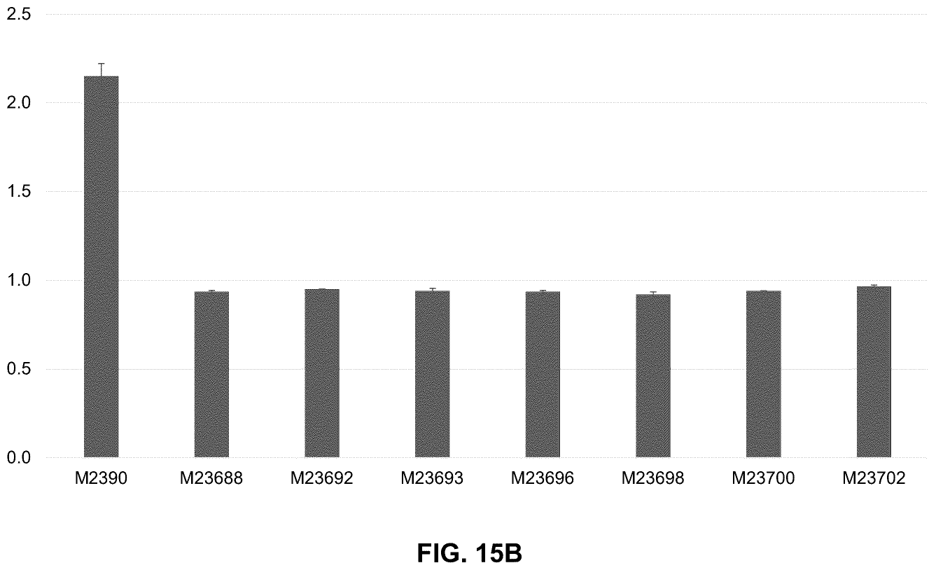

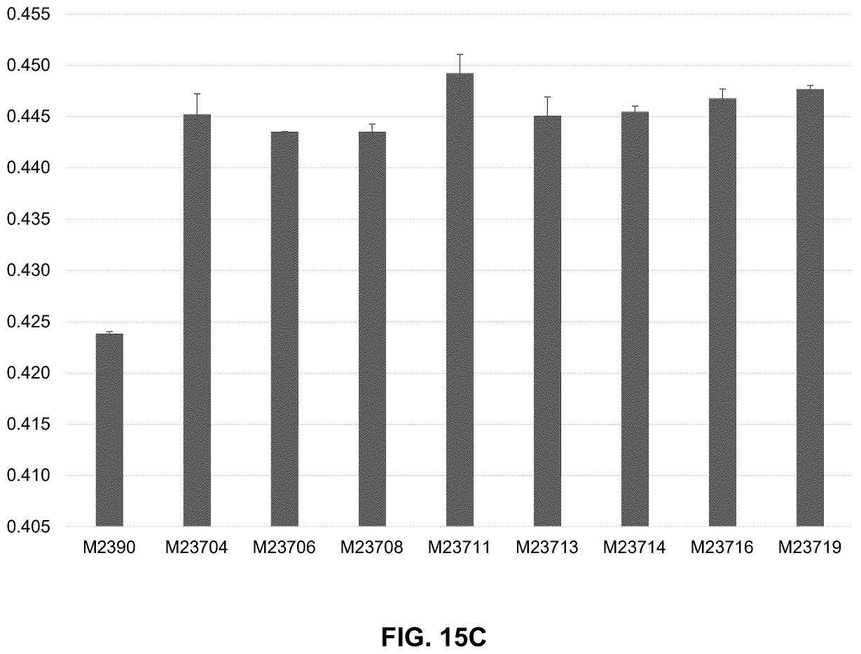

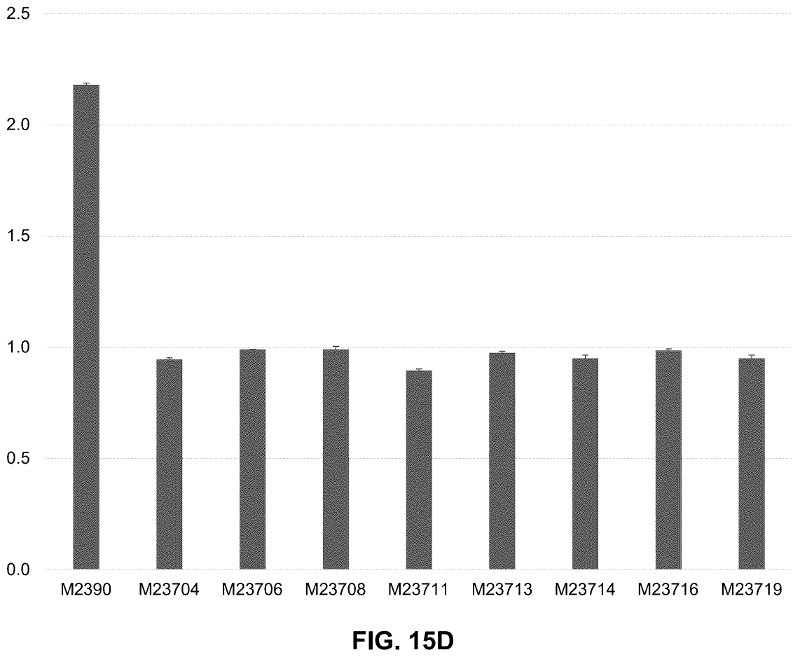

Additional GAPN polypeptides (from Streptococcus thermophilus and Lactobacillus delbrueckii) were screened in different yeast backgrounds. Briefly, yeast strains were patched to agar plates containing 1% yeast extract, 2% peptone, 4% glucose and 2% agar (YPD₄₀) from glycerol stocks and were incubated overnight at 35° C. The following day, a loop of cells was inoculated into 30 mL of YPD₄₀ media and grown overnight at 35° C. The overnight cultures were added into the fermentation at a concentration of 0.06 g/L of dry cell weight (DCW). Overnight YPD cultures were washed 1× with ddH₂O and inoculated into 25 mL of Verduyn media containing 4% glucose, pH 4.2. CO₂ off-gas was measured using a pressure monitoring system (ACAN). Endpoint samples were analyzed for metabolites by HPLC and for DCW. The different GAPN-expressing strains tested all increased ethanol yield (FIGS. 14A, 15A, 15C) and reduced glycerol production (FIGS. 14B, 15B, 15D) when compared to the parental strains in the conditions tested.

### REFERENCES

Blomberg, Anders. Metabolic surprises in Saccharomyces cerevisiae during adaptation to saline conditions: questions, some answers and a model. FEMS Microbiol Lett. 2000 Jan. 1; 182(1):1-8.
Verho et al., Engineering Redox Cofactor Regeneration for Improved Pentose Fermentation in Saccharomyces cerevisiae. Applied and Environmental Microbiology, October 2003, p. 5892-5897.
Zhang et al., Improving the ethanol yield by reducing glycerol formation using cofactor regulation in Saccharomyces cerevisiae. Biotechnol Lett (2011) 33:1375-1380.
Zhang et al., Engineering of the glycerol decomposition pathway and cofactor regulation in an industrial yeast improves ethanol production. J Ind Microbiol Biotechnol (2013) 40:1153-1160.
U.S. Pat. No. 8,956,851
CA2506195
CN100363490

1. A recombinant yeast host cell having:
i) one or more of a first genetic modification for facilitating the transport of glycerol in the recombinant yeast host cell; and
ii) one or more of a second genetic modification for upregulating a second metabolic pathway, wherein the one or more second genetic modification allows the expression of a glyceraldehyde-3-phosphate dehydrogenase lacking phosphorylating activity, wherein the glyceraldehyde-3-phosphate dehydrogenase is of enzyme commission (EC) 1.2.1.9 or 1.2.1.90;
wherein the second metabolic pathway allows the conversion of NADP⁺ to NADPH.

2. The recombinant yeast host cell of claim 1, wherein the one or more first genetic modification comprises inactivation of at least one first native gene.

3.-17. (canceled)

18. The recombinant yeast host cell of claim 1, wherein the one or more second genetic modification comprises introduction of one or more second heterologous nucleic acid molecule encoding the glyceraldehyde-3-phosphate dehydrogenase.

19.-21. (canceled)

22. The recombinant yeast host cell of claim 18, wherein the one or more second heterologous nucleic acid molecule is under the control of a heterologous promoter.

23. The recombinant yeast host cell of claim 22, wherein the heterologous promoter comprises the promoter of an ADH1, GPD1, HXT3, QCR8, PGI1, PFK1, FBA1, TDH2, PGK1, GPM1, ENO2, CDCl9, ZWF1, HOR7, TPI1, TEF2, CWP2, SSA1, ENO1, HXK1, HXT7, GPD2, FZF1, SSU1, TDH1, PAU5, TDH3, PDC1, DAN1, CYC1 and/or QCR8T gene.

24. The recombinant yeast host cell of claim 1, wherein the glyceraldehyde-3-phosphate dehydrogenase is of EC 1.2.1.90.

25. The recombinant yeast host cell of claim 24, wherein the glyceraldehyde-3-phosphate dehydrogenase is GAPN.

26. The recombinant yeast host cell of claim 25, wherein GAPN has:
(a) the amino acid sequence of SEQ ID NO: 2, 47, 49, 51, 53, 55, 57, 59, 61, 72, 74, 76, 78, 80, 82, 84 or 86;
(b) is a variant of SEQ ID NO: 2, 47, 49, 51, 53, 55, 57, 59, 61, 72, 74, 76, 78, 80, 82, 84 or 86 having glyceraldehyde-3-phosphate dehydrogenase activity; or
(c) is a fragment of SEQ ID NO: 2, 47, 49, 51, 53, 55, 57, 59, 61, 72, 74, 76, 78, 80, 82, 84 or 86 having glyceraldehyde-3-phosphate dehydrogenase activity.

27. The recombinant yeast host cell of claim 1, wherein the glyceraldehyde-3-phosphate dehydrogenase is of EC 1.2.1.9.

28.-52. (canceled)

53. The recombinant yeast host cell of claim 1, further
comprising a genetic modification for expressing a heterologous polypeptide having saccharolytic activity.

54.-58. (canceled)

59. The recombinant yeast host cell of claim 1, wherein the one or more first genetic modification comprises introduction of a first heterologous nucleic acid molecule encoding a heterologous polypeptide for facilitating the transport of glycerol in the recombinant yeast host cell.

60. The recombinant yeast host cell of claim 59, wherein the sixth heterologous polypeptide for facilitating the transport of glycerol comprises a STL1 polypeptide having glycerol proton symporter activity.

61. The recombinant yeast host cell of claim 60, wherein the STL1 polypeptide has the amino acid sequence of SEQ ID NO: 26, is a variant of the amino acid sequence of SEQ ID NO: 26 having glycerol proton symporter activity, or is a fragment of the amino acid sequence of SEQ ID NO: 26 having glycerol proton symporter activity.

62.-64. (canceled)

65. The recombinant yeast host cell of claim 1 being from the genus Saccharomyces.

66. The recombinant yeast host cell claim 65 being from the species Saccharomyces cerevisiae.

67. A process for converting a biomass into a fermentation product, the process comprises contacting the biomass with the recombinant yeast host cell defined in claim 1 to allow the conversion of at least a part of the biomass into the fermentation product.

68.-72. (canceled)

73. The recombinant yeast host cell of claim 18, wherein the one or more second heterologous nucleic acid molecule is under the control of a native promoter.

74. The recombinant yeast host cell of claim 73, wherein the native promoter comprises a promoter from a ZWF1, GND1, GND2, ALD6, IDP1, IDP2 and/or IDP3 gene.

75. The recombinant yeast host cell of claim 2, wherein the at least one first native gene comprises a FPS1 polypeptide having glycerol exporter activity.


SEQUENCE LISTING (88 sequences)

SEQ ID NO: 1 - DNA, 1428 bp, Streptococcus mutans
  source 1..1428: mol_type = other DNA
atgacaaaacaatataaaaattatgtcaatggcgagtggaagctttcagaaaatgaaattaaaatctacgaaccggccagtggagctgaattgggttcagttccagcaatgagtactgaagaagtagattatgtttatgcttcagccaagaaagctcaaccagcttggcgatcactttcatacatagaacgtgctgcctaccttcataaggtagcagatattttgatgcgtgataaagaaaaaataggtgctgttctttccaaagaggttgctaaaggttataaatcagcagtcagcgaagttgttcgtactgcagaaatcattaattatgcagctgaagaaggccttcgtatggaaggtgaagtccttgaaggcggcagttttgaagcagccagcaagaaaaaaattgccgttgttcgtcgtgaaccagtaggtcttgtattagctatttcaccatttaactaccctgttaacttggcaggttcgaaaattgcaccggctcttattgcgggaaatgttattgcttttaaaccaccgacgcaaggatcaatctcagggctcttacttgctgaagcatttgctgaagctggacttcctgcaggtgtctttaataccattacaggtcgtggttctgaaattggagactatattgtagaacatcaagccgttaactttatcaatttcactggttcaacaggaattggggaacgtattggcaaaatggctggtatgcgtccgattatgcttgaactcggtggaaaagattcagccatcgttcttgaagatgcagaccttgaattgactgctaaaaatattattgcaggtgcttttggttattcaggtcaacgctgtacagcagttaaacgtgttcttgtgatggaaagtgttgctgatgaactggtcgaaaaaatccgtgaaaaagttcttgcattaacaattggtaatccagaagacgatgcagatattacaccgttgattgatacaaaatcagctgattatgtagaaggtcttattaatgatgccaatgataaaggagccgctgcccttactgaaatcaaacgtgaaggtaatcttatctgtccaatcctctttgataaggtaacgacagatatgcgtcttgcttgggaagaaccatttggtcctgttcttccgatcattcgtgtgacatctgtagaagaagccattgaaatttctaacaaatcggaatatggacttcaggcttctatctttacaaatgatttcccacgcgcttttggtattgctgagcagcttgaagttggtacagttcatatcaataataagacacagcgcggtacggacaacttcccattcttaggggctaaaaaatcaggtgcaggtattcaaggggtaaaatattctattgaagctatgacaactgttaaatccgtcgtatttgatatcaaataa

SEQ ID NO: 2 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFLNFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTIDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 3 - protein, 504 aa, Saccharomyces cerevisiae
MSEGPVKFEKNTVISVFGASGDLAKKKTFPALFGLFREGYLDPSTKIFGYARSKLSMEDLKSRVLPHLKKPHGEADDSKVEQFFKMVSYISGNYDTDEGFDELRTQIEKFEKSANVDVPHRLFYLALPPSVFLTVAKQIKSRVYAENGITRVIVEKPFGHDLASARELQKNLGPLFKEEELYRIDHYLGKELVKNLLVLRFGNQFLNASWNRDNIQSVQISFKERFGTEGRGGYFDSIGIIRDVMQNHLLQIMTLLTMERPVSFDPESIRDEKVKVLKAVAPIDTDDVLLGQYGKSEDGSKPAYVDDDTVDKDSKCVTFAAMTFNIENERWEGVPIMMRAGKALNESKVEIRLQYKAVASGVFKDIPNNELVIRVQPDAAVYLKFNAKTPGLSNATQVTDLNLTYASRYQDFWIPEAYEVLIRDALLGDHSNFVRDDELDISWGIFTPLLKHIERPDGPTPEIYPYGSRGPKGLKEYMQKHKYVMPEKHPYAWPVTKPEDTKDN

SEQ ID NO: 4 - protein, 489 aa, Saccharomyces cerevisiae
MSADFGLIGLAVMGQNLILNAADHGFTVCAYNRTQSKVDHFLANEAKGKSIIGATSIEDFISKLKRPRKVMLLVKAGAPVDALINQIVPLLEKGDIIIDGGNSHFPDSNRRYEELKKKGILFVGSGVSGGEEGARYGPSLMPGGSEEAWPHIKNIFQSISAKSDGEPCCEWVGPAGAGHYVKMVHNGIEYGDMQLICEAYDIMKRLGGFTDKEISDVFAKWNNGVLDSFLVEITRDILKFDDVDGKPLVEKIMDTAGQKGTGKWTAINALDLGMPVTLIGEAVFARCLSALKNERIRASKVLPGPEVPKDAVKDREQFVDDLEQALYASKIISYAQGFMLIREAAATYGWKLNNPAIALMWRGGCIIRSVFLGQITKAYREEPDLENLLFNKFFADAVTKAQSGWRKSIALATTYGIPTPAFSTALSFYDGYRSERLPANLLQAQRDYFGAHTFRVLPECASDNLPVDKDIHINWTGHGGNVSSSTYQA

SEQ ID NO: 5 - protein, 492 aa, Saccharomyces cerevisiae
MSKAVGDLGLVGLAVMGQNLILNAADHGFTVVAYNRTQSKVDRFLANEAKGKSIIGATSIEDLVAKLKKPRKIMLLIKAGAPVDTLIKELVPHLDKGDIIIDGGNSHFPDTNRRYEELTKQGILFVGSGVSGGEDGARFGPSLMPGGSAEAWPHIKNIFQSIAAKSNGEPCCEWVGPAGSGHYVKMVHNGIEYGDMQLICEAYDIMKRIGRFTDKEISEVFDKWNTGVLDSFLIEITRDILKFDDVDGKPLVEKIMDTAGQKGTGKWTAINALDLGMPVTLIGEAVFARCLSAIKDERKRASKLLAGPTVPKDAIHDREQFVYDLEQALYASKIISYAQGFMLIREAARSYGWKLNNPAIALMWRGGCIIRSVFLAEITKAYRDDPDLENLLFNEFFASAVTKAQSGWRRTIALAATYGIPTPAFSTALAFYDGYRSERLPANLLQAQRDYFGAHTFRILPECASAHLPVDKDIHINWTGHGGNISSSTYQA

SEQ ID NO: 6 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 7 - protein, 428 aa, Saccharomyces cerevisiae
MSMLSRRLFSTSRLAAFSKIKVKQPVVELDGDEMTRIIWDKIKKKLILPYLDVDLKYYDLSVESRDATSDKITQDAAEAIKKYGVGIKCATITPDEARVKEFNLHKMWKSPNGTIRNILGGTVFREPIVIPRIPRLVPRWEKPIIIGRHAHGDQYKATDTLIPGPGSLELVYKPSDPTTAQPQTLKVYDYKGSGVAMAMYNTDESIEGFAHSSFKLAIDKKLNLFLSTKNTILKKYDGRFKDIFQEVYEAQYKSKFEQLGIHYEHRLIDDMVAQMIKSKGGFIMALKNYDGDVQSDIVAQGFGSLGLMTSILVTPDGKTFESEAAHGTVTRHYRKYQKGEETSTNSIASIFAWSRGLLKRGELDNTPALCKFANILESATLNTVQQDGIMTKDLALACGNNERSAYVTTEEFLDAVEKRLQKEIKSIE

SEQ ID NO: 8 - protein, 412 aa, Saccharomyces cerevisiae
MTKIKVANPIVEMDGDEQTRIIWHLIRDKLVLPYLDVDLKYYDLSVEYRDQTNDQVTVDSATATLKYGVAVKCATITPDEARVEEFHLKKMWKSPNGTIRNILGGTVFREPIIIPRIPRLVPQWEKPIIIGRHAFGDQYKATDVIVPEEGELRLVYKSKSGTHDVDLKVFDYPEHGGVAMMMYNTTDSIEGFAKASFELAIERKLPLYSTTKNTILKKYDGKFKDVFEAMYARSYKEKFESLGIWYEHRLIDDMVAQMLKSKGGYIIAMKNYDGDVESDIVAQGFGSLGLMTSVLITPDGKTFESEAAHGTVTRHFRQHQQGKETSTNSIASIFAWTRGIIQRGKLDNTPDVVKFGQILESATVNTVQEDGIMTKDLALILGKSERSAYVTTEEFIDAVESRLKKEFEAAAL

SEQ ID NO: 9 - protein, 420 aa, Saccharomyces cerevisiae
MSKIKVVHPIVEMDGDEQTRVIWKLIKEKLILPYLDVDLKYYDLSIQERDRTNDQVTKDSSYATLKYGVAVKCATITPDEARMKEFNLKEMWKSPNGTIRNILGGTVFREPIIIPKIPRLVPHWEKPIIIGRHAFGDQYRATDIKIKKAGKLRLQFSSDDGKENIDLKVYEFPKSGGIAMAMFNTNDSIKGFAKASFELALKRKLPLFFTTKNTILKNYDNQFKQIFDNLFDKEYKEKFQALKITYEHRLIDDMVAQMLKSKGGFIIAMKNYDGDVQSDIVAQGFGSLGLMTSILITPDGKTFESEAAHGTVTRHFRKHQRGEETSTNSIASIFAWTRAIIQRGKLDNTDDVIKFGNLLEKATLDTVQVGGKMTKDLALMLGKTNRSSYVTTEEFIDEVAKRLQNMMLSSNEDKKGMCKL

SEQ ID NO: 10 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 11 - protein, 1092 aa, Saccharomyces cerevisiae
MLFDNKNRGALNSLNTPDIASLSISSMSDYHVFDFPGKDLQREEVIDLLDQQGFIPDDLIEQEVDWFYNSLGIDDLFFSRESPQLISNIIHSLYASKLDFFAKSKFNGIQPRLFSIKNKIITNDNHAIFMESNTGVSISDSQQKNFKFASDAVGNDTLEHGKDTIKKNRIEMDDSCPPYELDSEIDDLFLDNKSQKNCRLVSFWAPESELKLTFVYESVYPNDDPAGVDISSQDLLKGDIESISDKTMYKVSSNENKKLYGLLLKLVKEREGPVIKTTRSVENKDEIRLLVAYKRFTTKRYYSALNSLFHYYKLKPSKFYLESFNVKDDDIIIFSVYLNENQQLEDVLLHDVEAALKQVEREASLLYAIPNNSFHEVYQRRQFSPKEAIYAHIGAIFINHFVNRLGSDYQNLLSQITIKRNDTTLLEIVENLKRKLRNETLTQQTIINIMSKHYTIISKLYKNFAQIHYYHNSTKDMEKTLSFQRLEKVEPFKNDQEFEAYLNKFIPNDSPDLLILKTLNIFNKSILKTNFFITRKVAISFRLDPSLVMTKFEYPETPYGIFFVVGNTFKGFHIRFRDIARGGIRIVCSRNQDIYDLNSKNVIDENYQLASTQQRKNKDIPEGGSKGVILLNPGLVEHDQTFVAFSQYVDAMIDILINDPLKENYVNLLPKEEILFFGPDEGTAGFVDWATNHARVRNCPWWKSFLTGKSPSLGGIPHDEYGMTSLGVRAYVNKIYETLNLTNSTVYKFQTGGPDGDLGSNEILLSSPNECYLAILDGSGVLCDPKGLDKDELCRLAHERKMISDFDTSKLSNNGFFVSVDAMDIMLPNGTIVANGTTFRNTFHTQIFKFVDHVDIFVPCGGRPNSITLNNLHYFVDEKTGKCKIPYIVEGANLFITQPAKNALEEHGCILFKDASANKGGVTSSSMEVLASLALNDNDFVHKFIGDVSGERSALYKSYVVEVQSRIQKNAELEFGQLWNLNQLNGTHISEISNQLSFTINKLNDDLVASQELWLNDLKLRNYLLLDKIIPKILIDVAGPQSVLENIPESYLKVLLSSYLSSTFVYQNGIDVNIGKFLEFIGGLKREAEASA

SEQ ID NO: 12 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 13 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 14 - protein, 375 aa, Saccharomyces cerevisiae [sequence omitted]

SEQ ID NO: 15 - protein, 382 aa, Saccharomyces cerevisiae
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 16 - protein, 351 aa, Saccharomyces cerevisiae
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

SEQ ID NO: 17 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 18 - protein, 361 aa, Saccharomyces cerevisiae
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 19 - protein, 502 aa, Saccharomyces cerevisiae
MTKSDETTATSLNAKTLKSFESTLPIPTYPREGVKQGIVHLGVGAFHRSHLAVFMHRLMQEHHLKDWSICGVGLMKADALMRDAMKAQDCLYTLVERGIKDTNAYIVGSITAYMYAPDDPRAVIEKMANPDTHIVSLTVTENGYYHSEATNSLMTDAPEIINDLNHPEKPDTLYGYLYEALLLRYKRGLTPFTIMSCDNMPQNGVTVKTMLVAFAKLKKDEKFAAWIEDKVTSPNSMVDRVTPRCTDKERKYVADTWGIKDQCPVVAEPFIQWVLEDNFSDGRPPWELVGVQVVKDVDSYELMKLRLLNGGHSAMGYLGYLAGYTYIHEVVNDPTINKYIRVLMREEVIPLLPKVPGVDFEEYTASVLERFSNPAIQDTVARICLMGSGKMPKYVLPSIYEQLRKPDGKYKLLAVCVAGWFRYLTGVDMNGKPFEIEDPMAPTLKAAAVKGGKDPHELLNIEVLFSPEIRDNKEFVAQLTHSLETVYDKGPIAAIKEILDQV

SEQ ID NO: 20 - protein, 357 aa, Saccharomyces cerevisiae
MSQNSNPAVVLEKVGDIAIEQRPIPTIKDPHYVKLAIKATGICGSDIHYYRSGGIGKYILKAPMVLGHESSGQVVEVGDAVTRVKVGDRVAIEPGVPSRYSDETKEGRYNLCPHMAFAATPPIDGTLVKYYLSPEDFLVKLPEGVSYEEGACVEPLSVGVHSNKLAGVRFGTKVVVFGAGPVGLLTGAVARAFGATDVIFVDVFDNKLQRAKDFGATNTFNSSQFSTDKAQDLADGVQKLLGGNHADVVFECSGADVCIDAAVKTTKVGGTMVQVGMGKNYTNFPIAEVSGKEMKLIGCFRYSFGDYRDAVNLVATGKVNVKPLITHKFKFEDAAKAYDYNIAHGGEVVKTIIFGPE

SEQ ID NO: 21 - protein, 357 aa, Saccharomyces cerevisiae
MSQNSNPAVVLEKVGDIAIEQRPIPTIKDPHYVKLAIKATGICGSDIHYYRSGGIGKYILKAPMVLGHESSGQVVEVGDAVTRVKVGDRVAIEPGVPSRYSDETKEGSYNLCPHMAFAATPPIDGTLVKYYLSPEDFLVKLPEGVSYEEGACVEPLSVGVHSNKLAGVRFGTKVVVFGAGPVGLLTGAVARAFGATDVIFVDVFDNKLQRAKDFGATNTFNSSQFSTDKAQDLADGVQKLLGGNHADVVFECSGADVCIDAAVKTTKVGGTMVQVGMGKNYTNFPIAEVSGKEMKLIGCFRYSFGDYRDAVNLVATGKVNVKPLITHKFKFEDAAKAYDYNIAHGGEVVKTIIFGPE

SEQ ID NO: 22 - protein, 332 aa, Saccharomyces cerevisiae
MIRIAINGFGRIGRLVLRLALQRKDIEVVAVNDPFISNDYAAYMVKYDSTHGRYKGTVSHDDKHIIIDGVKIATYQERDPANLPWGSLKIDVAVDSTGVFKELDTAQKHIDAGAKKVVITAPSSSAPMFVVGVNHTKYTPDKKIVSNASCTTNCLAPLAKVINDAFGIEEGLMTTVHSMTATQKTVDGPSHKDWRGGRTASGNIIPSSTGAAKAVGKVLPELQGKLTGMAFRVPTVDVSVVDLTVKLEKEATYDQIKKAVKAAAEGPMKGVLGYTEDAVVSSDFLGDTHASIFDASAGIQLSPKFVKLISWYDNEYGYSARVVDLIEYVAKA

SEQ ID NO: 23 - protein, 332 aa, Saccharomyces cerevisiae
MVRVAINGFGRIGRLVMRIALQRKNVEVVALNDPFISNDYSAYMFKYDSTHGRYAGEVSHDDKHIIVDGHKIATFQERDPANLPWASLNIDIAIDSTGVFKELDTAQKHIDAGAKKVVITAPSSTAPMFVMGVNEEKYTSDLKIVSNASCTTNCLAPLAKVINDAFGIEEGLMTTVHSMTATQKTVDGPSHKDWRGGRTASGNIIPSSTGAAKAVGKVLPELQGKLTGMAFRVPTVDVSVVDLTVKLNKETTYDEIKKVVKAAAEGKLKGVLGYTEDAVVSSDFLGDSNSSIFDAAAGIQLSPKFVKLVSWYDNEYGYSTRVVDLVEHVAKA

SEQ ID NO: 24 - protein, 332 aa, Saccharomyces cerevisiae
MVRVAINGFGRIGRLVMRIALSRPNVEVVALNDPFITNDYAAYMFKYDSTHGRYAGEVSHDDKHIIVDGKKIATYQERDPANLPWGSSNVDIAIDSTGVFKELDTAQKHIDAGAKKVVITAPSSTAPMFVMGVNEEKYTSDLKIVSNASCTTNCLAPLAKVINDAFGIEEGLMTTVHSLTATQKTVDGPSHKDWRGGRTASGNIIPSSTGAAKAVGKVLPELQGKLTGMAFRVPTVDVSVVDLTVKLNKETTYDEIKKVVKAAAEGKLKGVLGYTEDAVVSSDFLGDSHSSIFDASAGIQLSPKFVKLVSWYDNEYGYSTRVVDLVEHVAKA

SEQ ID NO: 25 - DNA, 1710 bp, Saccharomyces cerevisiae
  source 1..1710: mol_type = other DNA
atgaaggatttaaaattatcgaatttcaaaggcaaatttataagcagaaccagtcactggggacttacgggtaagaagttgcggtatttcatcactatcgcatctatgacgggcttctccctgtttggatacgaccaagggttgatggcaagtctaattactggtaaacagttcaactatgaatttccagcaaccaaagaaaatggcgatcatgacagacacgcaactgtagtgcagggcgctacaacctcctgttatgaattaggttgtttcgcaggttctctattcgttatgttctgcggtgaaagaattggtagaaaaccattaatcctgatgggttccgtaataaccatcattggtgccgttatttctacatgcgcatttcgtggttactgggcattaggccagtttatcatcggaagagtcgtcactggtgttggaacagggttgaatacatctactattcccgtttggcaatcagaaatgtcaaaagctgaaaatagagggttgctggtcaatttagaaggttccacaattgcttttggtactatgattgcttattggattgattttgggttgtcttataccaacagttctgttcagtggagattccccgtgtcaatgcaaatcgtttttgctctcttcctgcttgctttcatgattaaactacctgaatcgccacgttggctgatttctcaaagtcgaacagaagaagctcgctacttggtaggaacactagacgacgcggatccaaatgatgaggaagttataacagaagttgctatgcttcacgatgctgttaacaggaccaaacacgagaaacattcactgtcaagtttgttctccagaggcaggtcccaaaatcttcagagggctttgattgcagcttcaacgcaatttttccagcaatttactggttgtaacgctgccatatactactctactgtattattcaacaaaacaattaaattagactatagattatcaatgatcataggtggggtcttcgcaacaatctacgccttatctactattggttcattttttctaattgaaaagctaggtagacgtaagctgtttttattaggtgccacaggtcaagcagtttcattcacaattacatttgcatgcttggtcaaagaaaataaagaaaacgcaagaggtgctgccgtcggcttatttttgttcattacattctttggtttgtctttgctatcattaccatggatatacccaccagaaattgcatcaatgaaagttcgtgcatcaacaaacgctttctccacatgtactaattggttgtgtaactttgcggttgtcatgttcaccccaatatttattggacagtccggttggggttgctacttattttttgctgttatgaattatttatacattccagttatcttctttttctaccctgaaaccgccggaagaagtttggaggaaatcgacatcatctttgctaaagcatacgaggatggcactcaaccatggagagttgctaaccatttgcccaagttatccctacaagaagtcgaagatcatgccaatgcattgggctcttatgacgacgaaatggaaaaagaggactttggtgaagatagagtagaagacacctataaccaaattaacggcgataattcgtctagttcttcaaacatcaaaaatgaagatacagtgaacgataaagcaaattttgagggttga

SEQ ID NO: 26 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 27 - DNA, 1527 bp, Saccharomycopsis fibuligera
  source 1..1527: mol_type = other DNA
atgaggttcccatctattttcaccgctgttttgtttgctgcttcttctgctttggctaacaccggtcatttccaagcttattctggttataccgttaacagagctaacttcacccaatggattcatgaacaaccagctgtttcttggtactacttgttgcaaaacatcgattacccagaaggtcaattcaaagctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 28 - protein, 508 aa, Saccharomycopsis fibuligera
MRFPSIFTAVLFAASSALANTGHFQAYSGYTVNRANFTQWIHEQPAVSWYYLLQNIDYPEGQFKAAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 29 - DNA, 915 bp, Clostridium butyricum
  source 1..915: mol_type = other DNA
atgtccaaagaaatcaagggcgtcttgttcaatatccaaaagttctcattgcatgacggtccaggtattagaactatcgtttttttcaagggctgctccatgtcttgtttgtggtgttctaatccagaaagccaagagattaagccacaagtcatgttcaacaagaacttgtgtactaagtgtggtaggtgtaagtctgaatgtaaatccgctgctatcgatatgaactccgagtacagaattgataagtctaagtgtaccgaatgcaccaagtgtgttgataactgtttgtctggtgctttggttactgaaggtagaaactactctgttgaggacgtcatcaaagaattgaagaaggattccgttcagtacagacgttctaatggtggtattactttatccggtggtgaagttttgttgcaaccagattttgccgttgagttgttgaaagaatgcaaatcttatggttggcataccgctattgaaaccgctatgtatgttaactccgaatccgttaagaaggtcatcccttatattgatttggccatgatcgacatcaagtccatgaatgacgaaatccacaaaaagttcaccggtgtctctaacgaaatcatcttgcaaaacatcaagctgtccgatgaattggccaaagagattattatcagaatcccagtcatcgaaggtttcaatgctgacttgcaatctattggtgctattgcccaattctctaagtctttgactaacttgaagaggatcgacttgttgccataccataattacggtgaaaacaagtaccaagccatcggtagagaatactcattgaaagagttgaagtctccctcaaaggacaagatggaaagattgaaagccttggttgaaatcatgggtattccatgtacaattggtgccgaatga

SEQ ID NO: 30 - protein, 304 aa, Clostridium butyricum
MSKEIKGVLFNIQKFSLHDGPGIRTIVFFKGCSMSCLWCSNPESQEIKPQVMFNKNLCTKCGRCKSECKSAAIDMNSEYRIDKSKCTECTKCVDNCLSGALVTEGRNYSVEDVIKELKKDSVQYRRSNGGITLSGGEVLLQPDFAVELLKECKSYGWHTAIETAMYVNSESVKKVIPYIDLAMIDIKSMNDEIHKKFTGVSNEIILQNIKLSDELAKEIIIRIPVIEGFNADLQSIGAIAQFSKSLTNLKRIDLLPYHNYGENKYQAIGREYSLKELKSPSKDKMERLKALVEIMGIPCTIGAE

SEQ ID NO: 31 - DNA, 2364 bp, Clostridium butyricum
  source 1..2364: mol_type = other DNA
atgatctccaagggtttctctactcaaaccgaaagaatcaacattttgaaggcccaaattttgaacgctaagccatgtgttgaatccgaaagagctattttgatcaccgaatctttcaagcaaactgaaggtcaaccagcaattttgagaagggctttagccttgaaacacatcttggaaaacattccaatcaccatcagggaccaagaattgatagttggttctttgacaaaagagcccagatcctctcaagtttttccagaattttctaacaagtggttgcaggacgaattggacagattgaacaaaagaactggtgatgccttccagatctccgaagaatctaaagaaaagctgaaggacgttttcgaatactggaatggtaagactacttctgaattggctacttcctacatgactgaagaaactagagaagccgttaactgtgatgttttcactgttggtaactactactacaacggtgttggtcatgtttctgttgattacggtaaggttttgagagttggtttcaacggtattatcaacgaagccaaagaacagttggaaaagtccagatctattgacccagacttcatcaagaaagagaagttcttgaactccgtcatcatttcttgtgaagctgctattacctacgttaacagatacgctaaaaaggccaaagaaattgccgataacacttccgatgctaagagaaaagcagaattgaacgaaattgccaagatctgctctaaggtttctggtgaaggtgccaaatctttttatgaagcttgtcagttgttctggttcatccatgccattatcaacatcgaatctaacggtcattctatctctccagctagatttgaccagtatatgtacccttactacgagaacgataagaacatcactgataagttcgcccaagagttgattgattgcatttggatcaagctgaacgacatcaacaaagttagggacgaaatttctactaagcactttggtggttacccaatgtaccaaaatttgatcgttggtggtcaaaactccgaaggtaaagatgctacaaacaaggtttcttacatggctttggaagctgctgttcatgttaagttgccacaaccatctttgtccgttagaatttggaacaagactccagacgaattcttgttaagagctgctgaattgacaagagaaggtttgggtttgccagcttactacaatgatgaagttattatcccagccttggtgtctagaggtttgactttagaagatgctagagactacggtatcattggttgtgttgaaccacaaaaaccaggtaagactgaaggttggcatgattccgcttttttcaatttggctagaatcgtcgaactgaccattaactctggttttgacaagaacaagcaaatcggtccaaagactcagaacttcgaagaaatgaagtccttcgacgaattcatgaaggcttacaaagctcagatggaatacttcgttaagcacatgtgttgtgccgataactgcattgatattgctcatgctgaaagagcaccattgccatttttatcctctatggttgataactgtatcggcaaaggtaaatccttgcaagatggtggtgctgagtacaatttttctggtcctcaaggtgttggtgttgctaatattggtgattctttagttgccgtcaaaaagatcgtctttgacgaaaacaagatcaccccatccgaattgaagaaaaccttgaacaacgacttcaagaacagcgaagaaattcaagccttgttgaagaatgctccaaagttcggtaacgatatcgatgaagttgataatttggccagagaaggtgctttggtttactgtagagaagttaacaagtacactaacccaagaggtggtaattttcaaccaggtctatatccctcctccatcaatgtttatttcggttctttaactggtgctaccccagatggtagaaaatctggtcaaccattggctgatggtgtttctccatcaagaggttgtgatgttagtggtccaactgctgcttgtaattctgtttctaagctggatcatttcattgcttccaacggcaccttgtttaatcaaaagtttcatccatctgccttgaagggtgataatggtttgatgaacttgtcctccttgatcagatcttacttcgatcaaaagggtttccacgtccaattcaacgtcattgataagaagattttgttggctgcccaaaagaaccctgaaaagtatcaagatttgattgtcagagttgctggttactccgctcagtttatttcattggataagtccatccagaacgacattattgctagaaccgaacacgttatgtaa

SEQ ID NO: 32 - protein, 787 aa, Clostridium butyricum
MISKGFSTQTERINILKAQILNAKPCVESERAILITESFKQTEGQPAILRRALALKHILENIPITIRDQELIVGSLTKEPRSSQVFPEFSNKWLQDELDRLNKRTGDAFQISEESKEKLKDVFEYWNGKTTSELATSYMTEETREAVNCDVFTVGNYYYNGVGHVSVDYGKVLRVGFNGIINEAKEQLEKSRSIDPDFIKKEKFLNSVIISCEAAITYVNRYAKKAKEIADNTSDAKRKAELNEIAKICSKVSGEGAKSFYEACQLFWFIHAIINIESNGHSISPARFDQYMYPYYENDKNITDKFAQELIDCIWIKLNDINKVRDEISTKHFGGYPMYQNLIVGGQNSEGKDATNKVSYMALEAAVHVKLPQPSLSVRIWNKTPDEFLLRAAELTREGLGLPAYYNDEVIIPALVSRGLTLEDARDYGIIGCVEPQKPGKTEGWHDSAFFNLARIVELTINSGFDKNKQIGPKTQNFEEMKSFDEFMKAYKAQMEYFVKHMCCADNCIDIAHAERAPLPFLSSMVDNCIGKGKSLQDGGAEYNFSGPQGVGVANIGDSLVAVKKIVFDENKITPSELKKTLNNDFKNSEEIQALLKNAPKFGNDIDEVDNLAREGALVYCREVNKYTNPRGGNFQPGLYPSSINVYFGSLTGATPDGRKSGQPLADGVSPSRGCDVSGPTAACNSVSKLDHFIASNGTLFNQKFHPSALKGDNGLMNLSSLIRSYFDQKGFHVQFNVIDKKILLAAQKNPEKYQDLIVRVAGYSAQFISLDKSIQNDIIARTEHVM

SEQ ID NO: 33 - DNA, 1158 bp, Clostridium butyricum
  source 1..1158: mol_type = other DNA
atgaggatgtacgattatttggtcccatccgttaatttcatgggtgctaattctgtttccgttgttggtgaaagatgcaagattttaggtggtaagaaggctttgatcgttaccgataagttcttgaaggatatggaaggtggtgctgttgaattgactgtgaagtatttgaaagaagccggtttggacgttgtttactatgatggtgttgaacctaatccaaaggatgtcaacgttatcgaaggcctgaagattttcaaagaagaaaactgcgatatgatcgtcactgttggtggtggttcttctcatgattgtggtaaaggtattggtattgctgctactcatgaaggtgacttgtatgattatgctggtatcgaaactttggtcaatccattgccaccaatagttgctgttaacactactgctggtactgcttctgaattgacaagacattgtgttttgaccaacaccaagaagaagatcaagttcgttatcgtgtcttggagaaacttgccattggtttctattaacgacccaatgttgatggttaagaaaccagcaggtttgactgctgctacaggtatggatgctttgactcatgctattgaagcttacgtttctaaggatgctaacccagttactgatgcttctgctattcaagccattaagttgatctcccaaaacttgagacaagctgttgctttgggtgaaaatttggaagctcgtgagaatatggcttacgcttctttgttggctggtatggcttttaacaatgctaacttgggttacgttcatgctatggctcatcaattaggtggtctatatgatatggcacatggtgttgctaatgctatgttgttgccacatgttgaaaggtacaacatgatctctaacccaaagaagttcgctgatattgctgagtttatgggtgagaacatttccggtttgtctgttatggaagctgctgaaaaagctattaacgccatgttcagattgtccgaagatgttggtattcccaagtccttgaaagaaatgggtgttaagcaagaggatttcgaacatatggctgaattggctttgttagatggtaacgctttctccaatccaagaaagggtaatgccaaggatatcattaacattttcaaggccgcttactaa

SEQ ID NO: 34 - protein, 385 aa, Clostridium butyricum
MRMYDYLVPSVNFMGANSVSVVGERCKILGGKKALIVTDKFLKDMEGGAVELTVKYLKEAGLDVVYYDGVEPNPKDVNVIEGLKIFKEENCDMIVTVGGGSSHDCGKGIGIAATHEGDLYDYAGIETLVNPLPPIVAVNTTAGTASELTRHCVLTNTKKKIKFVIVSWRNLPLVSINDPMLMVKKPAGLTAATGMDALTHAIEAYVSKDANPVTDASAIQAIKLISQNLRQAVALGENLEARENMAYASLLAGMAFNNANLGYVHAMAHQLGGLYDMAHGVANAMLLPHVERYNMISNPKKFADIAEFMGENISGLSVMEAAEKAINAMFRLSEDVGIPKSLKEMGVKQEDFEHMAELALLDGNAFSNPRKGNAKDIINIFKAAY

SEQ ID NO: 35 - DNA, 2733 bp, Bifidobacterium adolescentis
  source 1..2733: mol_type = other DNA
atggccgacgccaagaagaaagaagaacctactaagccaaccccagaagaaaaattggctgctgctgaagctgaagttgatgctttggttaagaaaggtttgaaggccttggacgaattcgaaaaattggatcaaaagcaagtcgatcacatcgttgctaaagcttcagttgctgctttgaacaaacatttggttttggctaagatggccgttgaagaaactcatagaggtttggttgaagataaggccaccaagaatattttcgcttgtgaacatgtcaccaactatttggctggtcaaaagaccgttggtatcattagagaagatgatgttttgggtatcgacgaaattgctgaaccagttggtgttgttgctggtgttactccagttactaatccaacttctaccgctattttcaagtccttgattgccttgaaaaccagatgcccaattatctttggttttcatccaggtgctcaaaactgttctgttgctgctgctaaaatcgttagagatgctgctattgctgctggtgctccagaaaactgtattcaatggattgaacacccatccattgaagctactggtgctttgatgaagcacgatggtgttgctactattttggctactggtggtccaggtatggttaaggctgcttattcttctggtaaaccagctttgggtgttggtgctggtaatgctccagcttatgttgataagaacgttgatgttgttagagctgccaacgatttgattttgtctaagcacttcgactacggtatgatttgtgctactgaacaagctattatcgccgataaggatatctatgctccattggtcaaagaattgaagagaagaaaggcctacttcgttaatgctgacgaaaaagctaagttggaacagtatatgttcggttgtaccgcttactctggtcaaactccaaagttgaattctgttgttccaggtaagtccccacagtatattgctaaagctgccggtttcgaaattccagaagatgctacaattttggccgctgaatgtaaagaagtcggagaaaacgaaccattgaccatggaaaaattggcaccagttcaagctgttttgaagtccgataacaaagaacaagccttcgaaatgtgcgaagccatgttgaaacatggtgctggtcatactgctgctattcatacaaacgatagagacttggtcagagaatacggtcaaagaatgcatgcctgcagaattatttggaactctccatcttctttgggtggtgttggtgatatctacaatgctattgctccatctttgactttgggttgtggttcttatggtggtaattctgtttccggtaatgttcaagccgtcaacttgattaacatcaagagaatcgctagaagaaacaacaacatgcaatggttcaagattccagctaagacttactttgaacctaacgccatcaagtacctaagagatatgtacggtatcgaaaaggctgttatcgtttgcgataaggtcatggaacaattgggtatcgttgataagatcatcgatcaattgagagccagatctaacagagttaccttcagaatcatcgattacgttgaaccagaaccatctgttgaaacagttgaaaggggtgctgctatgatgagagaagaatttgaacctgataccattattgctgttggtggtggttctccaatggatgcttctaagattatgtggttgttgtacgaacacccagaaatttcattctccgatgtcagagaaaagttcttcgacattagaaagagagcctttaagattccaccattgggtaaaaaggccaagttggtatgtattccaacctcttcaggtactggttctgaagttactccattcgctgttattaccgatcataagactggttacaagtacccaattaccgattatgctttgactccatctgttgctatcgttgatccagttttggctagaactcaacctagaaaattggcttctgatgctggttttgatgctttgacacatgcttttgaagcctacgtttctgtttacgctaacgatttcactgatggtatggctttacatgctgctaaattggtttgggataacttggctgaatccgttaatggtgaaccaggtgaagaaaaaactagagcccaagaaaagatgcataacgctgctactatggctggtatggcatttggttctgcttttttgggtatgtgtcatggtatggctcatacaattggtgctttgtgtcatgttgctcatggtagaactaactccattttgttgccatacgtcatcagatacaacggttctgttcctgaagaacctacatcttggccaaagtacaacaagtatattgccccagaaagataccaagaaatcgctaagaacttgggtgttaatccaggtaaaactcctgaagaaggtgttgaaaatttggctaaggctgtcgaagattacagagataacaagttgggtatgaacaagtccttccaagaatgtggtgttgacgaagattactactggtccattatcgatcaaattggtatgagagcctacgaagatcaatgtgctccagctaatccaagaattccacaaatcgaagatatgaaggatattgctattgccgcttactacggtgtttctcaagctgaaggtcataagttgagagttcaaagacaaggtgaagctgctacagaagaagcttctgaaagagcttaa

SEQ ID NO: 36 - protein, 910 aa, Bifidobacterium adolescentis; identical to SEQ ID NO: 10

SEQ ID NO: 37 - DNA, 2079 bp, Neurospora crassa
  source 1..2079: mol_type = other DNA
atggtcagtagatttttgggtgctactgttccattggctgctgctattttgccaggtgctagagcattatatgttaacggttctgttactgctccatgcgattctccaatctactgttatggtgaattattgcaccaagtcgaattggctagaccattctctgattctaagacctttgttgatatgccaaccatcaagccagttgatgaagttttggaagctttctctaagttgaccttgccattgtctaacaactccgaattgcatgaattcttgtctacttactttggtccagctggtggtgaattggaagctgttccaactgatcaattgcatgtttctccaactttcttggacaacgtttccgatgatgttatcaagcaattcgttgactccgttattaacatttggccagatttgaccagaaagtatgttggtgccggtgaattgtgtactggttgtgctgattctttcatcccagttaacagaacttttgttgttgctggtggtagattcagagaaccatattactgggattctttctggatcttggaaggtttgttgagaactggtggtgctttcactgaaatctccaagaacattatcgaaaactttttggacttggtcgaacaaatcggttttgttccaaatggtgctagattgtactacttggatagatctcaaccaccattattgacccaaatggttagaatctacgttgaacataccaacgacacctccattttggaaagagctgttcctgttttgaagaaagaatgggaatggtggactaccaacagaactgttgaagttactgctgatggtaagacctactcattgcaaagataccacgttgacaacaatcaacctagaccagaatcttacagagaagattacattaccgccaacaacaactcttactatgctacctctggtatcatctacccagaaactactccattgaacgatactcaaaaggctttgttgtacgctaatttggcttctggtgctgaatctggttgggattattcttctagatggttgaagaatccaggtgatgctgctagagatgtttactttccattgagatccttgaacgtcttggaaatcgttccagttgatttgaactccatcttgtaccaaaacgaagttaccatcggtaagttcttggctcaacaaggttctaaagatgaagctgaagaatgggctaaaaaggccgaagaaagatctgaagctatgtacaagttgatgtggaactctactttgtggtcctacttcgattacaacttgacctcttcttctcaaaacatctacgttccagctgatccacaagtttttccatttgaacaaccatctggtactccagaaggttaccaagttttgttctccgtcaatcaaatgtttccattctggactggtgctgctccagatcaattgaaaggtaatccattagctgttaagttggccttcgaaagaatcaagaacttgttggataacaaggccggtggtattccagctactaattttgttactggtcaacaatgggatgaacctaatgtttggccaccattgatgcatgttttgatggatggtttattgaacactccagctacctttggtgaagatgatccagcttatcaagaaactcaaaccttggctttgagattggctcaaagatacgttgattctactttctgtacttggtatgctactggtggttctacttctgaaactccaaaattgcaaggtttgggttctgatttgaagggtatcatgttcgaaaagtactccgataactctacaaacgttgctggttcaggtggtgaatatgaagttgttgaaggttttggttggaccaacggtgttttgatttgggctgctgataagtttggtgacaagttgaaaagaccagattgcggtgatattactccagctcaagttggtaaaagagccgatattactatggaaaagagagccgttgaattggacgtttttgatgctaagttcaccaagaagtttgccagaaagggtaaattggaaaagttgaaggccaagttcaaaagaagagctgccatttag

SEQ ID NO: 38 - protein, 692 aa, Neurospora crassa
MVSRFLGATVPLAAAILPGARALYVNGSVTAPCDSPIYCYGELLHQVELARPFSDSKTFVDMPTIKPVDEVLEAFSKLTLPLSNNSELHEFLSTYFGPAGGELEAVPTDQLHVSPTFLDNVSDDVIKQFVDSVINIWPDLTRKYVGAGELCTGCADSFIPVNRTFVVAGGRFREPYYWDSFWILEGLLRTGGAFTEISKNIIENFLDLVEQIGFVPNGARLYYLDRSQPPLLTQMVRIYVEHTNDTSILERAVPVLKKEWEWWTTNRTVEVTADGKTYSLQRYHVDNNQPRPESYREDYITANNNSYYATSGIIYPETTPLNDTQKALLYANLASGAESGWDYSSRWLKNPGDAARDVYFPLRSLNVLEIVPVDLNSILYQNEVTIGKFLAQQGSKDEAEEWAKKAEERSEAMYKLMWNSTLWSYFDYNLTSSSQNIYVPADPQVFPFEQPSGTPEGYQVLFSVNQMFPFWTGAAPDQLKGNPLAVKLAFERIKNLLDNKAGGIPATNFVTGQQWDEPNVWPPLMHVLMDGLLNTPATFGEDDPAYQETQTLALRLAQRYVDSTFCTWYATGGSTSETPKLQGLGSDLKGIMFEKYSDNSTNVAGSGGEYEVVEGFGWTNGVLIWAADKFGDKLKRPDCGDITPAQVGKRADITMEKRAVELDVFDAKFTKKFARKGKLEKLKAKFKRRAAI

SEQ ID NO: 39 - DNA, 1548 bp, Saccharomycopsis fibuligera
  source 1..1548: mol_type = other DNA
atgatcagattgaccgttttcttgaccgctgtttttgctgctgttgcttcttgtgttccagttgaattggataagagaaacaccggtcatttccaagcttattctggttataccgttaacagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 40 - DNA, 1548 bp, Saccharomycopsis fibuligera
  source 1..1548: mol_type = other DNA
atgatcagattgaccgttttcttgaccgctgtttttgctgctgttgcttcttgtgttccagttgaattggataagagaaacaccggtcatttccaagcttattctggttataccgttgctagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 41 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 42 - DNA, 6438 bp, Saccharomyces cerevisiae
  source 1..6438: mol_type = other DNA
atgccagtgttgaaatcagacaatttcgatccattggaagaagcttacgaaggtgggacaattcaaaactataacgatgaacaccatcttcataaatcttgggcaaatgtgattccggacaaacgaggactttacgaccctgattatgaacatgacgcttgtggtgtcggtttcgtagcaaataagcatggtgaacagtctcacaagattgttactgacgctagatatcttttagtgaatatgacacatcgtggtgccgtctcatctgatggtaacggtgacggtgccggtattctgctaggtattcctcacgaatttatgaaaagagaattcaagttagatcttgatctagacatacctgagatgggcaaatacgccgtaggtaacgtcttcttcaagaagaacgaaaaaaataacaagaaaaatttaattaagtgtcagaagattttcgaggatttagctgcatccttcaacttatccgtattaggttggagaaacgtccccgtagattctactattttaggagacgttgcattatctcgtgaacctactattctacagccattattggttccattgtatgatgaaaaacaaccggagtttaatgaaactaaatttagaactcaattgtatcttttaaggaaggaggcctctcttcaaataggactggaaaactggttctatgtttgttccctaaacaataccaccattgtttacaagggtcaattgacgccagctcaagtgtataactactatcccgacttgactaatgcgcatttcaaatcccacatggcgttggtccattcaagattttccactaatactttcccctcttgggatagagctcagcctttacgttggctagctcataatggtgaaattaacaccttaagaggtaacaagaattggatgcgctccagagaaggtgtgatgaattcagcaactttcaaagatgagttagacaaactatacccaattatcgaagaaggtggttctgattcagctgcattggataacgttttagaactattgactattaatggcacattatctctacctgaagctgtaatgatgatggttcctgaagcgtatcataaggatatggattctgacctaaaagcatggtacgactgggctgcatgtctgatggaaccttgggatggtccagctttgttaactttcactgatggacgttactgtggtgctatattggatagaaatggtttaagaccttgtcgttattacatcactagtgatgacagagttatctgtgcttcagaggtaggtgtcattcctatcgaaaattcattggttgttcaaaaaggtaaactgaagccaggtgatttattcctagtggatactcaattgggtgaaatggtcgatactaaaaagttaaaatctcaaatctcaaaaagacaagattttaagtcttggttatccaaagtcatcaagttagacgacttgttatcaaaaaccgctaatttagttcctaaagaatttatatcacaggattcattgtctttgaaagttcaaagtgacccacgtctattggccaatggttataccttcgaacaagtcacatttctgttaactccaatggctttaacaggtaaagaagctttaggttcgatgggtaacgatgcgccactggcttgtttaaatgaaaatcctgtcttactttatgattatttcagacaattgtttgctcaagtgaccaatcctccaattgacccaattcgtgaagcaaatgttatgtcgttagaatgttatgtcggacctcaaggcaaccttttggaaatgcattcatctcaatgtgatcgtttattattgaaatctcctattttgcattggaatgagttccaagctttgaaaaacattgaagctgcttacccatcatggtctgtagcagaaattgatatcacattcgacaagagtgagggtctattgggctataccgacacaattgataaaatcactaagttagcgagcgaagcaattgatgatggtaaaaagatcttaataattactgacaggaaaatgggtgccaaccgtgtttccatctcctctttgattgcaatttcatgtattcatcatcacctaatcagaaacaagcagcgttcccaagttgctttgattttggaaacaggtgaagccagagaaattcaccatttctgtgtcctactaggttatggttgtgatggtgtttatccatacttagccatggaaactttggtcagaatgaatagagaaggtctacttcgtaatgtcaacaatgacaatgatacacttgaggaagggcaaatactagaaaattacaagcacgctattgatgcaggtatcttgaaggttatgtctaaaatgggtatctccactctagcatcctacaaaggtgctcaaatttttgaagccctaggtttagataactctattgttgatttgtgtttcacaggtacttcttccagaattagaggtgtaactttcgagtatttggctcaagatgccttttctttacatgagcgtggttatccatccagacaaaccattagtaaatctgttaacttaccagaaagtggtgaataccactttagggatggtggttacaaacacgtcaacgaaccaaccgcaattgcttcgttacaagatactgtcagaaacaaaaatgatgtctcttggcaattatatgtaaagaaggaaatggaagcaattagagactgtacactaagaggactgttagaattagattttgaaaattctgtcagtatccctctagaacaagttgaaccatggactgaaattgccagaagatttgcgtcaggtgcaatgtcttatggttctatttctatggaagctcactctacattggctattgccatgaatcgtttaggggccaaatccaattgtggtgaaggtggtgaagacgcagaacgttctgctgttcaagaaaacggtgatactatgagatctgctatcaaacaagttgcttccgctagattcggtgtaacttcatactacttgtcagatgctgatgaaatccaaattaagattgctcagggtgctaagccgggtgaaggtggtgaactaccagcccacaaagtgtctaaggatatcgcaaaaaccaggcactccacccctaatgttgggttaatctctcctcctcctcatcacgatatttattccattgaagatttgaaacaactgatttatgatttgaaatgtgctaatccaagagcgggaatttctgtaaagttggtttccgaagttggtgttggtattgttgcctctggtgtagctaaggctaaagccgatcatatcttagtttctggtcatgatggtggtacaggtgctgcaagatggacgagtgtcaaatatgcaggtttgccatgggaattaggtctagctgaaactcaccagactttagtcttgaatgatttaagacgtaatgttgttgtccaaaccgatggtcaattgagaactgggtttgatattgctgttgcagttttattaggggcagaatcttttaccttggcaacagttccattaattgctatgggttgtgttatgttaagaagatgtcacttgaactcttgtgctgttggtattgccacacaagatccatatttgagaagtaagtttaagggtcagcccgaacatgttatcaacttcttctattacttgatccaagatttaagacaaatcatggccaagttaggattccgtaccattgacgaaatggtgggccattctgaaaaattaaagaaaagggacgacgtaaatgccaaagccataaatatcgatttatctcctattttgaccccagcacatgttattcgtccaggtgttccaaccaagttcactaagaaacaagaccacaaactccacacccgtctagataataagttaatcgatgaggctgaagttactttggatcgtggcttaccagtgaatattgacgcctctataatcaatactgatcgtgcactcggttctactttatcttacagagtctcgaagaaatttggtgaagatggtttgccaaaggacaccgttgtcgttaacatagaaggttcagcgggtcaatcttttggtgctttcctagcttctggtatcacttttatcttgaatggtgatgctaatgattatgttggtaaaggtttatccggtggtattattgtcattaaaccaccaaaggattctaaattcaagagtgatgaaaatgtaattgttggtaacacttgtttctatggtgctacttctggtactgcattcatttcaggtagtgccggtgagcgtttcggtgtcagaaactctggtgccaccatcgttgttgagagaattaagggtaacaatgcctttgagtatatgactggtggtcgtgccattgtcttatcacaaatggaatccctaaacgccttctctggtgctactggtggtattgcatactgtttaacttccgattacgacgattttgttggaaagattaacaaagatactgttgagttagaatcattatgtgacccggtcgagattgcgtttgttaagaatttgatccaggagcattggaactacacacaatctgatctagcagccaggattctcggtaatttcaaccattatttgaaagatttcgttaaagtcattccaactgattataagaaagttttgttgaaggagaaagcagaagctaccaaggcaaaggctaaggcaacttcagaatacttaaagaagtttagatcgaaccaagaagttgatgacgaagtcaatactctattaattgctaatcaaaaagctaaagagcaagaaaagaagaagagtattactatttcaaataaggccactttgaaggagcctaaggttgttgatttagaagatgcagttccagattccaaacagctagagaagaatagcgaaaggattgaaaaaacacgtggttttatgatccacaaacgtcgtcatgagacacacagagatccaagaaccagagttaatgactggaaagaatttactaatcctattaccaagaaggatgccaaatatcaaactgcgagatgtatggattgtggtacaccattctgtttgtctgataccggttgtcccctatctaacattatccccaagtttaatgaattgttattcaagaaccaatggaagttggcactggacaaattgctagagacaaacaatttcccagaattcactggaagagtatgtccagcaccctgtgagggagcttgtacactaggtattattgaagacccagtgggcataaaatcggttgaaagaattatcattgacaatgctttcaaggaaggatggattaagccttgtccaccaagcacacgcactggctttacagtgggtgtcattggttctggtccagcaggtttagcgtgtgctgatatgttgaaccgtgccggacacacggtcactgtttatgaaagatccgaccgttgtggtgggttattgatgtacggtattccaaacatgaagttggataaggctatagtgcaacgtcgtattgatctattgagtgccgaaggtattgactttgttaccaacaccgaaattggtaaaaccataagcatggatgagctaaagaacaagcacaatgcagtagtgtatgctatcggttctaccattccacgtgacttacctattaagggtcgtgaattgaagaatattgattttgccatgcagttgttggaatctaacacaaaagctttattgaacaaagatctggaaatcattcgtgaaaagatccaaggtaagaaagtaattgttgtcggtggtggtgacacaggtaacgattgtttaggtacatctgtaagacacggtgcagcatcagttttgaatttcgaattgttgtctgagccaccagtggaacgtgccaaagacaatccatggcctcaatggccgcgtgtcatgagagtggactacggtcatgctgaagtgaaggagcattatggtagagaccctcgtgaatactgcatcttgtccaaggaatttatcggtaacgatgagggtgaagtcactgctatcagaactgtgcgcgtagaatggaagaagtcacaaagtggcgtatggcaaatggtagaaattcccaacagtgaagagatctttgaagccgatatcattttgttgtccatgggtttcgtgggtcctgaattgatcaatggcaacgataacgaagttaagaagacaagacgtggtacgattgccacactcgacgactcctcatactctattgatggaggaaagacttttgcatgtggtgactgtagaagagggcaatctttgattgtctgggccatccaagaaggtagaaaatgtgctgcctctgtcgataagttcctaatggacggcactacgtatctaccaagtaatggtggtatcgttcaacgtgattacaaactattgaaagaattagctagtcaagtctaa

SEQ ID NO: 43 - protein, 2145 aa, Saccharomyces cerevisiae
MPVLKSDNFDPLEEAYEGGTIQNYNDEHHLHKSWANVIPDKRGLYDPDYEHDACGVGFVANKHGEQSHKIVTDARYLLVNMTHRGAVSSDGNGDGAGILLGIPHEFMKREFKLDLDLDIPEMGKYAVGNVFFKKNEKNNKKNLIKCQKIFEDLAASFNLSVLGWRNVPVDSTILGDVALSREPTILQPLLVPLYDEKQPEFNETKFRTQLYLLRKEASLQIGLENWFYVCSLNNTTIVYKGQLTPAQVYNYYPDLTNAHFKSHMALVHSRFSTNTFPSWDRAQPLRWLAHNGEINTLRGNKNWMRSREGVMNSATFKDELDKLYPIIEEGGSDSAALDNVLELLTINGTLSLPEAVMMMVPEAYHKDMDSDLKAWYDWAACLMEPWDGPALLTFTDGRYCGAILDRNGLRPCRYYITSDDRVICASEVGVIPIENSLVVQKGKLKPGDLFLVDTQLGEMVDTKKLKSQISKRQDFKSWLSKVIKLDDLLSKTANLVPKEFISQDSLSLKVQSDPRLLANGYTFEQVTFLLTPMALTGKEALGSMGNDAPLACLNENPVLLYDYFRQLFAQVTNPPIDPIREANVMSLECYVGPQGNLLEMHSSQCDRLLLKSPILHWNEFQALKNIEAAYPSWSVAEIDITFDKSEGLLGYTDTIDKITKLASEAIDDGKKILIITDRKMGANRVSISSLIAISCIHHHLIRNKQRSQVALILETGEAREIHHFCVLLGYGCDGVYPYLAMETLVRMNREGLLRNVNNDNDTLEEGQILENYKHAIDAGILKVMSKMGISTLASYKGAQIFEALGLDNSIVDLCFTGTSSRIRGVTFEYLAQDAFSLHERGYPSRQTISKSVNLPESGEYHFRDGGYKHVNEPTAIASLQDTVRNKNDVSWQLYVKKEMEAIRDCTLRGLLELDFENSVSIPLEQVEPWTEIARRFASGAMSYGSISMEAHSTLAIAMNRLGAKSNCGEGGEDAERSAVQENGDTMRSAIKQVASARFGVTSYYLSDADEIQIKIAQGAKPGEGGELPAHKVSKDIAKTRHSTPNVGLISPPPHHDIYSIEDLKQLIYDLKCANPRAGISVKLVSEVGVGIVASGVAKAKADHILVSGHDGGTGAARWTSVKYAGLPWELGLAETHQTLVLNDLRRNVVVQTDGQLRTGFDIAVAVLLGAESFTLATVPLIAMGCVMLRRCHLNSCAVGIATQDPYLRSKFKGQPEHVINFFYYLIQDLRQIMAKLGFRTIDEMVGHSEKLKKRDDVNAKAINIDLSPILTPAHVIRPGVPTKFTKKQDHKLHTRLDNKLIDEAEVTLDRGLPVNIDASIINTDRALGSTLSYRVSKKFGEDGLPKDTVVVNIEGSAGQSFGAFLASGITFILNGDANDYVGKGLSGGIIVIKPPKDSKFKSDENVIVGNTCFYGATSGTAFISGSAGERFGVRNSGATIVVERIKGNNAFEYMTGGRAIVLSQMESLNAFSGATGGIAYCLTSDYDDFVGKINKDTVELESLCDPVEIAFVKNLIQEHWNYTQSDLAARILGNFNHYLKDFVKVIPTDYKKVLLKEKAEATKAKAKATSEYLKKFRSNQEVDDEVNTLLIANQKAKEQEKKKSITISNKATLKEPKVVDLEDAVPDSKQLEKNSERIEKTRGFMIHKRRHETHRDPRTRVNDWKEFTNPITKKDAKYQTARCMDCGTPFCLSDTGCPLSNIIPKFNELLFKNQWKLALDKLLETNNFPEFTGRVCPAPCEGACTLGIIEDPVGIKSVERIIIDNAFKEGWIKPCPPSTRTGFTVGVIGSGPAGLACADMLNRAGHTVTVYERSDRCGGLLMYGIPNMKLDKAIVQRRIDLLSAEGIDFVTNTEIGKTISMDELKNKHNAVVYAIGSTIPRDLPIKGRELKNIDFAMQLLESNTKALLNKDLEIIREKIQGKKVIVVGGGDTGNDCLGTSVRHGAASVLNFELLSEPPVERAKDNPWPQWPRVMRVDYGHAEVKEHYGRDPREYCILSKEFIGNDEGEVTAIRTVRVEWKKSQSGVWQMVEIPNSEEIFEADIILLSMGFVGPELINGNDNEVKKTRRGTIATLDDSSYSIDGGKTFACGDCRRGQSLIVWAIQEGRKCAASVDKFLMDGTTYLPSNGGIVQRDYKLLKELASQV

SEQ ID NO: 44 - DNA, 1113 bp, Saccharomyces cerevisiae
  source 1..1113: mol_type = other DNA
atggctgaagcaagcatcgaaaagactcaaattttacaaaaatatctagaactggaccaaagaggtagaataattgccgaatacgtttggatcgatggtactggtaacttacgttccaaaggtagaactttgaagaagagaatcacatccattgaccaattgccagaatggaacttcgacggttcttctaccaaccaagcgccaggccacgactctgacatctatttgaaacccgttgcttactacccagatcctttcaggagaggtgacaacattgttgtcttggccgcatgttacaacaatgacggtactccaaacaagttcaaccacagacacgaagctgccaagctatttgctgctcataaggatgaagaaatctggtttggtctagaacaagaatacactctatttgacatgtatgacgatgtttacggatggccaaagggtgggtacccagctccacaaggtccttactactgtggtgttggtgccggtaaggtttatgccagagacatgatcgaagctcactacagagcttgtttgtatgccggattagaaatttctggtattaacgctgaagttatgccatctcaatgggaattccaagtcggtccatgtaccggtattgacatgggtgaccaattatggatggccagatactttttgcacagagtggcagaagagtttggtatcaagatctcattccatccaaagccattgaagggtgactggaacggtgccggttgtcacactaacgtttccaccaaggaaatgagacaaccaggtggtatgaaatacatcgaacaagccatcgagaagttatccaagagacacgctgaacacattaagttgtacggtagcgataacgacatgagattaactggtagacatgaaaccgcttccatgactgccttttcttctggtgtcgccaacagaggtagctcaattagaatcccaagatccgtcgccaaggaaggttacggttactttgaagaccgtagaccagcttccaacatcgacccatacttggttacaggtatcatgtgtgaaactgtttgcggtgctattgacaatgctgacatgacgaaggaatttgaaagagaatcttcataa

SEQ ID NO: 45 - protein, 370 aa, Saccharomyces cerevisiae
MAEASIEKTQILQKYLELDQRGRIIAEYVWIDGTGNLRSKGRTLKKRITSIDQLPEWNFDGSSTNQAPGHDSDIYLKPVAYYPDPFRRGDNIVVLAACYNNDGTPNKFNHRHEAAKLFAAHKDEEIWFGLEQEYTLFDMYDDVYGWPKGGYPAPQGPYYCGVGAGKVYARDMIEAHYRACLYAGLEISGINAEVMPSQWEFQVGPCTGIDMGDQLWMARYFLHRVAEEFGIKISFHPKPLKGDWNGAGCHTNVSTKEMRQPGGMKYIEQAIEKLSKRHAEHIKLYGSDNDMRLTGRHETASMTAFSSGVANRGSSIRIPRSVAKEGYGYFEDRRPASNIDPYLVTGIMCETVCGAIDNADMTKEFERESS

SEQ ID NO: 46 - DNA, 1431 bp, Lactobacillus delbrueckii
  source 1..1431: mol_type = other DNA
atgaccgaacactacttgaactacgttaatggtgaatggcgtgattctgctgatgccattgaaatttttgaaccagctactggtaagtccttgggtactgttccagctatgtctcatgaagatgttgattacgttatgaactccgctaaaaaagctttgccagcttggagagctttgtcttatgttgaaagagctgcttacttgcaaaaggctgctgatatcttgtatagagatgccgaaaagattggctccaccttgtctaaagaaattgccaagggtttgaagtcctccattggtgaagttactagaactgctgaaatcgttgagtacactgctaaagttggtgttactttggatggtgaagtaatggaaggtggtaattttgaagctgcctctaaaaacaagttggccgttgttagaagagaaccagttggtttggttttggctatttctccattcaactacccagttaatttggccggttctaaaattgctccagctttgatgggtggtaatgttgttgcttttaaaccaccaactcagggttctatttctggtttgttgttagctaaggcttttgctgaagctggtttgcctgctggtgtttttaacactattactggtagaggtagagtcatcggtgattacatcgttgaacatccagctgttaacttcatcaactttactggttcttctgccgttggtaagaatattggtaaattggctggtatgaggccaatcatgttggaattaggtggtaaagatgctgccatcgttttggaagatgctgatttggatttgaccgctaagaatatcgttgctggtgcttttggttattctggtcaaagatgtactgctgttaagcgtgttttggtcatggattctgttgctgatgaattggttgaaaaggttactgctttggccaaggatttgactgttggtattcctgaagaagatgccgatattactccattgattgataccaagtctgccgattatgttcagggtttgattgaagaggctgcagaaaaaggtgctaaacctttgtttgacttcaagagagaaggcaacttgatctacccaatggttatggatcaagttaccaccgatatgagattggcttgggaagaaccatttggtccagttttgcctttcatcagagttaagtcagctgatgaagctgttatgattgccaacgaatctgaatatggcttgcagtcctctgttttctctagaaattttgaaaaggctttcgccattgccggtaagttggaagttggtacagttcatattaacaacaagacccaaagaggtccagataactttccatttttgggtgttaagtcatctggtgccggtgttcaaggtgtcaaatattctattcaagctatgaccagagtcaagtccgttgttttcaacatcgaagattaa

SEQ ID NO: 47 - protein, 476 aa, Lactobacillus delbrueckii
MTEHYLNYVNGEWRDSADAIEIFEPATGKSLGTVPAMSHEDVDYVMNSAKKALPAWRALSYVERAAYLQKAADILYRDAEKIGSTLSKEIAKGLKSSIGEVTRTAEIVEYTAKVGVTLDGEVMEGGNFEAASKNKLAVVRREPVGLVLAISPFNYPVNLAGSKIAPALMGGNVVAFKPPTQGSISGLLLAKAFAEAGLPAGVFNTITGRGRVIGDYIVEHPAVNFINFTGSSAVGKNIGKLAGMRPIMLELGGKDAAIVLEDADLDLTAKNIVAGAFGYSGQRCTAVKRVLVMDSVADELVEKVTALAKDLTVGIPEEDADITPLIDTKSADYVQGLIEEAAEKGAKPLFDFKREGNLIYPMVMDQVTTDMRLAWEEPFGPVLPFIRVKSADEAVMIANESEYGLQSSVFSRNFEKAFAIAGKLEVGTVHINNKTQRGPDNFPFLGVKSSGAGVQGVKYSIQAMTRVKSVVFNIED

SEQ ID NO: 48 - DNA, 1434 bp, Streptococcus thermophilus
  source 1..1434: mol_type = other DNA
atggctaagcagtacaagaactacgttaacggtgaatggaaaacctccgaaaactctattactatctacgctccagctaatggtgaagaattgggttctgttccagctatgtctcaagctgaagttgatgaagtttatgctgctgctaaagctgctttgccagcttggagagctttgtcttatgctgaaagagctgcttacttgcataaggctgctgatattttggaaagagatgccgaaaagatcggtcaggttttgtctaaagaaatctccaagggtttgaagtccgctattggtgaagttgttagaaccgccgaaattattcattacgctgctgaagaaggtttgaggttggaaggtgaagtattagaaggtggtgcttttgatgctggttccaaaaaaaagattgccgtcgttagaagagaaccagttggtttggttttggctatttctccattcaactacccagttaatttggccggttcaaaaattgctccagctttgattgctggtgatgttgttgcttttaaaccaccaactcaaggttccatttctggtttgttgttggttgaagcttttgtcgaagctggtattccagctggtgttttgaattctattactggtagaggttccgttatcggtgattatatcgttgaacacaaggccgttgatttcattaacttcactggttctactccagtcggtgaaaacattggtagattggctgctatgaggccagttatgttggaattaggtggtaaagatgctgccatcgttttggaagatgctgatttggatttgaccgctaagaatatcgttgctggtgctttcgattattctggtcaaagatgtactgccattaagcgtgttttggttatggattctgttgccgatgaattggttgaaaaggttactgctttggttggtaacattactgttggtatgccagaagaatctgcttctgttactccattgattgataccaaagctgccgattttgttcaaggtttgattgatgatgctgttgaacaaggtgctactgctaaaactgaattgaagagagaaggcaacttgatctacccagctgtttttgatcatgttaccaccgatatgagattggcttgggaagaaccatttggtccagttttgccttttatcagagtttcctctgttgaagaagccatcaagatctctaacgaatctgaattcggtttacaaggtgccgttttcactcaagattatccaagagcttttgccattgccgaacaattggaagttggtactgttcacattaacaacaagacccaaagaggtactgataactttccattcttgggtgtaaaaggttctggtgctggtactcaaggtgttaagtattctattgaagctatgaccagagtcaagtccaccgtttttgatatctctgactactaa

SEQ ID NO: 49 - protein, 477 aa, Streptococcus thermophilus
MAKQYKNYVNGEWKTSENSITIYAPANGEELGSVPAMSQAEVDEVYAAAKAALPAWRALSYAERAAYLHKAADILERDAEKIGQVLSKEISKGLKSAIGEVVRTAEIIHYAAEEGLRLEGEVLEGGAFDAGSKKKIAVVRREPVGLVLAISPFNYPVNLAGSKIAPALIAGDVVAFKPPTQGSISGLLLVEAFVEAGIPAGVLNSITGRGSVIGDYIVEHKAVDFINFTGSTPVGENIGRLAAMRPVMLELGGKDAAIVLEDADLDLTAKNIVAGAFDYSGQRCTAIKRVLVMDSVADELVEKVTALVGNITVGMPEESASVTPLIDTKAADFVQGLIDDAVEQGATAKTELKREGNLIYPAVFDHVTTDMRLAWEEPFGPVLPFIRVSSVEEAIKISNESEFGLQGAVFTQDYPRAFAIAEQLEVGTVHINNKTQRGTDNFPFLGVKGSGAGTQGVKYSIEAMTRVKSTVFDISDY

SEQ ID NO: 50 - DNA, 1428 bp, Streptococcus macacae
  source 1..1428: mol_type = other DNA
atgaccaagcagtacaaggattacgttaatggtgaatggaagctgtccaagaacgatatcaaaatctatgaaccagcttccggtgctgaattgggtttagttccagctatgtctactgaagaggttgattatgtttatgcttccgctcataaggctttgaaagaatggcgtgctttgtcttatgttgaaagagctgcttacttgcataaggttgccgatattttggaaagagatgccgaaaaaattggtgccgtcttgtctaaagaagttgctaaaggttacaagtccgccgtttctgaagttattagaaccgctgaaattatcaactacgctgctgaagagggtttgagaatggaaggtgaagttttggaaggtggttcttttgaagctgcttccaaaaagaagattgccgttgttagaagagaaccagttggtttggttttggctatttctccattcaactacccagttaatttggccggttctaaaattgctccagctttgattgctggtaacgttgttgcttttaaaccaccaactcaaggttccatttctggtttgttgttggctgaagcttttgctgaagcaggtttgccagctggtgtttttaacactattactggtagaggttccgaaatcggtgattacatcgttgaacatccagctgttaacttcatcaacttcactggttctactccaatcggtgaaagaattggtagaatggctggtatgaggccaatcatgttggaattaggtggtaaagattccgccattgtcttggaagatgctgatttggaattgaccgctaagaatatcgttgctggtgcttttggttattctggtcaaagatgtactgctgttaagcgtgttttagttatggaaggcgttgctgataagttggtcgaaaagattagagaaaaggttttggccttgaccattggtaatccagaaaacgatgctgatattaccccattgattgataccaaggctgctgattttgttgaaggtttgattaacgacgccaaagaaaagggtgctgataacttgactgaaatcaagagagaaggtaacttgatctgcccagttttgttcgataaggttactaccgatatgagattggcttgggaagaaccatttggtccagttttgccaattatcagagttaagtctgtcgaagaagccattgccatctctaatcaatctgaatacggtctgcaagcctctattttcactaatgattttccaagagctttcggtatcgccgaacaattggaagttggtactgttcatttgaacaacaagacccaaagaggtacagataactttccatttttgggcgctaaaaaatcaggtgctggtattcaaggtgtcaagtactctattgaagctatgactaccgtcaagtccgttgttttcgatatcaagtga

SEQ ID NO: 51 - protein, 475 aa, Streptococcus macacae
MTKQYKDYVNGEWKLSKNDIKIYEPASGAELGLVPAMSTEEVDYVYASAHKALKEWRALSYVERAAYLHKVADILERDAEKIGAVLSKEVAKGYKSAVSEVIRTAEIINYAAEEGLRMEGEVLEGGSFEAASKKKIAVVRREPVGLVLAISPFNYPVNLAGSKIAPALIAGNVVAFKPPTQGSISGLLLAEAFAEAGLPAGVFNTITGRGSEIGDYIVEHPAVNFINFTGSTPIGERIGRMAGMRPIMLELGGKDSAIVLEDADLELTAKNIVAGAFGYSGQRCTAVKRVLVMEGVADKLVEKIREKVLALTIGNPENDADITPLIDTKAADFVEGLINDAKEKGADNLTEIKREGNLICPVLFDKVTTDMRLAWEEPFGPVLPIIRVKSVEEAIAISNQSEYGLQASIFTNDFPRAFGIAEQLEVGTVHLNNKTQRGTDNFPFLGAKKSGAGIQGVKYSIEAMTTVKSVVFDIK

SEQ ID NO: 52 - DNA, 1431 bp, Streptococcus hyointestinalis
  source 1..1431: mol_type = other DNA
atgaccaaggcttacaagaactacgttaatggtgaatggaagctgtccgaagaatccattgaaatttttgctccagctaccggtgaatctttgggtactgttccagctatgactactgctgaagttgatgaagtttacgctaaagctaaagctgctcaaccagcttggagagctttgtcttatgttgaaagagctgcttacttgcataaggttgccgatattttggttagagatgccgaaaaaattggtgccgtcttgtctaaagaaattgctaagggttacaagtccgccgtttctgaagttattagaaccgctgaaattatcaactacgctgctgaagagggtttgagaatggaaggtgaagttttggaaggtggttcttttgaagctgcttccaaaaacaagattgccatcgttagaagagaaccagttggtttggttttggctatttctccattcaactacccaatcaatttggccggttctaaaattgctcctgctttgatttctggtaacgttgttgctttgaaaccaccaactcaaggttctatttctggtttgttgttggctgaagcttttgctgaagctggtttgccagctggtgtttttaacactattactggtagaggttccgttatcggtgattacatcgttgaacatgaagccgtcaatttcattaacttcactggttctactccaatcggtgaaagaattggtaaattggctggtatgaggccaatcatgttggaattaggtggtaaagattccgccattgtcttggaagatgctgatttggatttgaccgccaagaacattattgctggtgcttttggttattccggtcaaagatgtactgctgttaagagggtattagttatggattccgttgccgatgaattggtcgaaaagattagacaacaagtcttggacttgaccattggtaatcctgaagatgatgctgatattaccccattgattgataagaatgctgccgattttgtcgaaggtttgattaacgatgcttctgataagggtgcagaagctttgactgaaatcaagagagaaggtaacttgatctgcccagttttgttcgataaggttactaccgatatgagattggcttgggaagaaccatttggtccagttttgccaattatcagagttaagtctgttgaagaagccatcgagatctctaacaaatccgaatatggtctgcaagcttctgttttcactaacaattttccactggccttcaagatcgcttctcaattggaagttggtactgtccatattaacaacaagacccaaagaggtactgacaactttccatttttgggtgctaaaaaatcaggtgctggtgttcaaggtgttaagtactctattgaagctatgacctctgtcaagtccgttgtttttgatattgccaagtaa

SEQ ID NO: 53 - protein, 476 aa, Streptococcus hyointestinalis
MTKAYKNYVNGEWKLSEESIEIFAPATGESLGTVPAMTTAEVDEVYAKAKAAQPAWRALSYVERAAYLHKVADILVRDAEKIGAVLSKEIAKGYKSAVSEVIRTAEIINYAAEEGLRMEGEVLEGGSFEAASKNKIAIVRREPVGLVLAISPFNYPINLAGSKIAPALISGNVVALKPPTQGSISGLLLAEAFAEAGLPAGVFNTITGRGSVIGDYIVEHEAVNFINFTGSTPIGERIGKLAGMRPIMLELGGKDSAIVLEDADLDLTAKNIIAGAFGYSGQRCTAVKRVLVMDSVADELVEKIRQQVLDLTIGNPEDDADITPLIDKNAADFVEGLINDASDKGAEALTEIKREGNLICPVLFDKVTTDMRLAWEEPFGPVLPIIRVKSVEEAIEISNKSEYGLQASVFTNNFPLAFKIASQLEVGTVHINNKTQRGTDNFPFLGAKKSGAGVQGVKYSIEAMTSVKSVVFDIAK

SEQ ID NO: 54 - DNA, 1428 bp, Streptococcus urinalis
  source 1..1428: mol_type = other DNA
atgaccaagcagtacaagaactacgttaatggtgaatggaagttgtccgaaaacgagattaagatatatgctccagcttccggtgaagaattgggttctgttccagctatgactcaagctgaagttgatgatgtttacgcttctgctaaagctgctttgccagcttggagagctttgtcttatgttgaaagagctaactacttgcataaggccgctgatattttggttagagatgctgaaaagatcggctccgttttgtctcaagaagttgctaaaggtcataagtccgctgtttccgaagttattagaaccgctgaaattatcaactacgctgctgaagagggtttgagaatggaaggtgaagttttggaaggtggttcttttgaagctgcttccaaaaagaagattgccatcgttagaagagaaccagttggtttggttttggctatttctccattcaactacccagttaatttggccggttctaaaattgctccagcattgattgctggtaacgttgttgctttgaaaccaccaactcaaggttccatttctggtattttgttggctcaagcttttgctgaagctggtattccagctggtgttttcaacactattactggtagaggttccgttatcggtgattacatcgttgaacatgaagccgttaacttcatcaatttcactggttctactccagtcggtgaaagaataggtaaattggctggtatgaggccaatcatgttggaattaggtggtaaagattccgccattgtcttggaagatgctgatttggatgttgctgctaagaatatcgttgctggtgcttttggttattctggtcaaagatgtactgctgttaagcgtgttttggtcatggattctgttgctgatgcattggttgaaaaggtgtctcaaaaggtttctgctttgactattggtaaccctgaagatgatgctgatattaccccattgattgataccaaggctgctgattttgttgaaggtttgattaacgacgccaaagaaaaaggtgcacaaccattgcacgaaatcaagagagaaggtaatttggtttgcccattggttttcgataaggttactaccgatatgagattggcttgggaagaaccatttggtccagttttgcctttcatcagagttaagtctgttgaagaagccatcaagatctccaacgaatctgaatatggtctgcaagcttctgttttcactaacaattttccaagagctttcgccattgccgaacaattggaagttggtactgttcacattaacaacaagacccaaagaggtactgataactttccatttttgggcgctaaaaaatctggtgctggtgttcaaggtgttaagtactctattgaagctatgacctctgtcaagtccgttgtttttgatatcgagtaa

SEQ ID NO: 55 - protein, 475 aa, Streptococcus urinalis
MTKQYKNYVNGEWKLSENEIKIYAPASGEELGSVPAMTQAEVDDVYASAKAALPAWRALSYVERANYLHKAADILVRDAEKIGSVLSQEVAKGHKSAVSEVIRTAEIINYAAEEGLRMEGEVLEGGSFEAASKKKIAIVRREPVGLVLAISPFNYPVNLAGSKIAPALIAGNVVALKPPTQGSISGILLAQAFAEAGIPAGVFNTITGRGSVIGDYIVEHEAVNFINFTGSTPVGERIGKLAGMRPIMLELGGKDSAIVLEDADLDVAAKNIVAGAFGYSGQRCTAVKRVLVMDSVADALVEKVSQKVSALTIGNPEDDADITPLIDTKAADFVEGLINDAKEKGAQPLHEIKREGNLVCPLVFDKVTTDMRLAWEEPFGPVLPFIRVKSVEEAIKISNESEYGLQASVFTNNFPRAFAIAEQLEVGTVHINNKTQRGTDNFPFLGAKKSGAGVQGVKYSIEAMTSVKSVVFDIE

SEQ ID NO: 56 - DNA, 1428 bp, Streptococcus canis
  source 1..1428: mol_type = other DNA
atgactacccagtacaagaacttggttaatggtgaatggaagttgtccgaaaacgagattaagatatatgctccagctaccggtgaagaattgggttctgttccagctatgtctagagaagaggttgatgctgtttatggtgctgctagacaagctttggctggttggagagctttgtcttatgttgaaagagctgctttcttgcataaggctgctgatattttggttagagatgccgaaaagattggtgccatcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaattacccagttaatttggccggttcaaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaacttcactggttctactccaatcggtgaaagaataggtaaattggctggtatgaggccaatcatgttggaattaggtggtaaagattccgccattgtcttggaagatgctgatttggatttggctgctaagaatatcgttgctggtgcttttggttattctggtcaaagatgtactgctgttaagcgtgttttggtcatggaatctgttgctgatgatttggtcgaaaagatcagagataaggtcttgcaattgaccattggtaaccctgaagataacgctgatattacccctttgattgatacttctgctgccgattttgttgagggcttgattaaggatgctgttgataagggtgctactgctcatactgatattaagagagaaggtaacttgatctgcccaatcttgttcgatcatgttactaccgatatgagattggcttgggaagaaccatttggtccagttttgccaattattagagttgcctctgttgaagaagccatcaagatttctaacgaatctgaatacggtctgcaagcctctatttttactaccaattttccacaagctttcggtatcgctgaacaattggaagttggtactgttcacattaacaacaagacccaaagaggtactgataactttccatttttgggcgctaaaaaatctggtgctggtgttcaaggtgttaagtactctattgaagctatgacctccgttaagtccgttgttttcgatatccaatga

SEQ ID NO: 57 - protein, 475 aa, Streptococcus canis
MTTQYKNLVNGEWKLSENEIKIYAPATGEELGSVPAMSREEVDAVYGAARQALAGWRALSYVERAAFLHKAADILVRDAEKIGAILSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGERIGKLAGMRPIMLELGGKDSAIVLEDADLDLAAKNIVAGAFGYSGQRCTAVKRVLVMESVADDLVEKIRDKVLQLTIGNPEDNADITPLIDTSAADFVEGLIKDAVDKGATAHTDIKREGNLICPILFDHVTTDMRLAWEEPFGPVLPIIRVASVEEAIKISNESEYGLQASIFTTNFPQAFGIAEQLEVGTVHINNKTQRGTDNFPFLGAKKSGAGVQGVKYSIEAMTSVKSVVFDIQ

SEQ ID NO: 58 - DNA, 1428 bp, Streptococcus thoraltensis
  source 1..1428: mol_type = other DNA
atgtccaagcagtacaagaacttggttaatggtgaatggaagttgtccgacaacgaaatcaaaatctatgctccagctactggtgaagaattgggttctgttccagctatgtctcaagaagaggttgattacgtttacgaaactgctaaagctgctcaaccagcttggagagctttgtcttatgttgaaagagctgcttacttgcataaggttgccgatattttggatagagatgccgaaaagattggtgaggtcttgtctaaagaaattgccaaaggttacaaggctgccgtttctgaagttactagaactgctgatattatcagatacgctgctgaagagggtgttagaatgcaaggtgaagttttggaaggtggttcttttgatgctgcctccaaaaaaaagattgccatggttagaagagagccattgggtttagttttggctatttctccattcaactacccagttaatttggccggttctaaaattgctccagcattgatttctggtaacgttgttgctttgaaaccaccaactcaaggttctatttctggtttggttttagctgaagttttcgctgaagctggtattccagctggtgttttttctactattactggtagaggttccgttatcggtgattacatcgttgaacatgaagccgttaacttcatcaacttcactggttctactccagttggtgaaagaataggtaaaatggctggtatgaggccaatcatgttggaattaggtggtaaagattccgccattgtcttggaagatgctgatttggaagttgctgctaagaatatcgttgatggtgcttttggttactctggtcaaagatgtactgctgttaagcgtgttttggttatggattctgttgccgatgaattggtcgaaatgttgagagaaaaggtcttgaagttgactgttggtaaccctgaagataacgcagatattaccccattgattgatactgctgctgccgattttgttgaaggtttagttaatgatgccgttgaaaaaggtgctgatgctaagactgatatcttgagagaaggtaacttgatctacccaatcttgttcgataacgttactaccgatatgaagttggcttgggaagaaccatttggtccagttttgccagttatcagagtttcctctgttgaagaagccatcgaaatctctaacaaatctgaatacggtctgcaagcttccgttttcactaatgattttccattggctttctctatcgccgaacaattagaagttggtactgttcacattaacaacaagacccaaagaggtactgataactttccatttttgggcgctaaaaaatctggtgctggtactcaaggtgttaagtactctattgaagctatgaccaccgttaagtccgttgttttcgatatcaagtaa

SEQ ID NO: 59 - protein, 475 aa, Streptococcus thoraltensis
MSKQYKNLVNGEWKLSDNEIKIYAPATGEELGSVPAMSQEEVDYVYETAKAAQPAWRALSYVERAAYLHKVADILDRDAEKIGEVLSKEIAKGYKAAVSEVTRTADIIRYAAEEGVRMQGEVLEGGSFDAASKKKIAMVRREPLGLVLAISPFNYPVNLAGSKIAPALISGNVVALKPPTQGSISGLVLAEVFAEAGIPAGVFSTITGRGSVIGDYIVEHEAVNFINFTGSTPVGERIGKMAGMRPIMLELGGKDSAIVLEDADLEVAAKNIVDGAFGYSGQRCTAVKRVLVMDSVADELVEMLREKVLKLTVGNPEDNADITPLIDTAAADFVEGLVNDAVEKGADAKTDILREGNLIYPILFDNVTTDMKLAWEEPFGPVLPVIRVSSVEEAIEISNKSEYGLQASVFTNDFPLAFSIAEQLEVGTVHINNKTQRGTDNFPFLGAKKSGAGTQGVKYSIEAMTTVKSVVFDIK

SEQ ID NO: 60 - DNA, 1428 bp, Streptococcus dysgalactiae
  source 1..1428: mol_type = other DNA
atgactacccagtacaagaacttggttaatggtgattggaagttgtccgaatccgatattaagatatatgctccagctaccggtgaagaattgggttctgttccagctatgactcaagctgaagttgatgctgtttatgcttctgctaaaaaagctttgccagcttggagagctttgtcttatgttgaaagagctgcttacttgcataaggctgctgatattttggttagagatgccgaaaaaattggtgccgtcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaattacccagttaacttggccggttctaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaatttcactggttctactccaatcggtgaaggtattggtaaattggctggtatgaggccaatcatgttggaattaggtggtaaagattccgccattgtcttggaagatgctgatttggctttggctgctaagaatatcgttgctggtgcttttggttattctggtcaaagatgtactgctgttaagcgtgttttggtcatggataaggttgcagatcaattggctgctgaaatcaagactttggtcgaaaaattgtctgtcggtatgcctgaagatgatgcagatattactccattgattgataccaaggctgccgattttgttgaaggtttgattaaggatgctgcagataagggtgctactgctttgactacttttaacagagaaggcaacttgatctccccagttttgtttgatcatgttaccaccgatatgagattggcttgggaagaaccatttggtccagttttgccaattatcagagttacctctgttgaagaagccatcgaaatttctaacgcttccgaatatggtctgcaagcttctattttcactaacaactttccaaaggctttcggtattgccgaacaattagaagttggtactgttcacttgaacaacaagactcaaagaggtacagataacttcccatttttgggtgctaaaaagtctggtgctggtgttcaaggtgttaagtattctattgaagctatgaccaccgttaagtccgttgttttcgatatccaatga

SEQ ID NO: 61 - protein, 475 aa, Streptococcus dysgalactiae
MTTQYKNLVNGDWKLSESDIKIYAPATGEELGSVPAMTQAEVDAVYASAKKALPAWRALSYVERAAYLHKAADILVRDAEKIGAVLSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTKAADFVEGLIKDAADKGATALTTFNREGNLISPVLFDHVTTDMRLAWEEPFGPVLPIIRVTSVEEAIEISNASEYGLQASIFTNNFPKAFGIAEQLEVGTVHLNNKTQRGTDNFPFLGAKKSGAGVQGVKYSIEAMTTVKSVVFDIQ

SEQ ID NO: 62 - DNA, 750 bp, synthetic construct
  misc_feature 1..750: note = Mutated tsl1 promoter
  source 1..750: mol_type = other DNA
tctttcgatcactaccatgtctgtttaaccgagcaacgcgttcctccggagccgatggtactggctccggagaagggtcgttggtggcatccgagggcgccggtttggcatcatgttcggttcgcgagggtacttgcttggcgcccctgtgtttcacggtgtaaacaaacaagcacaccatcgccagtataaacactatagtcgatccatccatttttacttttgtgcgcgtaggtagccgtgcctcgcctgtgtgtgtgggaatgtctaaatgtgtcccgagttattgttctaaagcgggcaccattgtagtaacttattgcgaaatttctgctcttctcgtctcgctcaaaaatcgcgttcagggtaaaaggggcgaaacagagggccagatagaaatttcgagaaaagcgggtcacccccgcccctgcattttgatatggcgtatttgggattgcttgctcgaaagtgtctaagtccggctggcgggcctggcgccctcgccgaagggagataggaaggggcgggggtccgggcagcggctatggtgtcagttacctagggaaggagaagggggtagaaccaaggggctagcacactcaccctggggccctcgtctagccaagcttaaatataaatactaatgtaactataaatataaggatctaccgtgtcattgcacatccacccacccgtcgattaaaaaaccaaacaaagcaaagaatacaatagcaacgcaagatcaacaca

SEQ ID NO: 63 - DNA, 3297 bp, Saccharomyces cerevisiae
  source 1..3297: mol_type = other DNA
atggctctcatcgtggcatctttgtttttgccctaccaaccacaattcgagcttgacacctctctccctgagaactcgcaggtggactcatctctcgtgaacatccaggctatggccaatgaccaacagcaacaacgtgcgctttctaacaacatctcacaggaatcattggtcgcgccagcaccagaacaaggtgtcccaccagcaatctcaaggagtgccaccaggtcacccagtgctttcaaccgcgcctcgtctacgacaaatactgccactttagatgatcttgtctcttcggacatattcatggaaaacttgactgcgaatgcaactacctcacatacgccaacaagcaagactatacttaaaccccggaaaaatggttccgtggaacgattcttctccccttcttccaatattcccacggatcgcatcgcatcgccaatccagcatgagcatgactccggttcgagaattgcttcgccaatccaacagcaacagcaggaccccacggccaacttattaaagaacgtcaacaagtcattgttagtgcactcactgttgaacaacacctcacaaactagcctagaaggacccaacaaccacattgttaccccgaaatcgagggcgggcaacaggcctacttcggcggctacttctttagttaataggaccaaacaaggttcggcctcctctggatcttctgggtcttctgcgccaccttccattaaaaggattacgccccacttgactgcgtccgctgcaaaacagcgccccttattggctaaacagccttctaatctgaaatattcggagttagcagatatttcgtcgagtgagacgtcttcgcagcataatgagtcggacccggatgatctaactactgcccctgacgaggaatatgtttctgatttggaaatggatgacgcgaagcaggactacaaggttccaaagttcggcggctattccaataaatctaaacttaagaagtatgcgctgttaaggtcatctcaggagctgtttagccgtcttccatggtcgatcgttccctctatcaaaggtaatggcgccatgaagaacgccataaacactgcagtcttggagaatatcattccgcaccgtcatgttaagtgggtcggtaccgtcggaatcccaacggatgagattccggaaaatatccttgcgaacatctctgactctttaaaagacaagtacgactcctatcctgtccttacggacgacgtcaccttcaaagccgcatacaaaaactactgtaaacaaatcttgtggcctacgctgcattaccagattccagacaatccgaactcgaaggcttttgaagatcactcttggaagttctatagaaacttaaaccaaaggtttgcggacgcgatcgttaaaatccataagaaaggtgacaccatctggattcatgattaccatttaatgctggttccgcagatggtgagagacgtcttgccttttgccaaaataggatttaccttacatgtctcgttccccagtagtgaagtgtttaggtgtctggctcagcgtgagaagatcttagaaggcttgaccggtgcagactttgtcggcttccagacgagggagtatgcaagacatttcttacagacgtctaaccgtctgctaatggcggacgtggtacatgatgaagagctaaagtataacggcagagtcgtttctgtgaggttcaccccagttggtatagacgcctttgatttgcaatcgcaattgaaggatggaagtgtcatgcaatggcgtcaattgattcgtgaaagatggcaagggaaaaaactgattgtgtgtcgtgatcaattcgatagaattaggggtattcacaagaaattgttggcttatgaaaaatttttggtcgaaaatccagaatacgtggaaaaatcgactttaattcaaatctgtattggaagcagtaaggatgtagaactagaacgccagatcatgattgttgtggatagaatcaactcgctatccaccaatattagtatttctcaacctgtggtgtttttacatcaagatctagatttttctcagtatttagctttgagttcagaggcagatttgttcgtagtcagctctctaagggaaggtatgaacttgacatgtcacgaatttatcgtttgttctgaggacaaaaatgctcccctactgttgtcagaatttactggtagtgcatctttattgaatgatggcgctataataattaacccatgggataccaagaacttctcacaagccattctcaaggggttggagatgccattcgataagagaagaccacagtggaagaaattgatgaaagacattatcaacaacgactctacaaactggatcaaaacttctttacaagatattcatatttcgtggcaattcaatcaagaaggttccaagatcttcaaattgaatacaaaaacactgatggaagattaccagtcatctaaaaagcgtatgtttgttttcaacattgctgaaccaccttcatcgagaatgatttccatactgaatgacatgacttctaagggcaatatcgtttacataatgaactcatttccaaagcccattctggaaaatctttacagtcgggtgcaaaacattgggttgattgccgaaaatggtgcatacgttagtctgaacggtgtatggtacaacattgttgatcaagtcgattggcgtaacgatgtagccaaaattctcgaggacaaagtggaaagattacctggctcgtactacaagataaatgagtccatgatcaagttccacactgaaaatgcggaagatcaagatcgtgtagctagtgttatcggtgatgccatcacacatatcaatactgtttttgaccacaggggtattcatgcctacgtttacaaaaacgttgtttccgtacaacaagtgggactttccttatcggcagctcaatttcttttcagattctataattctgcttcagatccactggatacgagttccggccaaatcacaaatattcagacaccatctcaacaaaatccttcagatcaagaacaacaacctccagcctctcccactgtgtcgatgaaccatattgatttcgcatgtgtctctggttcatcgtctcctgtgcttgaaccattgttcaaattggtcaatgatgaagcaagtgaagggcaagtaaaagccggacacgccattgtttatggtgatgctacttctacttatgccaaagaacatgtaaatgggttaaacgaacttttcacgatcatttcaagaatcattgaagattaa

SEQ ID NO: 64 - protein, 1098 aa, Saccharomyces cerevisiae
MALIVASLFLPYQPQFELDTSLPENSQVDSSLVNIQAMANDQQQQRALSNNISQESLVAPAPEQGVPPAISRSATRSPSAFNRASSTTNTATLDDLVSSDIFMENLTANATTSHTPTSKTILKPRKNGSVERFFSPSSNIPTDRIASPIQHEHDSGSRIASPIQQQQQDPTANLLKNVNKSLLVHSLLNNTSQTSLEGPNNHIVTPKSRAGNRPTSAATSLVNRTKQGSASSGSSGSSAPPSIKRITPHLTASAAKQRPLLAKQPSNLKYSELADISSSETSSQHNESDPDDLTTAPDEEYVSDLEMDDAKQDYKVPKFGGYSNKSKLKKYALLRSSQELFSRLPWSIVPSIKGNGAMKNAINTAVLENIIPHRHVKWVGTVGIPTDEIPENILANISDSLKDKYDSYPVLTDDVTFKAAYKNYCKQILWPTLHYQIPDNPNSKAFEDHSWKFYRNLNQRFADAIVKIHKKGDTIWIHDYHLMLVPQMVRDVLPFAKIGFTLHVSFPSSEVFRCLAQREKILEGLTGADFVGFQTREYARHFLQTSNRLLMADVVHDEELKYNGRVVSVRFTPVGIDAFDLQSQLKDGSVMQWRQLIRERWQGKKLIVCRDQFDRIRGIHKKLLAYEKFLVENPEYVEKSTLIQICIGSSKDVELERQIMIVVDRINSLSTNISISQPVVFLHQDLDFSQYLALSSEADLFVVSSLREGMNLTCHEFIVCSEDKNAPLLLSEFTGSASLLNDGAIIINPWDTKNFSQAILKGLEMPFDKRRPQWKKLMKDIINNDSTNWIKTSLQDIHISWQFNQEGSKIFKLNTKTLMEDYQSSKKRMFVFNIAEPPSSRMISILNDMTSKGNIVYIMNSFPKPILENLYSRVQNIGLIAENGAYVSLNGVWYNIVDQVDWRNDVAKILEDKVERLPGSYYKINESMIKFHTENAEDQDRVASVIGDAITHINTVFDHRGIHAYVYKNVVSVQQVGLSLSAAQFLFRFYNSASDPLDTSSGQITNIQTPSQQNPSDQEQQPPASPTVSMNHIDFACVSGSSSPVLEPLFKLVNDEASEGQVKAGHAIVYGDATSTYAKEHVNGLNELFTIISRIIED

SEQ ID NO: 65 - DNA, 1083 bp, Entamoeba histolytica
  source 1..1083: mol_type = other DNA
atgaaaggacttgctatgcttggaattggaagaattggatggattgaaaagaaaatcccagaatgtggaccacttgatgcattagttagaccattagcacttgcaccatgtacatcagatacacataccgtttgggcaggagctattggagatagacatgatatgattcttggacatgaagcggttggacaaattgttaaagttggatcattagttaagagattaaaagttggagataaagttattgtaccagctattacaccagattggggagaagaagaatcgcaaagaggatatccaatgcattcaggaggaatgcttggaggatggaaattctcaaatttcaaggatggagttttttcagaagttttccatgttaatgaagcagatgccaatcttgcacttcttccaagagatattaaaccagaagatgcagttatgttatcagatatggtaactactggattccatggagcagaattagctaatattaaacttggagatactgtttgtgttattggtattggaccagttggattaatgtcagttgcaggagcaaaccatcttggagcaggaagaatctttgcagtaggatcaagaaaacattgttgtgatattgcattggaatatggagcaacagatattattaattataaaaatggagatattgtagaacaaattcttaaagctacagacggcaaaggagttgataaagtcgttattgcaggaggtgatgttcatacatttgcacaagcagtcaaaatgattaaaccaggatcagatattggaaatgttaattatcttggagaaggagataatattgatattccaagaagtgaatggggagttggaatgggtcataaacacattcatggaggtttaaccccaggtggaagagtcagaatggaaaaattagcatcacttatttcaactggtaaattagatacttctaaacttattacacatagatttgaaggattagaaaaagttgaagatgcattaatgttaatgaagaataaaccagcagaccttatcaaaccagttgtcagaattcattatgatgatgaagatactcttcattaa

SEQ ID NO: 66 - protein, 360 aa, Entamoeba histolytica
MKGLAMLGIGRIGWIEKKIPECGPLDALVRPLALAPCTSDTHTVWAGAIGDRHDMILGHEAVGQIVKVGSLVKRLKVGDKVIVPAITPDWGEEESQRGYPMHSGGMLGGWKFSNFKDGVFSEVFHVNEADANLALLPRDIKPEDAVMLSDMVTTGFHGAELANIKLGDTVCVIGIGPVGLMSVAGANHLGAGRIFAVGSRKHCCDIALEYGATDIINYKNGDIVEQILKATDGKGVDKVVIAGGDVHTFAQAVKMIKPGSDIGNVNYLGEGDNIDIPRSEWGVGMGHKHIHGGLTPGGRVRMEKLASLISTGKLDTSKLITHRFEGLEKVEDALMLMKNKPADLIKPVVRIHYDDEDTLH

SEQ ID NO: 67 - DNA, 1101 bp, Entamoeba nuttallii
  source 1..1101: mol_type = other DNA
atggaaggtaagactactatgaagggtttggctatgttgggtatcggtagaatcggttggatcgaaaagaagatcccagaatgtggtccattggacgctttggttagaccattggctttggctccatgtacttctgacactcacactgtttgggctggtgctatcggtgacagacacgacatgatcttgggtcacgaagctgttggtcaaatcgttaaggttggttctttggttaagagattgaaggttggtgacaaggttatcgttccagctatcactccagactggggtgaagaagaatctcaaagaggttacccaatgcactctggtggtatgttgggtggttggaagttctctaacttcaaggacggtgttttctctgaagttttccacgttaacgaagctgacgctaacttggctttgttgccaagagacatcaagccagaagacgctgttatgttgtctgacatggttactactggtttccacggtgctgaattggctaacatcaagttgggtgacactgtttgtgttatcggtatcggtccagttggtttgatgtctgttgctggtgctaaccacttgggtgctggtagaatcttcgctgttggttctagaaagcactgttgtgacatcgctttggaatacggtgctactgacatcatcaactacaagaacggtgacatcgttgaacaaatcttgaaggctactgacggtaagggtgttgacaaggttgttatcgctggtggtgacgttcacactttcgctcaagctgttaagatgatcaagccaggttctgacatcggtaacgttaactacttgggtgaaggtgacaacatcgacatcccaagatctgaatggggtgttggtatgggtcacaagcacatccacggtggtttgactccaggtggtagagttagaatggaaaagttggcttctttgatctctactggtaagttggacacttctaagttgatcactcacagattcgaaggtttggaaaaggttgaagacgctttgatgttgatgaagaacaagccagctgacttgatcaagccagttgttagaatccactacgacgacgaagacactttgcactaa

SEQ ID NO: 68 - protein, 366 aa, Entamoeba nuttallii
MEGKTTMKGLAMLGIGRIGWIEKKIPECGPLDALVRPLALAPCTSDTHTVWAGAIGDRHDMILGHEAVGQIVKVGSLVKRLKVGDKVIVPAITPDWGEEESQRGYPMHSGGMLGGWKFSNFKDGVFSEVFHVNEADANLALLPRDIKPEDAVMLSDMVTTGFHGAELANIKLGDTVCVIGIGPVGLMSVAGANHLGAGRIFAVGSRKHCCDIALEYGATDIINYKNGDIVEQILKATDGKGVDKVVIAGGDVHTFAQAVKMIKPGSDIGNVNYLGEGDNIDIPRSEWGVGMGHKHIHGGLTPGGRVRMEKLASLISTGKLDTSKLITHRFEGLEKVEDALMLMKNKPADLIKPVVRIHYDDEDTLH

SEQ ID NO: 69 - DNA, 1101 bp, Entamoeba dispar
  source 1..1101: mol_type = other DNA
atggaaggtaagactactatgaagggtttggctatgttgggtatcggtaagatcggttggatcgaaaagaagatcccagaatgtggtccattggacgctttggttagaccattggctttggctccatgtacttctgacactcacactgtttgggctggtgctatcggtgacagacacgacatgatcttgggtcacgaagctgttggtcaaatcgttaaggttggttctttggttaagagattgaaggttggtgacaaggttatcgttccagctatcactccagactggggtgaagaagaatctcaaagaggttacccaatgcactctggtggtatgttgggtggttggaagttctctaacttcaaggacggtgttttctctgaaatcttccacgttaacgaagctgacgctaacttggctttgttgccaagagacatcaaggctgaagacgctgttatgttgtctgacatggttactactggtttccacggtgctgaattggctaacatcaagttgggtgacactgtttgtgttatcggtatcggtccagttggtttgatgtctgttgctggtgctaaccacttgggtgctggtagaatcttcgctgttggttctagaaagcactgttgtgacatcgctatggaatacggtgctactgacatcatcaactacaagaacggtgacatcgttgaacaaatcttgaaggctactgacggtaagggtgttgacaaggttgttatcgctggtggtgacgttcacactttcgctcaagctgttaagatgatcaagccaggttctgacatcggtaacgttaactacttgggtgaaggtgacaacatcgacatcccaagatctgaatggggtgttggtatgggtcacaagcacatccacggtggtttgactccaggtggtagagttagaatggaaaagttggcttctttgatctctactggtaagttggacacttctaagttgatcactcacagattcgaaggtttggaaaaggttgaagacgctttgatgttgatgaagaacaagccagctgacttgatcaagccagttgttagaatccactacgacgacgaagacactttgcactaa

SEQ ID NO: 70 - protein, 366 aa, Entamoeba dispar
MEGKTTMKGLAMLGIGKIGWIEKKIPECGPLDALVRPLALAPCTSDTHTVWAGAIGDRHDMILGHEAVGQIVKVGSLVKRLKVGDKVIVPAITPDWGEEESQRGYPMHSGGMLGGWKFSNFKDGVFSEIFHVNEADANLALLPRDIKAEDAVMLSDMVTTGFHGAELANIKLGDTVCVIGIGPVGLMSVAGANHLGAGRIFAVGSRKHCCDIAMEYGATDIINYKNGDIVEQILKATDGKGVDKVVIAGGDVHTFAQAVKMIKPGSDIGNVNYLGEGDNIDIPRSEWGVGMGHKHIHGGLTPGGRVRMEKLASLISTGKLDTSKLITHRFEGLEKVEDALMLMKNKPADLIKPVVRIHYDDEDTLH

SEQ ID NO: 71 - DNA, 1428 bp, Streptococcus pyogenes
  source 1..1428: mol_type = other DNA
atggctaagcagtacaagaacttggttaatggtgaatggaagttgtccgaaaacgaaattactatctatgctccagctaccggtgaagaattgggttctgttccagctatgactcaagctgaagttgatgctgtttatgcttctgctaaaaaagctttgccagcttggagagctttgtcttatgttgaaagagctgcttacttgcataaggctgctgatattttggttagagatgccgaaaaaattggtgccgtcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaactacccagttaatttggccggttcaaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaatttcactggttctactccaatcggtgaaggtattggtaaattggctggtatgaggccaatcatgttggaattaggtggtaaagattccgccattgtcttggaagatgctgatttggctttggctgctaagaatatcgttgctggtgcttttggttattctggtcaaagatgtactgctgttaagcgtgttttggtcatggataaggttgcagatcaattggctgctgaaatcaagactttggtcgaaaaattgtctgtcggtatgcctgaagatgatgcagatattactccattgattgataccaaggctgccgattttgttgaaggtttgattaaggatgctaccgataagggtgctactgctttgactgcttttaatagagaaggcaacttgatctccccagttttgtttgatcatgttaccaccgatatgagattggcttgggaagaaccatttggtccagttttgccaattattagagttactactgtcgaagaggccatcaagatttctaacgaatctgaatacggtctgcaagcctctatttttactaccaattttccaaaggctttcggtatcgctgaacaattggaagttggtactgttcacttgaacaacaagactcaaagaggtacagataacttcccatttttgggtgctaaaaagtctggtgctggtgttcaaggtgttaagtattctattgaagctatgaccaccgttaagtccgttgttttcgatatccaatga

SEQ ID NO: 72 - protein, 475 aa, Streptococcus pyogenes
MAKQYKNLVNGEWKLSENEITIYAPATGEELGSVPAMTQAEVDAVYASAKKALPAWRALSYVERAAYLHKAADILVRDAEKIGAVLSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTKAADFVEGLIKDATDKGATALTAFNREGNLISPVLFDHVTTDMRLAWEEPFGPVLPIIRVTTVEEAIKISNESEYGLQASIFTTNFPKAFGIAEQLEVGTVHLNNKTQRGTDNFPFLGAKKSGAGVQGVKYSIEAMTTVKSVVFDIQ

SEQ ID NO: 73 - DNA, 1428 bp, Streptococcus ictaluri
  source 1..1428: mol_type = other DNA
atgaccaaagagtacaagaacttggttaacggtgaatggaagttgtccgataacaacattactatctacgaaccagctactggtaaagctttgggttctgttccagctatgtctcaagaagaggttgattacgtttacgcttctgctaaacaagctttgccaaaatggcgtgctttgtcttatgttgaaagagctgcttacttgcataaggctgctgatattttggttagagatgccgaaaagattggtgccatcttgtctaaagaagttgctaagggttttaaggctgccgtttctgaagttgttagaaccgctgaaattatcaactatgctgctgaagagggtttgagaatgcaaggtgaagttttggaaggtggttcttttgaagctgcttccaaaaaaaagatcgccatcgttagaagagaaccagttggtttggttttggctatttctccattcaactacccagttaatttggccggttctaaaattgctccagctttgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaacttcactggttctactgctattggtgaaggtattggtaaattggctggtatgaggccaatcatgttggaattaggtggtaaagattccgccattgtcttggaagatgctgatttggttttagctgctaagaacatagtttctggtgcttttggttactctggtcaaagatgtactgccgttaagagaatcttggttatggattctgttgctgatcaattggcctccgaaatcaagattttggtcgaacaattgtccgttggtatccctgaagaagatgcagatattactccattgattgataccaaggccgctgattttgttgaaggtttgattgatgatgctaaggctaaaggtgctttggctttgactgaatgtaagagagataacaacttgatctccccagttttgttcgatagagttactaccgatatgagattggcttgggaagaaccatttggtccagttttgcctttgatcagagtttcctctgttgaagaagccatcgaaatttctaacgcttccgaatatggtctgcaagcttctatttttaccaacaactttccacaagctttcgctatcgctgaacaattggaagttggtactgttcacttgaacaacaagactcaaagaggtacagataacttcccatttttgggtgctaaaaaatctggtgctggtgttcaaggtgttaagtactctattgaagctatgaccaacttgaagtccgttgttttcgatatcaagtaa

SEQ ID NO: 74 - protein, 475 aa, Streptococcus ictaluri
MTKEYKNLVNGEWKLSDNNITIYEPATGKALGSVPAMSQEEVDYVYASAKQALPKWRALSYVERAAYLHKAADILVRDAEKIGAILSKEVAKGFKAAVSEVVRTAEIINYAAEEGLRMQGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTAIGEGIGKLAGMRPIMLELGGKDSAIVLEDADLVLAAKNIVSGAFGYSGQRCTAVKRILVMDSVADQLASEIKILVEQLSVGIPEEDADITPLIDTKAADFVEGLIDDAKAKGALALTECKRDNNLISPVLFDRVTTDMRLAWEEPFGPVLPLIRVSSVEEAIEISNASEYGLQASIFTNNFPQAFAIAEQLEVGTVHLNNKTQRGTDNFPFLGAKKSGAGVQGVKYSIEAMTNLKSVVFDIK

SEQ ID NO: 75 - DNA, 1461 bp, Clostridium perfringens
  source 1..1461: mol_type = other DNA
atgttctcctgcatcaagggtaaagaaagaaccttcagaaacttgatcaacggtgaatggatcaactcctcatccgataagttcatcgatatctattctccagttggtaactgcttggttggtaaagttccagctatgactactgatgaagttgatttggctatcaagtccgctaaagaagctcaaaaggtttggagaaatgttccagttaacaagagagccgagatcttgtacaaagctgccgatattttgatcgaaaaggttgaagatatcgccgagatcatgatgagagaaattggtaaggataagaagtccgccgaatccgaaattttgagatctgctgattacattaagttcactgctgataccgctaagaacttgtctggtgaatctattccaggtgattcatttccaggtttcaagaggaacaagatttccttggttactagagaaccattgggtgttgttttggcaatttctccattcaactacccaatcaatttggccgcttctaaaattgctccagctttggttgctggtaattccgttgttttgaaaccagctactcaaggttcattgtgtggtctgtatttggctaaggtttttgaacaagctggtgttcctgctggtgttttgaatactgttactggtagaggttccgaaatcggtgattatatcgttacccatccagagatcgatttcattaactttactggttctaccgaagttggcaccagaatttctagaattactaccatggttcccttgttgatggaattaggtggtaaagatgctgctatcgttttggctgatgctgatttggatttggctgcttcaaatatcgttgctggtgcttattcttactctggtcaaagatgtactgccgtcaagagaattttggttgttaatgaagttgccgacaagttggtggaaaaggtcaaagaaaaggtccagaacttgaagattggtaacccattggaagaagatgttgatatcgttccattgatcgattctaaggctgctgattttgtttgggaattgattgatgatgccagagaaaaaggtgcccatttgttggttggtggtacaagagaagaaaacatgatctacccaactttgttcgataacgttaccaccgatatgagattggcttgggaagaaccatttggtccagttttgccaattatcagagttaaggacaaggatgaagccattgaaatcgctaacaaatctgaatacggcctgcaatcttctgttttcaccgaaaacattaacgaagctttctacgttgccgatagattggaagttggtactgttcaagtcaacaacaagactgaaagaggtcctgatcattttccattcttgggtgttaaggcttctggtattggtacacaaggtatcagatactccatcgaatctatgtctagaccaaaggctaccgttatcaacttggttagatga

SEQ ID NO: 76 - protein, 486 aa, Clostridium perfringens
MFSCIKGKERTFRNLINGEWINSSSDKFIDIYSPVGNCLVGKVPAMTTDEVDLAIKSAKEAQKVWRNVPVNKRAEILYKAADILIEKVEDIAEIMMREIGKDKKSAESEILRSADYIKFTADTAKNLSGESIPGDSFPGFKRNKISLVTREPLGVVLAISPFNYPINLAASKIAPALVAGNSVVLKPATQGSLCGLYLAKVFEQAGVPAGVLNTVTGRGSEIGDYIVTHPEIDFINFTGSTEVGTRISRITTMVPLLMELGGKDAAIVLADADLDLAASNIVAGAYSYSGQRCTAVKRILVVNEVADKLVEKVKEKVQNLKIGNPLEEDVDIVPLIDSKAADFVWELIDDAREKGAHLLVGGTREENMIYPTLFDNVTTDMRLAWEEPFGPVLPIIRVKDKDEAIEIANKSEYGLQSSVFTENINEAFYVADRLEVGTVQVNNKTERGPDHFPFLGVKASGIGTQGIRYSIESMSRPKATVINLVR

SEQ ID NO: 77 - DNA, 1461 bp, Clostridium chromiireducens
  source 1..1461: mol_type = other DNA
atgttcaactgcatcaagtgcgagaacaacaacttcaagaacttgattaacggtgaatgggttggtaacaaggataacaaggttatcgaaatctactccccattggacaattctttggttggtactgttccagctatgacccaagaagatattgatcacgttattcaagttgccaaggacggtcaaagagaatggtctaaagttccaatgaacgaaagagccgagatcttgtacaaagctgctgatattttggttgaaaacgccaacgaattggtcgacattatgattagagaaatcggcaaggaccgtaagagttccaaatctgaaattcatagaaccgccgacttcattagattcactgctgatactgctaagaacatggctggtgaatctattccaggtgatacttttccaggtttcaagaggaacaagatttccgttgttaacagagaaccattgggtgttgttttggctatttctccattcaactaccccattaacttgtccgcttctaaaattgctccagccattatcgttggtaactccgttgttttgaaaccagctactcaaggttctttgtgtggtctgtatttggctaaggttttccaagaggctggtgttccaaatggtgttttgaacactattactggtaagggttccgaaattggtgattatgctgttactcataagggcgtcaacttcattaactttactggttctactgaggtcggtgtcaagatttctaagattacttctatggtccccttgttgatggaattaggtggtaaagatgctgccatcgttttgaaagatgccgatttggatttggctgctaacaatatcgttgctggtggttattcttactctggtcaaagatgtactgccgtcaagagaattttggttttggaaaaggttgccgatgagttggtcaaaaaggtcaaagaaaagatgtccaacttgactgttggtaacccattggataaggatgttgatatcgttccattgatctctaccaagtctgctgatttcgttgaagagttgattaaggatgccattgataagggtgcagatttggttgttggtggtaaaagagatggtaacttgatctacccaaccttgttcgataatgttaccggtgatatgagaattgcttgggaagaaccatttggtccagttttgccaattatgagagttaaggacaaggatgaagccatcgaaattgctaacaagtccgaatatggtttacaaggtgctgttttcaccgaaaacattgaagatgctttctacgttgccgatagattggaagttggtacagttcaagttaacaacaagactgaaagaggtccagatcattttccattcttgggtgttaaggcttctggtattggtacacaaggtatcagatactccatcgaatctatgtctagaccaaaggctaccgttatcaacttggttagatga

SEQ ID NO: 78 - protein, 486 aa, Clostridium chromiireducens
MFNCIKCENNNFKNLINGEWVGNKDNKVIEIYSPLDNSLVGTVPAMTQEDIDHVIQVAKDGQREWSKVPMNERAEILYKAADILVENANELVDIMIREIGKDRKSSKSEIHRTADFIRFTADTAKNMAGESIPGDTFPGFKRNKISVVNREPLGVVLAISPFNYPINLSASKIAPAIIVGNSVVLKPATQGSLCGLYLAKVFQEAGVPNGVLNTITGKGSEIGDYAVTHKGVNFINFTGSTEVGVKISKITSMVPLLMELGGKDAAIVLKDADLDLAANNIVAGGYSYSGQRCTAVKRILVLEKVADELVKKVKEKMSNLTVGNPLDKDVDIVPLISTKSADFVEELIKDAIDKGADLVVGGKRDGNLIYPTLFDNVTGDMRIAWEEPFGPVLPIMRVKDKDEAIEIANKSEYGLQGAVFTENIEDAFYVADRLEVGTVQVNNKTERGPDHFPFLGVKASGIGTQGIRYSIESMSRPKATVINLVR

SEQ ID NO: 79 - DNA, 1461 bp, Clostridium botulinum
  source 1..1461: mol_type = other DNA
atgttcaaccacatcaaggacgaaaacaacaccttcaagaacttgattaacggtgagtgggtttcctctagatctttcgttgaaatcaagtcccctctgtctaattctttgttgggtagagttccagctatgaccaaagaagaagttgatattgctgttcagaccgctaaagaagctcaaaaaaagtggaacaagatcaccattaacgaaagggctgagatcttgtacaaagcctctgatattttgttggagaacatcgacgaactgtccgaattgatgatgatggaaattgccaaggatagaaagtcctgcagatctgaagtttctagaacctccgatttcattaagttcactgctgatactgccaagaatttgtccggtgaatctattccaggtgattctttcccaggtttcaaaaacaacaaggtgtccattgtcaaaagggaaccattgggtgttgtattggctatttctccattcaactaccccattaacttgtccgcttctaaaattgctccaggtttgatggctggtaactctgttgttttgaagccagctactcaaggttctttgtgtggtctatatttggccagaatttttgaaaaggctggtgttccagctggtgttttgaacactattactggtaagggttctgaaatcggtgattacattactacccataagggcattaacttcatcaacttcactggttctactgaagttggtgctagaatttctaagatgacctctatggttcccctgttgatggaattaggtggtaaagatgctgctatcgttttggaagatgctgatttggaattgactgcctctaatatcgttgctggtggttattcttattccggtcaaagatgtactgccgtcaagagaattttggttgttgataaggttgccgacaagctgttggaaaagatcaaagaaaagatgaagaaactgaccgtcggtaacccattggaaaaagatgttgatatcgtccccttgatttcttctaaggctgctgatttcgttatcgaattgattgaagatgccaagtccaaaggtgcagatttgatagttggtggtaatagagaaggcaacttgatctatccaaccttgtttgataacgttaccaccgatatgagattggcttgggaagaaccatttggtccagttttgccaattatcagagttaaggataaggacgaagccattgaaatcgctaacaaatccgaatatggtctgcaatctgctgttttcaccaagaacattaacgatgctttttacgtcgccgataagttggaagttggtactgttcaaatcaacaacaagactgaaagaggtccagataactttccttttatgggtgtaaaagcttccggtattggtacacaaggtatcaagtactccatcgaatctatgtctagaccaaaggccaccattatcaacttgtccattcataactaa

SEQ ID NO: 80 - protein, 486 aa, Clostridium botulinum
MFNHIKDENNTFKNLINGEWVSSRSFVEIKSPLSNSLLGRVPAMTKEEVDIAVQTAKEAQKKWNKITINERAEILYKASDILLENIDELSELMMMEIAKDRKSCRSEVSRTSDFIKFTADTAKNLSGESIPGDSFPGFKNNKVSIVKREPLGVVLAISPFNYPINLSASKIAPGLMAGNSVVLKPATQGSLCGLYLARIFEKAGVPAGVLNTITGKGSEIGDYITTHKGINFINFTGSTEVGARISKMTSMVPLLMELGGKDAAIVLEDADLELTASNIVAGGYSYSGQRCTAVKRILVVDKVADKLLEKIKEKMKKLTVGNPLEKDVDIVPLISSKAADFVIELIEDAKSKGADLIVGGNREGNLIYPTLFDNVTTDMRLAWEEPFGPVLPIIRVKDKDEAIEIANKSEYGLQSAVFTKNINDAFYVADKLEVGTVQINNKTERGPDNFPFMGVKASGIGTQGIKYSIESMSRPKATIINLSIHN

SEQ ID NO: 81 - DNA, 1440 bp, Bacillus cereus
  source 1..1440: mol_type = other DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccgacatcatcatgaaggaagttggtaaaggttacaaggacgccaagaaagaagttgttagaaccgctgatttcatcaggtacactattgaagaggctttacacatgcatggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgcaagaacaagttgccaaattgtctgttggttctcctgaacaagattctactatcgttccattgatcgatgataagtctgccgattttgttcaaggcttggttgatgatgctgttgaaaaaggtgctaccatcgttattggtaacaagagggaaagaaacttgatctacccaaccttgattgatcacgttaccgaagatatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctattgccaacaagattgaaactggttccgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 82 - protein, 479 aa, Bacillus cereus
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLQEQVAKLSVGSPEQDSTIVPLIDDKSADFVQGLVDDAVEKGATIVIGNKRERNLIYPTLIDHVTEDMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 83 - DNA, 1440 bp, Bacillus anthracis
  source 1..1440: mol_type = other DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccgacatcatcatgaaggaagttggtaaaggttacaaggacgccaagaaagaagttgttagaaccgctgatttcatcaggtacactattgaagaggctttacacatgcatggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgaaagaacaagttgccaagttgtctgttggttctcctgaacaagattctactatcgttccattgatcgatgataagtctgccgattttgttcaaggcttggttgatgatgctgttgaaaaaggtgctaccatcgttattggtaacaacagggaaagaaacttgatctacccaaccttgattgatcacgttaccgaagaaatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctattgccaacaagattgaaactggttccgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 84 - protein, 479 aa, Bacillus anthracis
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLKEQVAKLSVGSPEQDSTIVPLIDDKSADFVQGLVDDAVEKGATIVIGNNRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 85 - DNA, 1440 bp, Bacillus thuringiensis
  source 1..1440: mol_type = other DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccaacatcatcatgaaggaagttggtaagggttacaaggatgccaagaaagaagttgttagaaccgccgatttcatcagatacactattgaagaggctttacacatgcacggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgaaagaacaagttgccaagttgtctgttggttctcctgaacaagattctactatcgttccattgatcgatgataagtccgctgattttgttcaaggcttggttgatgatgctgttgaaaaaggtgctaccatcgttattggtaacaagagggaaagaaacttgatctacccaaccttgattgatcacgttaccgaagaaatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctatcgccaacaagattgaaactggttctgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 86 - protein, 479 aa, Bacillus thuringiensis
MTTSNTYKFYLNGEWRESSSGETIEIPSPYLHEVIGQVQAITRGEVDEAIASAKEAQKSWAEASLQDRAKYLYKWADELVNMQDEIANIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLKEQVAKLSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 87 - DNA, 1005 bp, Pyrococcus furiosus
  source 1..1005: mol_type = other DNA
atgaagatcaaggttggtatcaacggttacggtactattggtaaaagagttgcttacgctgttaccaagcaagatgatatggaattgatcggtgttactaagaccaagccagattttgaagcttacagagctaaagaattgggtattccagtttacgctgcttctgaagaatttttgccaagatttgaaaaggccggtttcgaagttgaaggtactttgaatgatttgttggagaaggttgatatcatcgttgatgctactccaggtggtatgggtgaaaaaaacaagcagttgtacgaaaaggctggtgttaaggctatttttcaaggtggtgaaaaagctgaagttgcccaagtttcttttgttgctcaagctaattatgaagccgccttgggtaaagattacgttagagttgtttcttgtaacaccaccggtttggttagaacattgaacgctattaaggattacgtcgattacgtttacgccgttatgattagaagggctgctgatccaaatgatattaagagaggtcctattaacgccatcaagccatctgttactattccatctcatcatggtccagatgttcaaaccgttattccaatcaacattgaaacctccgctttcgttgttccaactaccattatgcatgttcactccatcatggtggaattgaaaaagccattgaccagagaagatgttatcgacatcttcgaaaacaccaccagagttttgttgttcgaaaaagaaaagggtttcgaatccaccgctcaattgattgaatttgctagagacttgcaccgtgaatggaacaacttatacgaaattgccgtctggaaagagtccattaacgtaaagggtaaccgtttgttctacatccaagctgttcatcaagaatccgatgttatcccagaaaacattgatgctattagggccatgttcgaaattgctgaaaaatgggagtctatcaaaaagaccaacaagtccttgggtatcctgaagtaa

SEQ ID NO: 88 - protein, 334 aa, Pyrococcus furiosus
MKIKVGINGYGTIGKRVAYAVTKQDDMELIGVTKTKPDFEAYRAKELGIPVYAASEEFLPRFEKAGFEVEGTLNDLLEKVDIIVDATPGGMGEKNKQLYEKAGVKAIFQGGEKAEVAQVSFVAQANYEAALGKDYVRVVSCNTTGLVRTLNAIKDYVDYVYAVMIRRAADPNDIKRGPINAIKPSVTIPSHHGPDVQTVIPINIETSAFVVPTTIMHVHSIMVELKKPLTREDVIDIFENTTRVLLFEKEKGFESTAQLIEFARDLHREWNNLYEIAVWKESINVKGNRLFYIQAVHQESDVIPENIDAIRAMFEIAEKWESIKKTNKSLGILK